SEQUENCE LISTING (188 sequences)

SEQ ID NO: 1 - DNA, 663 bp, Homo sapiens
atgtccatgggcctggagatcacgggcaccgcgctggccgtgctgggctggctgggcaccatcgtgtgctgcgcgttgcccatgtggcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccgcgccctcatcgtggtggccatcctgctggccgccttcgggctgctagtggcgctggtgggcgcccagtgcaccaactgcgtgcaggacgacacggccaaggccaagatcaccatcgtggcaggcgtgctgttccttctcgccgccctgctcaccctcgtgccggtgtcctggtcggccaacaccattatccgggacttctacaaccccgtggtgcccgaggcgcagaagcgcgagatgggcgcgggcctgtacgtgggctgggcggccgcggcgctgcagctgctggggggcgcgctgctctgctgctcgtgtcccccacgcgagaagaagtacacggccaccaaggtcgtctactccgcgccgcgctccaccggcccgggagccagcctgggcacaggctacgaccgcaaggactacgtctaa

SEQ ID NO: 2 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 3 - DNA, 660 bp, Mus musculus
atgtccatgggcctggagatcaccggcacgtcgctggccgtgctgggctggctgtgcaccatcgtgtgctgcgcccttcccatgtggcgcgtttcggccttcatcggcagcagcatcatcacggcgcagatcacctgggagggcctgtggatgaactgcgtggtgcagagcaccggtcagatgcagtgcaaaatgtacgactcgctgctggccctgccgcaggacctgcaggccgcccgagccctcatcgtggtgtccatcctgctggccgccttcgggctcctcgtggcgctcgtgggcgcccagtgtaccaactgcgtacaagacgagacggccaaggccaagatcaccatcgtggcgggagtgcttttcctgttggcggctctgctcaccttagtaccggtgtcctggtcggccaacaccatcatcagggatttctataacccgttggtgcccgaggcccagaagcgggagatgggagctgggttgtacgtgggctgggctgccgccgcgctgcagttgctagggggcgccttgctgtgttgctcctgcccaccgcgcgacaagtatgcacccaccaagatcctctattctgcgccgcgatccaccggccctggcaccggtaccggcaccgcctacgaccgcaaggactacgtctga

SEQ ID NO: 4 - protein, 219 aa, Mus musculus
MSMGLEITGTSLAVLGWLCTIVCCALPMWRVSAFIGSSIITAQITWEGLWMNCVVQSTGQMQCKMYDSLLALPQDLQAARALIVVSILLAAFGLLVALVGAQCTNCVQDETAKAKITIVAGVLFLLAALLTLVPVSWSANTIIRDFYNPLVPEAQKREMGAGLYVGWAAAALQLLGGALLCCSCPPRDKYAPTKILYSAPRSTGPGTGTGTAYDRKDYV

SEQ ID NO: 5 - DNA, 681 bp, Homo sapiens
atggccaacgcggggctgcagctgttgggcttcattctcgccttcctgggatggatcggcgccatcgtcagcactgccctgccccagtggaggatttactcctatgccggcgacaacatcgtgaccgcccaggccatgtacgaggggctgtggatgtcctgcgtgtcgcagagcaccgggcagatccagtgcaaagtctttgactccttgctgaatctgagcagcacattgcaagcaacccgtgccttgatggtggttggcatcctcctgggagtgatagcaatctttgtggccaccgttggcatgaagtgtatgaagtgcttggaagacgatgaggtgcagaagatgaggatggctgtcattgggggcgcgatatttcttcttgcaggtctggctattttagttgccacagcatggtatggcaatagaatcgttcaagaattctatgaccctatgaccccagtcaatgccaggtacgaatttggtcaggctctcttcactggctgggctgctgcttctctctgccttctgggaggtgccctactttgctgttcctgtccccgaaaaacaacctcttacccaacaccaaggccctatccaaaacctgcaccttccagcgggaaagactacgtggctagcgatatcgcggccgctgactacaaagacgatgacgacaagtga

SEQ ID NO: 6 - protein, 226 aa, Homo sapiens
MANAGLQLLGFILAFLGWIGAIVSTALPQWRIYSYAGDNIVTAQAMYEGLWMSCVSQSTGQIQCKVFDSLLNLSSTLQATRALMVVGILLGVIAIFVATVGMKCMKCLEDDEVQKMRMAVIGGAIFLLAGLAILVATAWYGNRIVQEFYDPMTPVNARYEFGQALFTGWAAASLCLLGGALLCCSCPRKTTSYPTPRPYPKPAPSSGKDYVASDIAAADYKDDDDK

SEQ ID NO: 7 - DNA, 675 bp, Homo sapiens
atggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgactcgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcgccaaggccaagaccatgatcgtggcgggcgtggtgttcctgttggccggccttatggtgatagtgccggtgtcctggacggcccacaacatcatccaagacttctacaatccgctggtggcctccgggcagaagcgggagatgggtgcctcgctctacgtcggctgggccgcctccggcctgctgctccttggcggggggctgctttgctgcaactgtccaccccgcacagacaagccttactccgccaagtattctgctgcccgctctgctgctgccagcaactacgtggctagcgatatcgcggccgctgactacaaagacgatgacgacaagtga

SEQ ID NO: 8 - protein, 224 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYVASDIAAADYKDDDDK

SEQ ID NO: 9 - DNA, 708 bp, Homo sapiens
atggcctctgccggaatgcagatcctgggagtcgtcctgacactgctgggctgggtgaatggcctggtctcctgtgccctgcccatgtggaaggtgaccgctttcatcggcaacagcatcgtggtggcccaggtggtgtgggagggcctgtggatgtcctgcgtggtgcagagcaccggccagatgcagtgcaaggtgtacgactcactgctggcgctgccacaggacctgcaggctgcacgtgccctctgtgtcatcgccctccttgtggccctgttcggcttgctggtctaccttgctggggccaagtgtaccacctgtgtggaggagaaggattccaaggcccgcctggtgctcacctctgggattgtctttgtcatctcaggggtcctgacgctaatccccgtgtgctggacggcgcatgccgtcatccgggacttctataaccccctggtggctgaggcccaaaagcgggagctgggggcctccctctacttgggctgggcggcctcaggccttttgttgctgggtggggggttgctgtgctgcacttgcccctcgggggggtcccagggccccagccattacatggcccgctactcaacatctgcccctgccatctctcgggggccctctgagtaccctaccaagaattacgtcgctagcgatatcgcggccgctgactacaaagacgatgacgacaagtga

SEQ ID NO: 10 - protein, 235 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAVIRDFYNPLVAEAQKRELGASLYLGWAASGLLLLGGGLLCCTCPSGGSQGPSHYMARYSTSAPAISRGPSEYPTKNYVASDIAAADYKDDDDK

SEQ ID NO: 11 - DNA, 15 bp, Mus musculus
ggctacaccatgaac

SEQ ID NO: 12 - protein, 5 aa, Mus musculus
GYTMN

SEQ ID NO: 13 - DNA, 51 bp, Mus musculus
cttattaatccttacaatggtggtactagctacaaccagaagttcaaggac

SEQ ID NO: 14 - protein, 17 aa, Mus musculus
LINPYNGGTSYNQKFKD

SEQ ID NO: 15 - DNA, 30 bp, Mus musculus
gggtcctacggtagtagctactttgactac

SEQ ID NO: 16 - protein, 10 aa, Mus musculus
GSYGSSYFDY

SEQ ID NO: 17 - DNA, 330 bp, Mus musculus
gagctggtgaagcctggagcttcaatgaagatatcctgcaaggcttctggttactcattcactggctacaccatgaactggatgaagcagggccatggaaagaaccttgagtggattggacttattaatccttacaatggtggtactagctacaaccagaagttcaaggacaaggccacattaactttagacaagtcatccagttcagcctacatggagctcctcagtctgacatctgaggactctgcagtctattactgtgcaagagggtcctacggtagtagctactttgactactggggccaaggcaccactctcacagtctcctca

SEQ ID NO: 18 - protein, 110 aa, Mus musculus
ELVKPGASMKISCKASGYSFTGYTMNWMKQGHGKNLEWIGLINPYNGGTSYNQKFKDKATLTLDKSSSSAYMELLSLTSEDSAVYYCARGSYGSSYFDYWGQGTTLTVSS

SEQ ID NO: 19 - DNA, 1389 bp, Mus musculus
  sig_peptide (1)..(84)
atgggatggagctggatctttctcttcctcctgtcaggaactgcaggtgtccactctgaggtccagctgcaacagtctggacctgagctggtgaagcctggagcttcaatgaagatatcctgcaaggcttctggttactcattcactggctacaccatgaactggatgaagcagggccatggaaagaaccttgagtggattggacttattaatccttacaatggtggtactagctacaaccagaagttcaaggacaaggccacattaactttagacaagtcatccagttcagcctacatggagctcctcagtctgacatctgaggactctgcagtctattactgtgcaagagggtcctacggtagtagctactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatgaacacgaatggctcttacttcgtctacagcaagctcaatgtgcagaagagcaactgggaggcaggaaatactttcacctgctctgtcttacatgagggcctgcacaaccaccatactgagaagagcctctcccactctcctggtaaataa

SEQ ID NO: 20 - protein, 462 aa, Mus musculus
  SIGNAL (1)..(28)
MGWSWIFLFLLSGTAGVHSEVQLQQSGPELVKPGASMKISCKASGYSFTGYTMNWMKQGHGKNLEWIGLINPYNGGTSYNQKFKDKATLTLDKSSSSAYMELLSLTSEDSAVYYCARGSYGSSYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 21 - DNA, 993 bp, Homo sapiens
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 330 aa, Homo sapiens [sequence omitted]

SEQ ID NO: 23 - DNA, 51 bp, Mus musculus
aagtccagtcagagccttttatacggtagcaatcaaaagaactacttggcc

SEQ ID NO: 24 - protein, 17 aa, Mus musculus
KSSQSLLYGSNQKNYLA

SEQ ID NO: 25 - DNA, 21 bp, Mus musculus
tgggcatccactagggaatct

SEQ ID NO: 26 - protein, 7 aa, Mus musculus
WASTRES

SEQ ID NO: 27 - DNA, 27 bp, Mus musculus
caacaatattataactttccgtacacg

SEQ ID NO: 28 - protein, 9 aa, Mus musculus
QQYYNFPYT

SEQ ID NO: 29 - DNA, 342 bp, Mus musculus
gacattgtgatgtcacagtctccatcctccctagctgtgtcagttggagagaaggttactatgagttgtaagtccagtcagagccttttatacggtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcaacaatattataactttccgtacacgttcggaggggggaccaagctggaaataaaacgg

SEQ ID NO: 30 - protein, 114 aa, Mus musculus
DIVMSQSPSSLAVSVGEKVTMSCKSSQSLLYGSNQKNYLAWYQQKPGQSPKLLIYWASTRESGVPDRFTGSGSGTDFTLTISSVKAEDLAVYYCQQYYNFPYTFGGGTKLEIKR

SEQ ID NO: 31 - DNA, 723 bp, Mus musculus
  sig_peptide (1)..(60)
atggattcacaggcccaggttcttatgttactgctgctatgggtatctggtacctgtggggacattgtgatgtcacagtctccatcctccctagctgtgtcagttggagagaaggttactatgagttgtaagtccagtcagagccttttatacggtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcaacaatattataactttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 32 - protein, 240 aa, Mus musculus
  SIGNAL (1)..(20)
MDSQAQVLMLLLLWVSGTCGDIVMSQSPSSLAVSVGEKVTMSCKSSQSLLYGSNQKNYLAWYQQKPGQSPKLLIYWASTRESGVPDRFTGSGSGTDFTLTISSVKAEDLAVYYCQQYYNFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 33 - DNA, 321 bp, Homo sapiens
acggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 34 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 15 bp, Mus musculus
gactactacatgaac

SEQ ID NO: 36 - protein, 5 aa, Mus musculus
DYYMN

SEQ ID NO: 37 - DNA, 51 bp, Mus musculus
cgtgttaatcctagtaatggtggtactagctacaaccagaagttcaagggc

SEQ ID NO: 38 - protein, 17 aa, Mus musculus
RVNPSNGGTSYNQKFKG

SEQ ID NO: 39 - DNA, 33 bp, Mus musculus
ggcctagcctactatagtaactcctttgtttac

SEQ ID NO: 40 - protein, 11 aa, Mus musculus
GLAYYSNSFVY

SEQ ID NO: 41 - DNA, 360 bp, Mus musculus
gaggtccagctgcaacagtctggacctgagctggtgaagcctggggcttcagtgaagatgtcctgtaaggcttctggatacacattcactgactactacatgaactgggtgaagcagagtcatggaaagagccttgagtggattggacgtgttaatcctagtaatggtggtactagctacaaccagaagttcaagggcaaggccacattgacagtagacaaatccctcagcacagcctacatgcagctcaacagcctgacatctgaggactctgcggtctattactgtgcaagaggcctagcctactatagtaactcctttgtttactggggccaagggactctggtcactgtctctgca

SEQ ID NO: 42 - protein, 120 aa, Mus musculus
EVQLQQSGPELVKPGASVKMSCKASGYTFTDYYMNWVKQSHGKSLEWIGRVNPSNGGTSYNQKFKGKATLTVDKSLSTAYMQLNSLTSEDSAVYYCARGLAYYSNSFVYWGQGTLVTVSA

SEQ ID NO: 43 - DNA, 1431 bp, Mus musculus
  sig_peptide (1)..(57)
atgggatggagctgggtctttctcttcctcctgtcaggaactgcaggtgtccactctgaggtccagctgcaacagtctggacctgagctggtgaagcctggggcttcagtgaagatgtcctgtaaggcttctggatacacattcactgactactacatgaactgggtgaagcagagtcatggaaagagccttgagtggattggacgtgttaatcctagtaatggtggtactagctacaaccagaagttcaagggcaaggccacattgacagtagacaaatccctcagcacagcctacatgcagctcaacagcctgacatctgaggactctgcggtctattactgtgcaagaggcctagcctactatagtaactcctttgtttactggggccaagggactctggtcactgtctctgcagccaaaacaacacccccatcagtctatccactggcccctgggtgtggagatacaactggttcctccgtgacctctgggtgcctggtcaagggctacttccctgagtcagtgactgtgacttggaactctggatccctgtccagcagtgtgcacaccctctcccaggctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatga

SEQ ID NO: 44 - protein, 476 aa, Mus musculus
  SIGNAL (1)..(19)
MGWSWVFLFLLSGTAGVHSEVQLQQSGPELVKPGASVKMSCKASGYTFTDYYMNWVKQSHGKSLEWIGRVNPSNGGTSYNQKFKGKATLTVDKSLSTAYMQLNSLTSEDSAVYYCARGLAYYSNSFVYWGQGTLVTVSAAKTTPPSVYPLAPGCGDTTGSSVTSGCLVKGYFPESVTVTWNSGSLSSSVHTLSQALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 45 - DNA, 45 bp, Mus musculus
agagccagtgaaagtgttgatagttatggcaatagttttatgcac

SEQ ID NO: 46 - protein, 15 aa, Mus musculus
RASESVDSYGNSFMH

SEQ ID NO: 47 - DNA, 21 bp, Mus musculus
cgtgcatccaacctagaatct

SEQ ID NO: 48 - protein, 7 aa, Mus musculus
RASNLES

SEQ ID NO: 49 - DNA, 27 bp, Mus musculus
cagcaaaataatgaggatccgtggacg

SEQ ID NO: 50 - protein, 9 aa, Mus musculus
QQNNEDPWT

SEQ ID NO: 51 - DNA, 336 bp, Mus musculus
aaaattgtgctgacccaatctccagcttctttggctgtgtctctaaggcagagggccaccatatcctgcagagccagtgaaagtgttgatagttatggcaatagttttatgcactggtaccagcagaaaccaggacagccacccaaactcctcatctatcgtgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaacgg

SEQ ID NO: 52 - protein, 112 aa, Mus musculus
KIVLTQSPASLAVSLRQRATISCRASESVDSYGNSFMHWYQQKPGQPPKLLIYRASNLESGVPARFSGSGSRTDFTLTIDPVEADDAATYYCQQNNEDPWTFGGGTKLEIKR

SEQ ID NO: 53 - DNA, 717 bp, Mus musculus
  sig_peptide (1)..(60)
atggagacagagacactcctgctatgggtgctactgctctgggttccaggttccacaggtaaaattgtgctgacccaatctccagcttctttggctgtgtctctaaggcagagggccaccatatcctgcagagccagtgaaagtgttgatagttatggcaatagttttatgcactggtaccagcagaaaccaggacagccacccaaactcctcatctatcgtgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 54 - protein, 238 aa, Mus musculus
  SIGNAL (1)..(20)
METETLLLWVLLLWVPGSTGKIVLTQSPASLAVSLRQRATISCRASESVDSYGNSFMHWYQQKPGQPPKLLIYRASNLESGVPARFSGSGSRTDFTLTIDPVEADDAATYYCQQNNEDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 55 - DNA, 15 bp, Mus musculus
ggctactttatgaac

SEQ ID NO: 56 - protein, 5 aa, Mus musculus
GYFMN

SEQ ID NO: 57 - DNA, 51 bp, Mus musculus
cgtattaatccttacaatggtgatactttctacaaccagaagttcaagggc

SEQ ID NO: 58 - protein, 17 aa, Mus musculus
RINPYNGDTFYNQKFKG

SEQ ID NO: 59 - DNA, 24 bp, Mus musculus
agtggtgactggtacttcgatgtc

SEQ ID NO: 60 - protein, 8 aa, Mus musculus
SGDWYFDV

SEQ ID NO: 61 - DNA, 351 bp, Mus musculus
gaggttcagctgcagcagtctggacctgagctggtgaagcctggggcttcagtgaagatatcctgcaaggcttctggttactcatttactggctactttatgaactgggtgaagcagagccatggaaagagccttgagtggattggacgtattaatccttacaatggtgatactttctacaaccagaagttcaagggcaaggccacattaactgtagacaaatcctctagcacagcccacatggagctccggagcctgacatctgaggactctgcagtctattattgtgcaagaagtggtgactggtacttcgatgtctggggcgcagggaccacggtcaccgtctcctca

SEQ ID NO: 62 - protein, 117 aa, Mus musculus
EVQLQQSGPELVKPGASVKISCKASGYSFTGYFMNWVKQSHGKSLEWIGRINPYNGDTFYNQKFKGKATLTVDKSSSTAHMELRSLTSEDSAVYYCARSGDWYFDVWGAGTTVTVSS

SEQ ID NO: 63 - DNA, 1416 bp, Mus musculus
  sig_peptide (1)..(57)
atgggatggagctggatctttctctttctcctgtcagtaactgcaggtgtgttctctgaggttcagctgcagcagtctggacctgagctggtgaagcctggggcttcagtgaagatatcctgcaaggcttctggttactcatttactggctactttatgaactgggtgaagcagagccatggaaagagccttgagtggattggacgtattaatccttacaatggtgatactttctacaaccagaagttcaagggcaaggccacattaactgtagacaaatcctctagcacagcccacatggagctccggagcctgacatctgaggactctgcagtctattattgtgcaagaagtggtgactggtacttcgatgtctggggcgcagggaccacggtcaccgtctcctcagccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatga

SEQ ID NO: 64 - protein, 471 aa, Mus musculus
  SIGNAL (1)..(19)
MGWSWIFLFLLSVTAGVFSEVQLQQSGPELVKPGASVKISCKASGYSFTGYFMNWVKQSHGKSLEWIGRINPYNGDTFYNQKFKGKATLTVDKSSSTAHMELRSLTSEDSAVYYCARSGDWYFDVWGAGTTVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 65 - DNA, 45 bp, Mus musculus
agggccagcaaaagtgtcagtacatctagctatagttacatgcac

SEQ ID NO: 66 - protein, 15 aa, Mus musculus
RASKSVSTSSYSYMH

SEQ ID NO: 67 - DNA, 21 bp, Mus musculus
tttgcatcctacctagaatct

SEQ ID NO: 68 - protein, 7 aa, Mus musculus
FASYLES

SEQ ID NO: 69 - DNA, 27 bp, Mus musculus
caacacagtagggagtttcctcggacg

SEQ ID NO: 70 - protein, 9 aa, Mus musculus
PVEEEFPRT

SEQ ID NO: 71 - DNA, 336 bp, Mus musculus
gacattgtgctgacacagtctcctgcttccttagctgtatctctggggcagagggccaccatctcctgcagggccagcaaaagtgtcagtacatctagctatagttacatgcactggtaccaacagaaaccaggacagccacccaaactcctcatcaagtttgcatcctacctagaatctggggttcctgccaggttcagtggcagtgggtctgggacagacttcaccctcaacatccatcctgtggaggaggaggatgctgcaacatattactgtcaacacagtagggagtttcctcggacgttcggtggaggcaccaagctggaaatcaaacgg

SEQ ID NO: 72 - protein, 112 aa, Mus musculus
DIVLTQSPASLAVSLGQRATISCRASKSVSTSSYSYMHWYQQKPGQPPKLLIKFASYLESGVPARFSGSGSGTDFTLNIHPVEEEDAATYYCQHSREFPRTFGGGTKLEIKR

SEQ ID NO: 73 - DNA, 717 bp, Mus musculus
  sig_peptide (1)..(60)
atggagacagacacactcctgctatgggtgctgctgctctgggttccaggttccactggtgacattgtgctgacacagtctcctgcttccttagctgtatctctggggcagagggccaccatctcctgcagggccagcaaaagtgtcagtacatctagctatagttacatgcactggtaccaacagaaaccaggacagccacccaaactcctcatcaagtttgcatcctacctagaatctggggttcctgccaggttcagtggcagtgggtctgggacagacttcaccctcaacatccatcctgtggaggaggaggatgctgcaacatattactgtcaacacagtagggagtttcctcggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 74 - protein, 238 aa, Mus musculus
  SIGNAL (1)..(20)
METDTLLLWVLLLWVPGSTGDIVLTQSPASLAVSLGQRATISCRASKSVSTSSYSYMHWYQQKPGQPPKLLIKFASYLESGVPARFSGSGSGTDFTLNIHPVEEEDAATYYCQHSREFPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 75 - DNA, 15 bp, Mus musculus
ggctactttatgaac

SEQ ID NO: 76 - protein, 5 aa, Mus musculus
GYFMN

SEQ ID NO: 77 - DNA, 51 bp, Mus musculus; identical to SEQ ID NO: 57

SEQ ID NO: 78 - protein, 17 aa, Mus musculus
RINPYNGDTFYNQKFKG

SEQ ID NO: 79 - DNA, 33 bp, Mus musculus
ggagatggttactacgtgacgagtcttgcttac

SEQ ID NO: 80 - protein, 11 aa, Mus musculus
GDGYYVTSLAY

SEQ ID NO: 81 - DNA, 360 bp, Mus musculus
gaggttcagctgcagcagtctggacctgagctggtgaagcctggggcttcagtgaagatatcctgcaaggcttctggttactcatttactggctactttatgaactgggtgaagcagagccatggaaagagccttgagtggcttggacgtattaatccttacaatggtgatactttctacaaccagaagttcaagggcaaggccacattaactgtagacaaatcctctaacacagcccacatggagctccggagcctgacatctgaggactctgcagtctattattgtgcaagaggagatggttactacgtgacgagtcttgcttactggggccaagggactctggtcactgtctctgca

SEQ ID NO: 82 - protein, 120 aa, Mus musculus
EVQLQQSGPELVKPGASVKISCKASGYSFTGYFMNWVKQSHGKSLEWLGRINPYNGDTFYNQKFKGKATLTVDKSSNTAHMELRSLTSEDSAVYYCARGDGYYVTSLAYWGQGTLVTVSA

SEQ ID NO: 83 - DNA, 1392 bp, Mus musculus
  sig_peptide (1)..(57)
atgggatggagctggatctttctctttctcctgtcagtaactgcaggtgtgttctctgaggttcagctgcagcagtctggacctgagctggtgaagcctggggcttcagtgaagatatcctgcaaggcttctggttactcatttactggctactttatgaactgggtgaagcagagccatggaaagagccttgagtggcttggacgtattaatccttacaatggtgatactttctacaaccagaagttcaagggcaaggccacattaactgtagacaaatcctctaacacagcccacatggagctccggagcctgacatctgaggactctgcagtctattattgtgcaagaggagatggttactacgtgacgagtcttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatgaacacgaatggctcttacttcgtctacagcaagctcaatgtgcagaagagcaactgggaggcaggaaatactttcacctgctctgtcttacatgagggcctgcacaaccaccatactgagaagagcctctcccactctcctggtaaataa

SEQ ID NO: 84 - protein, 463 aa, Mus musculus
  SIGNAL (1)..(19)
MGWSWIFLFLLSVTAGVFSEVQLQQSGPELVKPGASVKISCKASGYSFTGYFMNWVKQSHGKSLEWLGRINPYNGDTFYNQKFKGKATLTVDKSSNTAHMELRSLTSEDSAVYYCARGDGYYVTSLAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 85 - DNA, 33 bp, Mus musculus
aaggccagtgagaatgtggttagttatgtatcc

SEQ ID NO: 86 - protein, 11 aa, Mus musculus
KASENVVSYVS

SEQ ID NO: 87 - DNA, 21 bp, Mus musculus
ggggcatccaaccggtacact

SEQ ID NO: 88 - protein, 7 aa, Mus musculus
GASNRYT

SEQ ID NO: 89 - DNA, 27 bp, Mus musculus
ggacagagttacagctatcctctcacg

SEQ ID NO: 90 - protein, 9 aa, Mus musculus
GQSYSYPLT

SEQ ID NO: 91 - DNA, 324 bp, Mus musculus
aacattgtaatgacccaatctcccaaatccatgtccatgtcagtaggagagagggtcaccttgagctgcaaggccagtgagaatgtggttagttatgtatcctggtttcaacagaaaccagagcagtctcctaaactgctgatatatggggcatccaaccggtacactggggtccccgatcgcttcacaggcagtggatctgcaacagatttcactctgaccatcagcagtgtgcaggctgaagaccttgcagattattactgtggacagagttacagctatcctctcacgttcggtgctgggaccaagctggagctgaaacgg

SEQ ID NO: 92 - protein, 108 aa, Mus musculus
NIVMTQSPKSMSMSVGERVTLSCKASENVVSYVSWFQQKPEQSPKLLIYGASNRYTGVPDRFTGSGSATDFTLTISSVQAEDLADYYCGQSYSYPLTFGAGTKLELKR

SEQ ID NO: 93 - DNA, 717 bp, Mus musculus
  sig_peptide (1)..(72)
atgggcatcaagatggaatcacagactctggtcttcatatccatactgctctggttatatggtgctgatgggaacattgtaatgacccaatctcccaaatccatgtccatgtcagtaggagagagggtcaccttgagctgcaaggccagtgagaatgtggttagttatgtatcctggtttcaacagaaaccagagcagtctcctaaactgctgatatatggggcatccaaccggtacactggggtccccgatcgcttcacaggcagtggatctgcaacagatttcactctgaccatcagcagtgtgcaggctgaagaccttgcagattattactgtggacagagttacagctatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 94 - protein, 238 aa, Mus musculus
  SIGNAL (1)..(24)
MGIKMESQTLVFISILLWLYGADGNIVMTQSPKSMSMSVGERVTLSCKASENVVSYVSWFQQKPEQSPKLLIYGASNRYTGVPDRFTGSGSATDFTLTISSVQAEDLADYYCGQSYSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 95 - DNA, 15 bp, Mus musculus
gactactacatgaac

SEQ ID NO: 96 - protein, 5 aa, Mus musculus
DYYMN

SEQ ID NO: 97 - DNA, 51 bp, Mus musculus
cgtgttaatcctagcaatggtggtactagctacaaccagaagttcaagggc

SEQ ID NO: 98 - protein, 17 aa, Mus musculus
RVNPSNGGTSYNQKFKG

SEQ ID NO: 99 - DNA, 33 bp, Mus musculus
ggcctagcctactatagtaactcctttacttac

SEQ ID NO: 100 - protein, 11 aa, Mus musculus
GLAYYSNSFTY

SEQ ID NO: 101 - DNA, 360 bp, Mus musculus
gaggtccagctgcaacagtctggacctgagctggtgaagcctggggcttcagtgaagatgtcctgtaaggcttctggatacacattcactgactactacatgaactgggtgaagcagagtcatggaaagagccttgagtggattggacgtgttaatcctagcaatggtggtactagctacaaccagaagttcaagggcaaggccacattgacagtagacaaatccctcagcacagcctacatgcagctcaacagcctgacatctgaggactctgcggtctattactgtgcaagaggcctagcctactatagtaactcctttacttactggggccaagggactctggtcactgtctctgca

SEQ ID NO: 102 - protein, 120 aa, Mus musculus
EVQLQQSGPELVKPGASVKMSCKASGYTFTDYYMNWVKQSHGKSLEWIGRVNPSNGGTSYNQKFKGKATLTVDKSLSTAYMQLNSLTSEDSAVYYCARGLAYYSNSFTYWGQGTLVTVSA

SEQ ID NO: 103 - DNA, 1425 bp, Mus musculus
  sig_peptide (1)..(57)
atgggatggagctgggtctttctcttcctcctgtcaggaactgcaggtgtccactctgaggtccagctgcaacagtctggacctgagctggtgaagcctggggcttcagtgaagatgtcctgtaaggcttctggatacacattcactgactactacatgaactgggtgaagcagagtcatggaaagagccttgagtggattggacgtgttaatcctagcaatggtggtactagctacaaccagaagttcaagggcaaggccacattgacagtagacaaatccctcagcacagcctacatgcagctcaacagcctgacatctgaggactctgcggtctattactgtgcaagaggcctagcctactatagtaactcctttacttactggggccaagggactctggtcactgtctctgcagccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatga

SEQ ID NO: 104 - protein, 474 aa, Mus musculus
  SIGNAL (1)..(19)
MGWSWVFLFLLSGTAGVHSEVQLQQSGPELVKPGASVKMSCKASGYTFTDYYMNWVKQSHGKSLEWIGRVNPSNGGTSYNQKFKGKATLTVDKSLSTAYMQLNSLTSEDSAVYYCARGLAYYSNSFTYWGQGTLVTVSAAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 105 - DNA, 45 bp, Mus musculus; identical to SEQ ID NO: 45

SEQ ID NO: 106 - protein, 15 aa, Mus musculus
RASESVDSYGNSFMH

SEQ ID NO: 107 - DNA, 21 bp, Mus musculus
cgtgcatccaacctagaatct

SEQ ID NO: 108 - protein, 7 aa, Mus musculus
RASNLES

SEQ ID NO: 109 - DNA, 27 bp, Mus musculus
cagcaaaataatgaggatccgtggacg

SEQ ID NO: 110 - protein, 9 aa, Mus musculus
QQNNEDPWT

SEQ ID NO: 111 - DNA, 336 bp, Mus musculus; identical to SEQ ID NO: 51

SEQ ID NO: 112 - protein, 112 aa, Mus musculus; identical to SEQ ID NO: 52

SEQ ID NO: 113 - DNA, 717 bp, Mus musculus; identical to SEQ ID NO: 53
  sig_peptide (1)..(60)

SEQ ID NO: 114 - protein, 238 aa, Mus musculus; identical to SEQ ID NO: 54
  SIGNAL (1)..(20)

SEQ ID NO: 115 - DNA, 867 bp, Artificial
  An artificially synthesized nucleotide sequence
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccaggaattcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccggtcgactcaccatcatcatcatcattaa

SEQ ID NO: 116 - protein, 288 aa, Artificial
  An artificially synthesized peptide sequence
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPGIRVSAFIGSNIITSQNIWEGLWMNCVVQSTGQMQCKVYDSLLALPQDLQAARSTHHHHHH

SEQ ID NO: 117 - protein, 261 aa, Artificial
  An artificially synthesized peptide sequence
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPGIPWSANTIIRDFYNPVVPEAQKREMSTHHHHHH

SEQ ID NO: 118 - DNA, 786 bp, Artificial
  An artificially synthesized nucleotide sequence
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccaggaattccctggtcggccaacaccattatccgggacttctacaaccccgtggtgcccgaggcgcagaagcgcgagatgtcgactcaccatcatcatcatcattaa

SEQ ID NO: 119 - DNA, 30 bp, Homo sapiens
cggccaccatgtccatgggcctggagatca

SEQ ID NO: 120 - DNA, 30 bp, Homo sapiens
gtctgtcccttagacgtagtccttgcggtc

SEQ ID NO: 121 - DNA, 34 bp, Mus musculus
gcgaattccaccatgtccatgggcctggagatca

SEQ ID NO: 122 - DNA, 33 bp, Mus musculus
gcgatatctgtcctcttccagcctagcaagcag

SEQ ID NO: 123 - DNA, 30 bp, Homo sapiens
atggccaacgcggggctgcagctgttgggc

SEQ ID NO: 124 - DNA, 30 bp, Homo sapiens
tgtgtcacacgtagtctttcccgctggaag

SEQ ID NO: 125 - DNA, 27 bp, Homo sapiens
gaacaatggcctccatggggctacagg

SEQ ID NO: 126 - DNA, 29 bp, Homo sapiens
aggagggtggactctgttcttgctagcag

SEQ ID NO: 127 - DNA, 27 bp, Homo sapiens
catggcctctgccggaatgcagatcct

SEQ ID NO: 128 - DNA, 27 bp, Homo sapiens
cccaaagctgttgggcactgccacttc

SEQ ID NO: 129 - DNA, 28 bp, Mus musculus
ccatggagttagtttgggcagcagatcc

SEQ ID NO: 130 - DNA, 24 bp, Mus musculus
caggggccagtggatagaccgatg

SEQ ID NO: 131 - DNA, 24 bp, Mus musculus
caggggccagtggatagactgatg

SEQ ID NO: 132 - DNA, 27 bp, Mus musculus
ggcacctccagatgttaactgctcact

SEQ ID NO: 133 - DNA, 27 bp, Mus musculus
tcgagctcttcagaggaaggtggaaac

SEQ ID NO: 134 - protein, 51 aa, Homo sapiens
RVTAFIGSNIVTSQTIWEGLWMNCVVQSTGQMQCKVYDSLLALPQDLQAAR

SEQ ID NO: 135 - protein, 51 aa, Homo sapiens
KVTAFIGNSIVVAQVVWEGLWMSCVVQSTGQMQCKVYDSLLALPQDLQAAR

SEQ ID NO: 136 - protein, 51 aa, Homo sapiens; identical to SEQ ID NO: 135

SEQ ID NO: 137 - protein, 51 aa, Homo sapiens
RVSAFIENNIVVFENFWEGLWMNCVRQANIRMQCKIYDSLLALSPDLQAAR

SEQ ID NO: 138 - protein, 51 aa, Homo sapiens
QVTAFLDHNIVTAQTTWKGLWMSCVVQSTGHMQCKVYDSVLALSTEVQAAR

SEQ ID NO: 139 - protein, 51 aa, Homo sapiens
RVSAFVGSNIIVFERLWEGLWMNCIRQARVRLQCKFYSSLLALPPALETAR

SEQ ID NO: 140 - protein, 51 aa, Homo sapiens
RIYSYAGDNIVTAQAMYEGLWMSCVSQSTGQIQCKVFDSLLNLSSTLQATR

SEQ ID NO: 141 - protein, 51 aa, Homo sapiens
QMSSYAGDNIITAQAMYKGLWMDCVTQSTGMMSCKMYDSVLALSAALQATR

SEQ ID NO: 142 - protein, 51 aa, Homo sapiens
RRTAHVGTNILTAVSYLKGLWMECVWHSTGIYQCQIYRSLLALPQDLQAAR

SEQ ID NO: 143 - protein, 49 aa, Homo sapiens
SSYAGDAIITAVGLYEGLWMSCASQSTGQVQCKLYDSLLALDGHIQSAR

SEQ ID NO: 144 - protein, 51 aa, Homo sapiens
KTSSYVGASIVTAVGFSKGLWMECATHSTGITQCDIYSTLLGLPADIQAAQ

SEQ ID NO: 145 - protein, 51 aa, Homo sapiens
KVNVDVDSNIITAIVQLHGLWMDCTWYSTGMFSCALKHSILSLPIHVQAAR

SEQ ID NO: 146 - protein, 22 aa, Homo sapiens
VANAIIRDFYNSIVNVAQKREL

SEQ ID NO: 147 - protein, 22 aa, Homo sapiens
TAHAIIQDFYNPLVAEALKREL

SEQ ID NO: 148 - protein, 22 aa, Homo sapiens
TANIIIRDFYNPAIHIGQKREL

SEQ ID NO: 149 - protein, 4 aa, Artificial
  An artificially synthesized peptide sequence
GGGS

SEQ ID NO: 150 - protein, 4 aa, Artificial
  An artificially synthesized peptide sequence
SGGG

SEQ ID NO: 151 - protein, 5 aa, Artificial
  An artificially synthesized peptide sequence
GGGGS

SEQ ID NO: 152 - protein, 5 aa, Artificial
  An artificially synthesized peptide sequence
SGGGG

SEQ ID NO: 153 - protein, 6 aa, Artificial
  An artificially synthesized peptide sequence
GGGGGS

SEQ ID NO: 154 - protein, 6 aa, Artificial
  An artificially synthesized peptide sequence
SGGGGG

SEQ ID NO: 155 - protein, 7 aa, Artificial
  An artificially synthesized peptide sequence
GGGGGGS

SEQ ID NO: 156 - protein, 7 aa, Artificial
  An artificially synthesized peptide sequence
SGGGGGG

SEQ ID NO: 157 - protein, 5 aa, Artificial
  An artificially synthesized peptide sequence
GGGGS

SEQ ID NO: 158 - protein, 5 aa, Artificial
  An artificially synthesized peptide sequence
SGGGG

SEQ ID NO: 159 - DNA, 3876 bp, Artificial
  An artificially synthesized nucleotide sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgtgagtcattagggactttccaatgggttttgcccagtacataaggtcaatgggaggtaagccaatgggtttttcccattactggcacctagctgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcaaagcttgcccgggcgaattcgattcgggccaccatgtccatgggcctggagatcacgggcaccgcgctggccgtgctgggctggctgggcaccatcgtgtgctgcgcgttgcccatgtggcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccgcgccctcatcgtggtggccatcctgctggccgccttcgggctgctagtggcgctggtgggcgcccagtgcaccaactgcgtgcaggacgacacggccaaggccaagatcaccatcgtggcaggcgtgctgttccttctcgccgccctgctcaccctcgtgccggtgtcctggtcggccaacaccattatccgggacttctacaaccccgtggtgcccgaggcgcagaagcgcgagatgggcgcgggcctgtacgtgggctgggcggccgcggcgctgcagctgctggggggcgcgctgctctgctgctcgtgtcccccacgcgagaagaagtacacggccaccaaggtcgtctactccgcgccgcgctccaccggcccgggagccagcctgggcacaggctacgaccgcaaggactacgtctaagggtcagacaatcactagtgaattcgtcgacgctagcgatatcgcggccgctctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 160 - DNA, 714 bp, Artificial
  An artificially synthesized nucleotide sequence
atggccaacgcggggctgcagctgttgggcttcattctcgccttcctgggatggatcggcgccatcgtcagcactgccctgccccagtggaggatttactcctatgccggcgacaacatcgtgaccgcccaggccatgtacgaggggctgtggatgtcctgcgtgtcgcagagcaccgggcagatccagtgcaaagtctttgactccttgctgaatctgagcagcacattgcaagcaacccgtgccttgatggtggttggcatcctcctgggagtgatagcaatctttgtggccaccgttggcatgaagtgtatgaagtgcttggaagacgatgaggtgcagaagatgaggatggctgtcattgggggcgcgatatttcttctcgccgccctgctcaccctcgtgccggtgtcctggtcggccaacaccattatccgggacttctacaaccccgtggtgcccgaggcgcagaagcgcgagatgggcgcgggcctgtacgtgggctgggcggccgcggcgctgcagctgctggggggcgcgctgctctgctgctcgtgtcccccacgcgagaagaagtacacggccaccaaggtcgtctactccgcgccgcgctccaccggcccgggagccagcctgggcacaggctacgaccgcaaggactacgtcgctagcgatatcgcggccgctgactacaaagacgatgacgacaagtga

SEQ ID NO: 161 - protein, 237 aa, Artificial
  Synthetic Construct
MANAGLQLLGFILAFLGWIGAIVSTALPQWRIYSYAGDNIVTAQAMYEGLWMSCVSQSTGQIQCKVFDSLLNLSSTLQATRALMVVGILLGVIAIFVATVGMKCMKCLEDDEVQKMRMAVIGGAIFLLAALLTLVPVSWSANTIIRDFYNPVVPEAQKREMGAGLYVGWAAAALQLLGGALLCCSCPPREKKYTATKVVYSAPRSTGPGASLGTGYDRKDYVASDIAAADYKDDDDK

SEQ ID NO: 162 - DNA, 675 bp, Artificial
  An artificially synthesized nucleotide sequence
atgtccatgggcctggagatcacgggcaccgcgctggccgtgctgggctggctgggcaccatcgtgtgctgcgcgttgcccatgtggcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccgcgccctcatcgtggtggccatcctgctggccgccttcgggctgctagtggcgctggtgggcgcccagtgcaccaactgcgtgcaggacgacacggccaaggccaagatcaccatcgtggcaggcgtgctgttccttcttgcaggtctggctattttagttgccacagcatggtatggcaatagaatcgttcaagaattctatgaccctatgaccccagtcaatgccaggtacgaatttggtcaggctctcttcactggctgggctgctgcttctctctgccttctgggaggtgccctactttgctgttcctgtccccgaaaaacaacctcttacccaacaccaaggccctatccaaaacctgcaccttccagcgggaaagactacgtggctagcgatatcgcggccgctgactacaaagacgatgacgacaagtga

SEQ ID NO: 163 - protein, 224 aa, Artificial
  Synthetic Construct
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAGLAILVATAWYGNRIVQEFYDPMTPVNARYEFGQALFTGWAAASLCLLGGALLCCSCPRKTTSYPTPRPYPKPAPSSGKDYVASDIAAADYKDDDDK

SEQ ID NO: 164 - DNA, 351 bp, Mus musculus
  CDS (1)..(351)
gaggtccagctgcagcagtctggacctgagctggtgaagcctggggcttcagtgaagatgtcctgcaaggcttctggttactcctttactggctactttatgaactgggtgaagcagagccatggaaagagccttgagtggattggacgtattaatccttacaatggtgatactttctacaaccagaagttcaagggcaaggccacattgactgtagacaaatcctctagcacagcccacatggagctccggagcctgacatctgaggactctgcactctattattgtgcaagatctggtaactatgttatggactactggggtcaaggaacctcagtcaccgtctcctca

SEQ ID NO: 165 - protein, 117 aa, Mus musculus
EVQLQQSGPELVKPGASVKMSCKASGYSFTGYFMNWVKQSHGKSLEWIGRINPYNGDTFYNQKFKGKATLTVDKSSSTAHMELRSLTSEDSALYYCARSGNYVMDYWGQGTSVTVSS

SEQ ID NO: 166 - DNA, 15 bp, Mus musculus
  CDS (1)..(15)
ggctactttatgaac

SEQ ID NO: 167 - protein, 5 aa, Mus musculus
GYFMN

SEQ ID NO: 168 - DNA, 51 bp, Mus musculus; identical to SEQ ID NO: 57
  CDS (1)..(51)

SEQ ID NO: 169 - protein, 17 aa, Mus musculus
RINPYNGDTFYNQKFKG

SEQ ID NO: 170 - DNA, 24 bp, Mus musculus
  CDS (1)..(24)
tctggtaactatgttatggactac

SEQ ID NO: 171 - protein, 8 aa, Mus musculus
SGNYVMDY

SEQ ID NO: 172 - DNA, 1401 bp, Mus musculus
  CDS (1)..(1398)
  sig_peptide (1)..(57)
  mat_peptide (58)..(1398)
atgggatggagctggatctttctcttccttatgtcaggaacggcaggtgtcctctctgaggtccagctgcagcagtctggacctgagctggtgaagcctggggcttcagtgaagatgtcctgcaaggcttctggttactcctttactggctactttatgaactgggtgaagcagagccatggaaagagccttgagtggattggacgtattaatccttacaatggtgatactttctacaaccagaagttcaagggcaaggccacattgactgtagacaaatcctctagcacagcccacatggagctccggagcctgacatctgaggactctgcactctattattgtgcaagatctggtaactatgttatggactactggggtcaaggaacctcagtcaccgtctcctcagctacaacaacagccccatctgtctatcccttggtccctggctgcagtgacacatctggatcctcggtgacactgggatgccttgtcaaaggctacttccctgagccggtaactgtaaaatggaactatggagccctgtccagcggtgtgcgcacagtctcatctgtcctgcagtctgggttctattccctcagcagcttggtgactgtaccctccagcacctggcccagccagactgtcatctgcaacgtagcccacccagccagcaagactgagttgatcaagagaatcgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaatga

SEQ ID NO: 173 - protein, 466 aa, Mus musculus
MGWSWIFLFLMSGTAGVLSEVQLQQSGPELVKPGASVKMSCKASGYSFTGYFMNWVKQSHGKSLEWIGRINPYNGDTFYNQKFKGKATLTVDKSSSTAHMELRSLTSEDSALYYCARSGNYVMDYWGQGTSVTVSSATTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 174 - DNA, 1401 bp, Artificial
  An artificially synthesized nucleotide sequence
atgggatggagctggatctttctcttccttatgtcaggaacggcaggtgtcctctctgaggtccagctgcagcagtctggacctgagctggtgaagcctggggcttcagtgaagatgtcctgcaaggcttctggttactcctttactggctactttatgaactgggtgaagcagagccatggaaagagccttgagtggattggacgtattaatccttacaatggtgatactttctacaaccagaagttcaagggcaaggccacattgactgtagacaaatcctctagcacagcccacatggagctccggagcctgacatctgaggactctgcactctattattgtgcaagatctggtaactatgttatggactactggggtcaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 175 - protein, 466 aa, Artificial
  Synthetic Construct
MGWSWIFLFLMSGTAGVLSEVQLQQSGPELVKPGASVKMSCKASGYSFTGYFMNWVKQSHGKSLEWIGRINPYNGDTFYNQKFKGKATLTVDKSSSTAHMELRSLTSEDSALYYCARSGNYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - DNA, 336 bp, Mus musculus
  CDS (1)..(336)
gacattgtgctcacccaatctccagcttctttggctgtgtctctagggcagagtgtcaccatctcctgcagagccagtgaaagtgttgaatattatggcactagtttaatgcagtggtaccaacagaaaccaggacagccacccaaactcctcatctatggtgcatccaacgtagaatctggggtccctgccaggtttagtggcagtgggtctgggacagacttcagcctcaacatccatcctgtggaggaggatgatattgcaatgtatttctgtcagcaaagtaggaaggttccgtggacgttcggtggaggcaccaagctggaaatcaaacgg

SEQ ID NO: 177 - protein, 112 aa, Mus musculus
DIVLTQSPASLAVSLGQSVTISCRASESVEYYGTSLMQWYQQKPGQPPKLLIYGASNVESGVPARFSGSGSGTDFSLNIHPVEEDDIAMYFCQQSRKVPWTFGGGTKLEIKR

SEQ ID NO: 178 - DNA, 45 bp, Mus musculus
  CDS (1)..(45)
agagccagtgaaagtgttgaatattatggcactagtttaatgcag

SEQ ID NO: 179 - protein, 15 aa, Mus musculus
RASESVEYYGTSLMQ

SEQ ID NO: 180 - DNA, 21 bp, Mus musculus
  CDS (1)..(21)
ggtgcatccaacgtagaatct

SEQ ID NO: 181 - protein, 7 aa, Mus musculus
GASNVES

SEQ ID NO: 182 - DNA, 27 bp, Mus musculus
  CDS (1)..(27)
cagcaaagtaggaaggttccgtggacg

SEQ ID NO: 183 - protein, 9 aa, Mus musculus
QQSRKVPWT

SEQ ID NO: 184 - DNA, 717 bp, Mus musculus
  CDS (1)..(714)
  sig_peptide (1)..(60)
  mat_peptide (61)..(714)
atggagacagacacactcctgctatgggtgctgctgctctgggttccaggctccactggtgacattgtgctcacccaatctccagcttctttggctgtgtctctagggcagagtgtcaccatctcctgcagagccagtgaaagtgttgaatattatggcactagtttaatgcagtggtaccaacagaaaccaggacagccacccaaactcctcatctatggtgcatccaacgtagaatctggggtccctgccaggtttagtggcagtgggtctgggacagacttcagcctcaacatccatcctgtggaggaggatgatattgcaatgtatttctgtcagcaaagtaggaaggttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 185 - protein, 238 aa, Mus musculus
METDTLLLWVLLLWVPGSTGDIVLTQSPASLAVSLGQSVTISCRASESVEYYGTSLMQWYQQKPGQPPKLLIYGASNVESGVPARFSGSGSGTDFSLNIHPVEEDDIAMYFCQQSRKVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 186 - DNA, 717 bp, Artificial
  An artificially synthesized nucleotide sequence
atggagacagacacactcctgctatgggtgctgctgctctgggttccaggctccactggtgacattgtgctcacccaatctccagcttctttggctgtgtctctagggcagagtgtcaccatctcctgcagagccagtgaaagtgttgaatattatggcactagtttaatgcagtggtaccaacagaaaccaggacagccacccaaactcctcatctatggtgcatccaacgtagaatctggggtccctgccaggtttagtggcagtgggtctgggacagacttcagcctcaacatccatcctgtggaggaggatgatattgcaatgtatttctgtcagcaaagtaggaaggttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 187 - protein, 238 aa, Artificial
  Synthetic Construct
METDTLLLWVLLLWVPGSTGDIVLTQSPASLAVSLGQSVTISCRASESVEYYGTSLMQWYQQKPGQPPKLLIYGASNVESGVPARFSGSGSGTDFSLNIHPVEEDDIAMYFCQQSRKVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - DNA, 25 bp, Artificial
  An artificially synthesized primer sequence
atgtgtcactgcagccagggaccaa

(19) United States
(12) Patent Application Publication
(10) Pub. No.: US 2010/0111852 A1
(43) Pub. Date: May 6, 2010

(54) Anti-Claudin 3 Monoclonal Antibody and Treatment and Diagnosis of Cancer Using the Same

(75) Inventor: Kenji YOSHIDA, Toyko (JP)

Correspondence Address: STERNE, KESSLER, GOLDSTEIN & FOX P.L.L.C., 1100 NEW YORK AVENUE, N.W., WASHINGTON, DC, 20005, US

(73) Assignee: Forerunner Pharma Research Co., Ltd., Tokyo (JP)

(21) Appl. No.: 12/519,337

(22) PCT Filed: Dec. 14, 2007
(86) PCT No.: PCT/JP2007/074081

§ 371 Date: Sep. 18, 2009

(30) Foreign Application Priority Data
Dec. 14, 2006 (JP) 2006-336555

(51) Int. Cl. A61K 51/00; C07K 16/18; A61P 35/04; G01N 33/574; A61K 39/395

(52) U.S. Cl. 424/1.49; 530/387.9; 530/391.7; 530/387.3; 435/7.23; 424/139.1

(57) ABSTRACT

Monoclonal antibodies that bind specifically to Claudin 3 expressed on cell surface are provided. The antibodies of the present invention are useful for diagnosis of cancers that have enhanced expression of Claudin 3, such as ovarian cancer, prostate cancer, breast cancer, uterine cancer, liver cancer, lung cancer, pancreatic cancer, stomach cancer, bladder cancer, and colon cancer. The present invention provides monoclonal antibodies showing cytotoxic effects against cells of these cancers. Methods for inducing cell injury in Claudin 3-expressing cells and methods for suppressing proliferation of Claudin 3-expressing cells by contacting Claudin 3-expressing cells with a Claudin 3-binding antibody are disclosed. The present application also discloses methods for diagnosis or treatment of cancers.

### TECHNICAL FIELD

The present invention relates to methods for diagnosis and treatment of cancer, as well as cell proliferation-suppressing and anticancer agents.

### BACKGROUND ART

In recent years, cancer treatment that uses monoclonal antibodies as therapeutic agents, by utilizing the characteristics of monoclonal antibodies, i.e., high target specificity and low incidence of side-effects, is receiving attention. The main antitumor mechanisms of antibodies used as therapeutic agents include, for example, the following:

discrimination of tumor cells from normal cells by antibodies;

binding of effector cells having cytotoxic activity to antibodies specifically bound to antigens expressed on tumor cells, or formation of complement complexes that bind to the antibodies; and

effector cell- or complement-mediated cytotoxic activity against tumor cells.

Examples of antibodies used for cancer treatment include, trastuzumab which is an antibody for breast cancer treatment targeting HER-2, and rituximab which is an antibody for non-Hodgkin lymphoma treatment targeting CD20. However, the number of antibodies actually showing clinical efficacy is very few at present. Therefore, the types of cancers that could be applied to antibody therapy are very limited at this time. It is highly desirable to develop antibody therapeutic agents with few side effects and high anti-tumor efficiency, and to establish new therapeutic methods against cancers for which there are few therapeutic options and the currently available therapeutic agents are ineffective.

Claudin 3 is a protein that belongs to the Claudin family. It is localized at tight junctions, and has a characteristic role in eliminating the intercellular space at tight junctions. “Tight junction” refers to a rigid structure that links adjacent cell membranes in tissues of organisms such as animals. Claudin 3 is a structural protein that regulates the intercellular permeability of small solutes such as ions. It has been reported that the expression of the Claudin 3 molecule is elevated in many cancer tissues such as ovarian cancer, prostate cancer, breast cancer, uterine cancer, liver cancer, lung cancer, pancreatic cancer, stomach cancer, bladder cancer, and colon cancer tissues (Non-patent Documents 1-6). The Swedish Human Protein Atlas (HPA) Web site (http://www.proteinatlas.org/) is available as a reference for the Claudin 3 expression profile. It has been shown that the expression of Claudin 3 and Claudin 4 is particularly elevated in chemotherapy-resistant and/or recurrent uterine cancer, which is considered to have the highest fatality among gynecological cancers in the United States. As with Claudin 3, Claudin 4 is a protein of the Claudin family. To date, the 24 genes that belong to the Claudin family, including Claudin 3 and Claudin 4, have been reported to be present on the human chromosome.

Both Claudin 3 and Claudin 4 are known to function as toxin receptors for the Clostridium perfringens enterotoxin (CPE). CPE binds to Claudin 3 and Claudin 4, and then produces a pore in the cell membrane by forming a large complex to cause cellular necrosis. Published Data show that administration of a sublethal amount of CPE to gallbladder cancer model mice produced by transplanting Claudin 3- and Claudin 4-expressing human tumor cells decreased tumor volume and increased survival rate (Non-Patent Documents 6 and 7). Although the possibility of clinically applying CPE to human has been suggested, such application has yet to be performed in practice due to its narrow therapeutic window between the dosage that shows drug efficacy and the dosage that causes lethal toxicity, and the concern regarding antigenicity of CPE in human.

Claudin 3 is a protein with four transmembrane regions, and has a structure that exposes two peptide loops to the outside of the cell. As shown below, the polypeptide portions which constitute the peptide sequences predicted to be the extracellular loops consist of only 51 amino acid residues (loop 1) and 23 amino acid residues (loop 2).

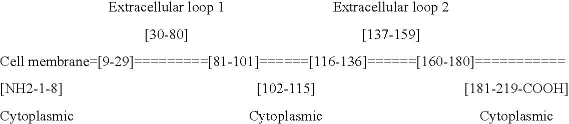

Since the sequence identity of the Claudin family is high among animal species, it was extremely difficult to obtain antibodies that recognize the extracellular domains by general immunization methods. Furthermore, since molecules that belong to the Claudin family have similar structures to each other, methods for obtaining an antibody that specifically recognizes a member of the Claudin family have not been established (Non-patent Document 8).

For antibodies that recognize Claudin 3 expressed on cells, only Non-patent Document 3 is available, which reports the isolation of polyclonal antibodies obtained by immunizing chickens with a partial peptide of Claudin 3 and then performing affinity purification using the peptide. To date, there is no report on examples of isolation of monoclonal antibodies that bind to the native structure of Claudin-3 expressed on cell surface or determination of their antitumor activity.
[Non-patent Document 1] Soini (2005) Histopathology 46, 551.
[Non-patent Document 2] Santin et al. (2005) Br. J. Cancer 92, 1561.
[Non-patent Document 3] Offner et al. (2005) Cancer Immunol. Immunother. 54, 431.
[Non-patent Document 4] Long et al. (2001) Cancer Res. 61, 7878.
[Non-patent Document 5] Rangel et al. (2003) Clin. Cancer Res. 9, 2567.
[Non-patent Document 6] Kominsky et al. (2004) Am. J. Path. 164, 1627.
[Non-patent Document 7] Santin et al. (2005) Cancer Res. 65, 4334.
[Non-patent Document 8] Hoevel et al. (2002) J. Cell. Physiol. 191, 60.

### DISCLOSURE OF THE INVENTION

### Problems to be Solved by the Invention

An objective of the present invention is to provide anti-Claudin 3 antibodies and uses thereof. More specifically, an objective is to provide novel anti-Claudin 3 antibodies, novel methods for treating cancer using anti-Claudin 3 antibodies, and novel cell proliferation inhibitors or anti-cancer agents containing an anti-Claudin 3 antibody.

### Means for Solving the Problems

The present inventors successfully obtained anti-Claudin 3 antibodies by immunizing mice with a Claudin 3 polypeptide-encoding DNA. Furthermore, the present inventors measured the activity of the thus-obtained antibodies to bind to the following Claudin family molecules, which are expressed on the cell surface:

the human Claudin 3 protein,

mouse Claudin 3 protein,

human Claudin 1 protein,

human Claudin 4 protein, and

human Claudin 6 protein.

As a result, the anti-Claudin 3 antibodies of the present invention were confirmed to have any or all of the following binding activities:

strong binding activity to the human Claudin 3 protein;

strong binding activity to the human and mouse Claudin 3 proteins; and

binding activity to the human Claudin 3 protein and human Claudin 4 protein.

Furthermore, the present inventors discovered that the obtained anti-Claudin 3 antibodies include antibodies that do not substantially show binding activity to synthetic peptides having the peptide sequences predicted to be the extracellular loops, but bind specifically to the human Claudin 3 protein expressed on the cell surface.

The present inventors also discovered that the obtained anti-Claudin 3 antibodies show binding activity to the MCF7 human breast cancer cell line endogenously expressing Claudin 3, and demonstrated that the antibodies are useful for diagnosis of various types of primary or metastatic cancer cells. Furthermore, the present inventors discovered that any or all of the various types of cancer tissues described below can be diagnosed using the anti-Claudin 3 antibodies:

various types of cancer tissues expressing a Claudin 3 protein;

various types of cancer tissues expressing a Claudin 4 protein; and

cancer tissues expressing both the Claudin 3 and Claudin 4 proteins.

Furthermore, the present inventors measured the complement-dependent cytotoxicity (CDC) activity of the anti-Claudin 3 antibodies against DG44 cells stably expressing the human Claudin 3 protein and the aforementioned MCF7 cells. The present inventors discovered that the anti-Claudin 3 antibodies of the present invention have CDC activity against both of these cells. The present inventors also measured the antibody-dependent cell-mediated cytotoxicity (ADCC) activity of the anti-Claudin 3 antibodies against MCF7 cells, and discovered that the anti-Claudin 3 antibodies of the present invention have ADCC activity against MCF7 cells.

From the above-mentioned findings, the present inventors discovered that anti-Claudin 3 antibodies are effective for diagnosing, preventing, or treating various types of primary or metastatic cancers, and completed the present invention.

The present invention provides monoclonal antibodies that bind to a Claudin 3 protein. The present invention also provides pharmaceutical compositions comprising an antibody that binds to a Claudin 3 protein as an active ingredient. The present invention also provides anticancer agents comprising an antibody that binds to a Claudin 3 protein as an active ingredient. Preferably, the antibodies that bind to a Claudin 3 protein have cytotoxic activity. In a preferred embodiment of the present invention, cancers that can be targeted for treatment are ovarian cancer, prostate cancer, breast cancer, uterine cancer, liver cancer, lung cancer, pancreatic cancer, stomach cancer, bladder cancer, and colon cancer. Breast cancer is particularly preferred. Anticancer agents containing an anti-Claudin 3 antibody of the present invention are useful for treating these cancers which are primary or metastatic cancers and have elevated expression of Claudin 3.

Furthermore, the present invention provides pharmaceutical compositions comprising an antibody that binds to a Claudin 3 protein and a pharmaceutically acceptable carrier. Pharmaceutical compositions of the present invention are useful for treating and/or preventing cancers that have elevated expression of Claudin 3. That is, the present invention relates to the use of an antibody that binds to a Claudin 3 protein for the production of pharmaceutical compositions for treating and/or preventing cancer.

In another embodiment, the present invention provides methods for inducing cell injury in cells that express a Claudin 3 protein by contacting Claudin 3-expressing cells with an antibody that binds to a Claudin 3 protein. The present invention also provides methods for suppressing proliferation of cells that express a Claudin 3 protein by contacting Claudin 3 protein-expressing cells with an antibody that binds to a Claudin 3 protein. The antibody that binds to a Claudin 3 protein preferably has cytotoxic activity. Cells that express a Claudin 3 protein are preferably cancer cells.

Furthermore, in another embodiment, the present invention provides antibodies that bind to a Claudin 3 protein and have cytotoxic activity in cells that express the Claudin 3 protein. Preferably, the cytotoxic activity is ADCC activity. Preferably, the cytotoxic activity is CDC activity. The present invention also provides antibodies to which a cytotoxic substance is bound. In the present invention, the cytotoxic substances that may be bound to the antibody include chemotherapeutic agents, radioisotopes, and toxic peptides. Preferably, in the present invention, an antibody itself has cytotoxic activity.

Furthermore, in another embodiment, the present invention provides methods for diagnosing cancer, which comprises detecting a Claudin 3 protein using an antibody that binds to the Claudin 3 protein. In the methods of the present invention, preferably, the extracellular region of a Claudin 3 protein is detected. Preferably, the methods of the present invention are carried out using an antibody that recognizes a Claudin 3 protein.

In another embodiment, the present invention provides methods for diagnosis of cancer which comprise the following steps of:

(a) collecting a sample from a subject; and
(b) using an antibody that binds to a Claudin 3 protein to detect the Claudin 3 protein contained in the collected sample.
In the present invention, any sample can be used as the above-mentioned sample as long as it can be collected from the subject. In one embodiment, blood sample collected from a subject is used. In another embodiment, samples collected surgically or by biopsy from a subject may be used. The methods of diagnosis can be used for any cancer as long as it is a cancer in which the target cancer cells express a Claudin 3 protein. Cancers that are preferred in the present invention are ovarian cancer, prostate cancer, breast cancer, uterine cancer, liver cancer, lung cancer, pancreatic cancer, stomach cancer, bladder cancer, and colon cancer. Breast cancer is particularly preferable. Based on the present invention, both primary and metastatic foci of these cancers can be diagnosed. In the present invention, the step of collecting a sample from a subject can also be expressed as the step of providing a sample collected from a subject.

Furthermore, in another embodiment, the present invention provides methods for diagnosis of cancer, which comprise the steps of: (1) administering to a subject a radioisotope-labeled antibody that binds to a Claudin 3 protein; and (2) detecting accumulation of the radioisotope. In a certain embodiment, the radioisotope is a positron-emitting nuclide. A preferred positron-emitting nuclide of the present invention can be selected, for example, from the group consisting of ¹¹C, ¹³N, ¹⁵O, ¹⁸F, ⁴⁵Ti, ⁵⁵Co, ⁶⁴Cu, ⁶⁶Ga, ⁶⁸Ga, ⁷⁶Br, ⁸⁹Zr, and ¹²⁴I.

Furthermore, in another embodiment, the present invention provides methods for diagnosis of cancer, which comprise detecting the expression of a gene encoding the Claudin 3 protein. Furthermore, in another embodiment, the present invention provides diagnostic agents and kits to be used in the diagnostic methods of the present invention.

More specifically, the present invention provides the following:

[1] a monoclonal antibody that binds to a Claudin 3 protein;
[2] the monoclonal antibody of [1], wherein the antibody binds to a protein expressed on the cell membrane and comprising the amino acid sequence of SEQ ID NO: 2;
[3] the monoclonal antibody of [2], wherein the antibody does not substantially cross-react with a peptide comprising the amino acid sequence of positions 30 to 80 or positions 137 to 159 in the amino acid sequence of SEQ ID NO: 2;
[4] the monoclonal antibody of any one of [1] to [3], wherein the antibody binds to a protein expressed on the cell membrane and comprising the amino acid sequence of SEQ ID NO: 4;
[5] the monoclonal antibody of any one of [1] to [3], wherein the antibody binds to a protein expressed on the cell membrane and comprising the amino acid sequence of SEQ ID NO: 8;
[6] an antibody that binds to a protein expressed on the cell membrane and comprising the amino acid sequence of any one of SEQ ID NOs: 2, 4, and 8, by recognizing a conformation formed by two extracellular loops of the protein;
[7] the antibody of [6], wherein the antibody does not substantially cross-react with a peptide comprising the amino acid sequence of positions 30 to 80 or positions 137 to 159 in the amino acid sequence of SEQ ID NO: 2;
[8] the antibody of [6] or [7], wherein the antibody is a monoclonal antibody;
[9] the antibody of any one of [1] to [8], wherein the antibody has cytotoxic activity;
[10] the antibody of [9], wherein the cytotoxic activity is ADCC activity;
[11] the antibody of [9], wherein the cytotoxic activity is CDC activity;
[12] the antibody of any one of [1] to [11], wherein a chemotherapeutic agent or a toxic peptide is bound to the antibody;
[13] an antibody that binds to a Claudin 3 protein, wherein a cytotoxic substance selected from the group consisting of a chemotherapeutic agent, toxic peptide, and radioisotope is bound to the antibody;
[14] the antibody of any one of [1] to [13], which is an antibody described in any of (1) to (61) below:
(1) an antibody comprising an H chain having the amino acid sequence of SEQ ID NO: 12 as CDR1, the amino acid sequence of SEQ ID NO: 14 as CDR2, and the amino acid sequence of SEQ ID NO: 16 as CDR3;
(2) an antibody comprising the H chain of (1), wherein the H chain has the amino acid sequence of positions 139 to 462 in the amino acid sequence of SEQ ID NO: 20 as CH;
(3) an antibody comprising the H chain of (1), wherein the H chain has the amino acid sequence of SEQ ID NO: 22 as CH;
(4) an antibody comprising an L chain having the amino acid sequence of SEQ ID NO: 24 as CDR1, the amino acid sequence of SEQ ID NO: 26 as CDR2, and the amino acid sequence of SEQ ID NO: 28 as CDR3;
(5) an antibody comprising the L chain of (4), wherein the L chain has the amino acid sequence of positions 135 to 240 in the amino acid sequence of SEQ ID NO: 32 as CL;
(6) an antibody comprising the L chain of (4), wherein the L chain has the amino acid sequence of SEQ ID NO: 34 as CL;
(7) an antibody comprising the H chain of (1) and the L chain of (4);
(8) an antibody comprising the H chain of (2) and the L chain of (5);
(9) an antibody comprising the H chain of (3) and the L chain of (6);
(10) an antibody having one or more amino acid substitutions, deletions, additions, and/or insertions in the antibody of any of (1) to (9), which has equivalent activity as the antibody of any of (1) to (9);
(11) an antibody comprising an H chain having the amino acid sequence of SEQ ID NO: 36 as CDR1, the amino acid sequence of SEQ ID NO: 38 as CDR2, and the amino acid sequence of SEQ ID NO: 40 as CDR3;
(12) an antibody comprising the H chain of (11), wherein the H chain has the amino acid sequence of positions 140 to 476 in the amino acid sequence of SEQ ID NO: 44 as CH;
(13) an antibody comprising the H chain of (11), wherein the H chain has the amino acid sequence of SEQ ID NO: 22 as CH;
(14) an antibody comprising an L chain having the amino acid sequence of SEQ ID NO: 46 as CDR1, the amino acid sequence of SEQ ID NO: 48 as CDR2, and the amino acid sequence of SEQ ID NO: 50 as CDR3;
(15) an antibody comprising the L chain of (14), wherein the L chain has the amino acid sequence of positions 133 to 238 in the amino acid sequence of SEQ ID NO: 54 as CL;
(16) an antibody comprising the L chain of (14), wherein the L chain has the amino acid sequence of SEQ ID NO: 34 as CL;
(17) an antibody comprising the H chain of (11) and the L chain of (14);
(18) an antibody comprising the H chain of (12) and the L chain of (15);
(19) an antibody comprising the H chain of (13) and the L chain of (16);
(20) an antibody having one or more amino acid substitutions, deletions, additions, and/or insertions in the antibody of any of (11) to (19), which has equivalent activity as the antibody of any of (11) to (19);
(21) an antibody comprising an H chain having the amino acid sequence of SEQ ID NO: 56 as CDR1, the amino acid sequence of SEQ ID NO: 58 as CDR2, and the amino acid sequence of SEQ ID NO: 60 as CDR3;
(22) an antibody comprising the H chain of (21), wherein the H chain has the amino acid sequence of positions 137 to 471 in the amino acid sequence of SEQ ID NO: 64 as CH;
(23) an antibody comprising the H chain of (21), wherein the H chain has the amino acid sequence of SEQ ID NO: 22 as CH;
(24) an antibody comprising an L chain having the amino acid sequence of SEQ ID NO: 66 as CDR1, the amino acid sequence of SEQ ID NO: 68 as CDR2, and the amino acid sequence of SEQ ID NO: 70 as CDR3;
(25) an antibody comprising the L chain of (24), wherein the L chain has the amino acid sequence of positions 133 to 238 in the amino acid sequence of SEQ ID NO: 74 as CL;
(26) an antibody comprising the L chain of (24), wherein the L chain has the amino acid sequence of SEQ ID NO: 34 as CL;
(27) an antibody comprising the H chain of (21) and the L chain of (24);
(28) an antibody comprising the H chain of (22) and the L chain of (25);
(29) an antibody comprising the H chain of (23) and the L chain of (26);
(30) an antibody having one or more amino acid substitutions, deletions, additions, and/or insertions in the antibody of any of (21) to (29), which has equivalent activity as the antibody of any of (21) to (29);
(31) an antibody comprising an H chain having the amino acid sequence of SEQ ID NO: 76 as CDR1, the amino acid sequence of SEQ ID NO: 78 as CDR2, and the amino acid sequence of SEQ ID NO: 80 as CDR3;
(32) an antibody comprising the H chain of (31), wherein the H chain has the amino acid sequence of positions 140 to 463 in the amino acid sequence of SEQ ID NO: 84 as CH;
(33) an antibody comprising the H chain of (31), wherein the H chain has the amino acid sequence of SEQ ID NO: 22 as CH;
(34) an antibody comprising an L chain having the amino acid sequence of SEQ ID NO: 86 as CDR1, the amino acid sequence of SEQ ID NO: 88 as CDR2, and the amino acid sequence of SEQ ID NO: 90 as CDR3;
(35) an antibody comprising the L chain of (34), wherein the L chain has the amino acid sequence of positions 133 to 238 in the amino acid sequence of SEQ ID NO: 94 as CL;
(36) an antibody comprising the L chain of (34), wherein the L chain has the amino acid sequence of SEQ ID NO: 34 as CL;
(37) an antibody comprising the H chain of (31) and the L chain of (34);
(38) an antibody comprising the H chain of (32) and the L chain of (35);
(39) an antibody comprising the H chain of (33) and the L chain of (36);
(40) an antibody having one or more amino acid substitutions, deletions, additions, and/or insertions in the antibody of any of (31) to (39), which has equivalent activity as the antibody of any of (31) to (39);
(41) an antibody comprising an H chain having the amino acid sequence of SEQ ID NO: 96 as CDR1, the amino acid sequence of SEQ ID NO: 98 as CDR2, and the amino acid sequence of SEQ ID NO: 100 as CDR3;
(42) an antibody comprising the H chain of (41), wherein the H chain has the amino acid sequence of positions 140 to 474 in the amino acid sequence of SEQ ID NO: 104 as CH;
(43) an antibody comprising the H chain of (41), wherein the H chain has the amino acid sequence of SEQ ID NO: 22 as CH;
(44) an antibody comprising an L chain having the amino acid sequence of SEQ ID NO: 106 as CDR1, the amino acid sequence of SEQ ID NO: 108 as CDR2, and the amino acid sequence of SEQ ID NO: 110 as CDR3;
(45) an antibody comprising the L chain of (44), wherein the L chain has the amino acid sequence of positions 133 to 238 in the amino acid sequence of SEQ ID NO: 114 as CL;
(46) an antibody comprising the L chain of (44), wherein the L chain has the amino acid sequence of SEQ ID NO: 34 as CL;
(47) an antibody comprising the H chain of (41) and the L chain of (44);
(48) an antibody comprising the H chain of (42) and the L chain of (45);
(49) an antibody comprising the H chain of (43) and the L chain of (46);
(50) an antibody having one or more amino acid substitutions, deletions, additions, and/or insertions in the antibody of any of (41) to (49), which has equivalent activity as the antibody of any of (41) to (49);
(51) an antibody comprising an H chain having the amino acid sequence of SEQ ID NO: 167 as CDR1, the amino acid sequence of SEQ ID NO: 169 as CDR2, and the amino acid sequence of SEQ ID NO: 171 as CDR3;
(52) an antibody comprising the H chain of (51), wherein the H chain has the amino acid sequence of positions 118 to 447 in the amino acid sequence of SEQ ID NO: 173 as CH;
(53) an antibody comprising the H chain of (51), wherein the H chain has the amino acid sequence of SEQ ID NO: 22 as CH;
(54) an antibody comprising an L chain having the amino acid sequence of SEQ ID NO: 179 as CDR1, the amino acid sequence of SEQ ID NO: 181 as CDR2, and the amino acid sequence of SEQ ID NO: 183 as CDR3;
(55) an antibody comprising the L chain of (54), wherein the L chain has the amino acid sequence of positions 113 to 218 in the amino acid sequence of SEQ ID NO: 185 as CL;
(56) an antibody comprising the L chain of (54), wherein the L chain has the amino acid sequence of SEQ ID NO: 34 as CL;
(57) an antibody comprising the H chain of (51) and the L chain of (54);
(58) an antibody comprising the H chain of (52) and the L chain of (55);
(59) an antibody comprising the H chain of (53) and the L chain of (56);
(60) an antibody having one or more amino acid substitutions, deletions, additions, and/or insertions in the antibody of any of (51) to (59), which has equivalent activity as the antibody of any of (51) to (59);
(61) an antibody that binds to the same epitope as the Claudin 3 protein epitope bound by the antibody of any of (1) to (60);
[15] a method for diagnosis of cancer, comprising the step of binding the antibody of any one of [1] to [14] to a Claudin 3 protein;
[16] a method for diagnosis of cancer, comprising the steps of:
(a) collecting a sample from a subject; and
(b) detecting a Claudin 3 protein contained in the collected sample using an antibody that binds to the Claudin 3 protein;
[17] a method for diagnosis of cancer, comprising the steps of: (1) administering to a subject a radioisotope-labeled antibody that binds to a Claudin 3 protein; and (2) detecting accumulation of said radioisotope;
[18] the diagnostic method of [17], wherein the radioisotope is a positron-emitting nuclide;
[19] the diagnostic method of [18], wherein the positron-emitting nuclide is any nuclide selected from the group consisting of ¹¹C, ¹³N, ¹⁵O, ¹⁸F, ⁴⁵Ti, ⁵⁵Co, ⁶⁴Cu, ⁶⁶Ga, ⁶⁸Ga, ⁷⁶Br, ⁸⁹Zr, and ¹²⁴I;
[20] the diagnostic method of any one of [15] to [19], wherein the cancer is any cancer selected from the group consisting of ovarian cancer, prostate cancer, breast cancer, uterine cancer, liver cancer, lung cancer, pancreatic cancer, stomach cancer, bladder cancer, and colon cancer;
[21] the diagnostic method of [20], wherein the cancer is primary cancer;
[22] the diagnostic method of [20], wherein the cancer is metastatic cancer;
[23] a diagnostic agent for use in a method of cancer diagnosis, which comprises an antibody that binds to a Claudin 3 protein;
[24] a kit for use in a method of cancer diagnosis, which comprises an antibody that binds to a Claudin 3 protein, and a biological sample comprising a Claudin 3 protein;
[25] a pharmaceutical composition comprising an antibody that binds to a Claudin 3 protein as an active ingredient;
[26] a cell proliferation inhibitor comprising an antibody that binds to a Claudin 3 protein as an active ingredient;
[27] an anticancer agent comprising an antibody that binds to a Claudin 3 protein as an active ingredient;
[28] the anticancer agent of [27], wherein the cancer is any cancer selected from the group consisting of ovarian cancer, prostate cancer, breast cancer, uterine cancer, liver cancer, lung cancer, pancreatic cancer, stomach cancer, bladder cancer, and colon cancer;
[29] the anticancer agent of [28], wherein the cancer is primary cancer;
[30] the anticancer agent of [28], wherein the cancer is metastatic cancer;
[31] the anticancer agent of [28], wherein said antibody is the antibody of any one of [1] to [14];
[32] use of an antibody that binds to a Claudin 3 protein in the production of a cell proliferation inhibitor;
[33] use of an antibody that binds to a Claudin 3 protein in the production of an anticancer agent;
[34] the use of [33], wherein the cancer is any cancer selected from the group consisting of ovarian cancer, prostate cancer, breast cancer, uterine cancer, liver cancer, lung cancer, pancreatic cancer, stomach cancer, bladder cancer, and colon cancer;
[35] the use of [33], wherein the cancer is primary cancer;
[36] the use of [33], wherein the cancer is metastatic cancer;
[37] the use of [32] or [33], wherein the antibody is the antibody of any one of [1] to [14];
[38] a method of suppressing cell proliferation, which comprises the step of administering to a subject an antibody that binds to a Claudin 3 protein;
[39] a method of preventing or treating cancer, which comprises the step of administering to a subject an antibody that binds to a Claudin 3 protein;
[40] the method of [39], wherein the cancer is any cancer selected from the group consisting of ovarian cancer, prostate cancer, breast cancer, uterine cancer, liver cancer, lung cancer, pancreatic cancer, stomach cancer, bladder cancer, and colon cancer;
[41] the method of [39], wherein the cancer is primary cancer;
[42] the method of [39], wherein the cancer is metastatic cancer;
[43] the method of [38] or [39], wherein the antibody is the antibody of any one of [1] to [14];
[44] a method of inducing cell injury in a Claudin 3 protein-expressing cell, which comprises the step of contacting the Claudin 3 protein-expressing cell with an antibody that binds to a Claudin 3 protein;
[45] a method of suppressing proliferation of a Claudin 3 protein-expressing cell, which comprises the step of contacting the Claudin 3 protein-expressing cell with an antibody that binds to a Claudin 3 protein;
[46] the method of [44] or [45], wherein the Claudin 3 protein-expressing cell is a cancer cell;
[47] the method of any one of [44] to [46], wherein the antibody is an antibody having cytotoxic activity;
[48] the method of any one of [44] to [46], wherein the antibody is the antibody of any one of [1] to [14];
[49] an antibody that binds to a Claudin 3 protein for use in a method of cancer treatment;
[50] the antibody of [49], wherein the cancer is any cancer selected from the group consisting of ovarian cancer, prostate cancer, breast cancer, uterine cancer, liver cancer, lung cancer, pancreatic cancer, stomach cancer, bladder cancer, and colon cancer;
[51] the antibody of [50], wherein the cancer is primary cancer;
[52] the antibody of [50], wherein the cancer is metastatic cancer; and
[53] the antibody of [50], which is the antibody of any one of [1] to [14].

### BRIEF DESCRIPTION OF THE DRAWINGS

FIG. 1 shows a dendrogram and alignment of the extracellular loops of Claudin 3 and other members of human Claudin family.

FIG. 2 shows an amino acid sequence alignment of human and mouse Claudin 3. The underlines indicate the putative transmembrane regions, and the boxes indicate the putative extracellular regions.

FIG. 3 shows differences in the binding reactivity of anti-Claudin 3 antibodies for Claudin 3-expressing DG44 cells and Ba/F3 cells or the MCF7 breast cancer cell line: (A) Ba/F3 cells, (B) the MCF7 breast cancer cell line. The X geometric mean values (relative fluorescence intensity values) are shown on the vertical and horizontal axes.

FIG. 4 shows the affinity of anti-Claudin 3 monoclonal antibodies for the Claudin 3 extracellular loop peptide regions. The vertical axis indicates the absorbance at 405 nm (reference wavelength of 655 nm), and the horizontal axis indicates the anti-Claudin 3 antibody concentration (ng/mL).

FIG. 5 shows the antigen-dependent induction of cytotoxic activity by anti-Claudin 3 antibodies. The vertical axis indicates the percentage increase in dead cells by complement addition.

FIG. 6 shows the anti-Claudin 3 antibody-mediated induction of complement-dependent cytotoxic activity against MCF7 cells. The vertical axis indicates the specific chromium release rate (%).

FIG. 7 shows the anti-Claudin 3 antibody-mediated induction of antibody-dependent cell-mediated cytotoxic activity against MCF7 cells. The vertical axis indicates the specific chromium release rate (%).

FIG. 8 shows, by flow cytometry, the specific binding of recombinant chimeric antibodies to cells forced to express Claudin 3. The vertical axis indicates cell count (fluorescence coefficient), and the horizontal axis indicates fluorescence intensity.

FIG. 9-1 presents graphs showing the results of FACS analysis of the binding activity of various anti-Claudin 3 antibodies to the recombinant cells below (results for the CDN1, CDN2, and CDN3 monoclonal antibodies). “CLD3/3”: wild-type Claudin 3-expressing cells; “1/3”: CLD1/3 chimeric protein-expressing cells; “3/1”: CLD3/1 chimeric protein-expressing cells; and “Ba/F3”: Ba/F3 cells as a parental cell. The X axis indicates fluorescence intensity, and the Y axis indicates the relative cell count at each fluorescence intensity.

FIG. 9-2 is the continuation of FIG. 9-1 (results for the CDN4, CDN5, and CDN7 anti-Claudin 3 monoclonal antibodies).

FIG. 9-3 is the continuation of FIG. 9-2 (results for the CDN8, CDN16, and CDN17 anti-Claudin 3 monoclonal antibodies).

FIG. 9-4 is the continuation of FIG. 9-3 (results for the CDN24, CDN27, and CDN28 anti-Claudin 3 monoclonal antibodies).

FIG. 9-5 is the continuation of FIG. 9-4 (results for the CDN29, CDN30, and CDN31 anti-Claudin 3 monoclonal antibodies).

FIG. 9-6 is the continuation of FIG. 9-5 (results for the CDN32, CDN33, and CDN35 anti-Claudin 3 monoclonal antibodies).

FIG. 9-7 is the continuation of FIG. 9-6 (results for the CDN36, CDN37, and CDN38 anti-Claudin 3 monoclonal antibodies).

FIG. 9-8 is the continuation of FIG. 9-7 (results for anti-Claudin 3 antiserum diluted 200-fold, anti-Claudin 3 antiserum diluted 1000-fold, and the negative control without antibody addition).

FIG. 10 is a graph showing the proportion of cells having a relative fluorescence intensity of 100 or more with respect to total cells. The fluorescence intensity is shown on the horizontal axis of the histogram plots of FIGS. 9-1 to 9-7.

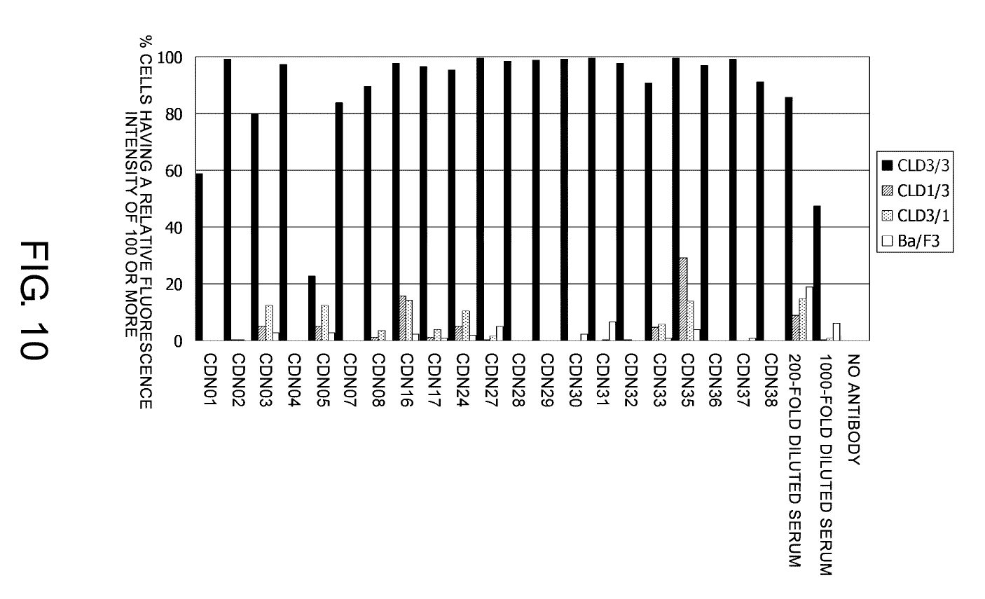

FIG. 11 shows, by flow cytometry, the binding of recombinant chimeric antibodies to cells forced to express Claudin 3. The vertical axis indicates cell number (fluorescence coefficient), and the horizontal axis indicates fluorescence intensity. The black solid line and the grey dotted line represent the fluorescence intensity distribution of cells with and without addition of a chimeric antibody, respectively.

FIG. 12 is a graph showing suppression of the proliferation of MCF7 cells by anti-Claudin 3 antibodies in a soft agar colony formation/MTT hybrid assay.

FIG. 13 presents photographs showing suppression of the cell motility by addition of an anti-Claudin 3 antibody (CDN04) in a wound-healing assay. Cells in the region between the dashed lines are the cells that migrated to the scratched site.

### MODE FOR CARRYING OUT THE INVENTION

Claudin 3 is a protein that belongs to the Claudin family, and is a structural protein that regulates intercellular permeability. The amino acid sequence of human Claudin 3 and a nucleotide sequence encoding the protein are shown in SEQ ID NOs: 2 and 1, respectively (GenBank Accession No. NM_{—}001306). In the present invention, the Claudin 3 proteins recognized by the monoclonal antibodies are proteins that maintain the native conformation of a Claudin 3 protein. Furthermore, the monoclonal antibodies of the present invention bind to Claudin 3 preferably by recognizing its extracellular regions. Positions 30 to 80 in the amino acid sequence of SEQ ID NO: 2 (loop 1) and positions 137 to 159 in the amino acid sequence of SEQ ID NO: 2 (loop 2) correspond to the extracellular regions of the Claudin 3 protein.

As long as polypeptides containing the amino acid sequences of these extracellular regions maintain the cell-surface conformation of Claudin 3, monoclonal antibodies of the present invention can recognize the polypeptides. The monoclonal antibodies of the present invention preferably bind to polypeptides that maintain the native conformation of Claudin 3 expressed on the cell surface. Whether polypeptides maintain the native conformation of Claudin 3 can be checked, for example, as follows. When the immunological binding between the monoclonal antibodies of the present invention and cells expressing Claudin 3 on the cell surface is inhibited by certain polypeptides, it can be confirmed that these polypeptides maintain the conformation of the extracellular regions of naturally occurring Claudin 3.

Particularly, in a preferred embodiment of the present invention, monoclonal antibodies of the present invention recognize epitopes comprising the conformation formed by the two extracellular loop regions of Claudin 3. In the present invention, the epitopes comprising the conformation includes a structure formed by interactions within one polypeptide chain or interactions among multiple peptide chains. Such epitopes are also called “conformational epitopes”. For example, an epitope formed by the interaction between the two loops constituting the extracellular domains of Claudin 3 is included in the epitopes comprising the conformation of the present invention. That is, monoclonal antibodies of the present invention preferably recognize a conformational epitope formed by the two extracellular loops of Claudin 3.

Recognition of a conformational epitope by a monoclonal antibody can be confirmed, for example, as follows. For example, a linear peptide comprising the amino acid sequences constituting the extracellular loops of Claudin 3 is synthesized. Such a peptide can be synthesized chemically. Alternatively, the peptide can be obtained by a genetic engineering method using the regions in a Claudin 3 cDNA that encode the amino acid sequences corresponding to the loop portions. Then, the binding between a test antibody and the linear peptide comprising the amino acid sequences constituting the loop portions is evaluated. For example, the activity of a test antibody to bind to the linear peptide can be evaluated by ELISA using an immobilized form of the peptide as antigen. Alternatively, the activity to bind to the linear peptide can be assessed based on the level of inhibition of binding between a test antibody and Claudin 3-expressing cells by the linear peptide. These experiments can evaluate the activity of a test antibody to bind to the linear peptide.

In the present invention, when an antibody that binds to Claudin 3-expressing cells also has activity to bind to the linear peptide, “the antibody has cross-reactivity to the linear peptide”. In the present invention, preferred monoclonal antibodies do not substantially have cross-reactivity to the linear peptide comprising the amino acid sequences constituting the extracellular loops of Claudin 3. “Not substantially having cross-reactivity to the linear peptide” means that the activity of an antibody to bind to the linear peptide is, for example, 50% or less, generally 30% or less, or preferably 20% or less, as compared to the activity of the antibody to bind to Claudin 3-expressing cells.

Alternatively, recognition of a conformational epitope by a monoclonal antibody of the present invention can be confirmed as follows. Cells that express a chimeric molecule produced by linking one of the two extracellular loops of Claudin 3 with the other extracellular loop of a Claudin 3-like molecule are prepared. For example, human Claudin 1 can be used as a Claudin 3-like molecule. More specifically, cells forced to express the following chimeric molecules are produced.

| | 3/1 chimera | 1/3 chimera | Native |
|---|---|---|---|
| Loop 1 | Claudin 3 | Claudin 1 | Claudin 3 |
| Loop 2 | Claudin 1 | Claudin 3 | Claudin 3 |

A test antibody is contacted with these forced expression cells. If a monoclonal antibody has a lower activity of binding to both 3/1 chimera-expressing cells and 1/3 chimera-expressing cells, as compared to binding to native-type Claudin 3-expressing cells, the monoclonal body is an antibody that recognizes a conformational epitope of Claudin 3. In an embodiment, for example, a monoclonal antibody that recognizes an epitope formed by interactions between the two loops constituting the extracellular domains of Claudin 3 is one of the preferable antibodies that recognize a conformational epitope of Claudin 3 of the present invention. More specifically, a preferred monoclonal antibody of the present invention binds strongly to cells forced to express human Claudin 3, but does not substantially bind to either 3/1 chimera-expressing cells or 1/3 chimera-expressing cells. Herein, “not substantially binding” refers to a binding activity that is 80% or less, normally 50% or less, preferably 30% or less, or particularly preferably 15% or less compared to the activity of binding to human Claudin 3-expressing cells.

Examples of methods for evaluating the binding activity of an antibody to a cell include the method described on pages 359-420 of “Antibodies A Laboratory Manual” (Ed Harlow, David Lane, Cold Spring Harbor Laboratory, 1988). That is, the evaluation can be performed by the ELISA or fluorescence activated cell sorting (FACS) method using the cell as antigen.

In the ELISA format, the binding activity of an antibody to a cell is evaluated quantitatively by comparing the signal levels produced by enzymatic reaction. More specifically, a test antibody is added to ELISA plates on which the respective forced expression cells are immobilized, and the antibody bound to the cells is detected using an enzyme-labeled antibody that recognizes the test antibody. In FACS, the activity of binding to cells can be compared by producing a dilution series of a test antibody, and determining the binding titer of the antibody towards the respective forced expression cells.

The binding of an antibody to antigens expressed on the surface of cells suspended in a buffer solution or such can be detected by a flow cytometer. Known flow cytometers include the following instruments:

### FACSCanto™ II

### FACSAria™

### FACSArray™

### FACSVantage™ SE

FACSCalibur™ (all of the above are trade names of BD Biosciences)

### EPICS ALTRA HyPerSort

### Cytomics FC 500

### EPICS XL-MCL ADC EPICS XL ADC

Cell Lab Quanta/Cell Lab Quanta SC (all of the above are trade names of Beckman Coulter).

An example of a preferred method for measuring the activity of a test Claudin 3 antibody to bind to an antigen is set forth below. Staining is performed using an FITC-labeled secondary antibody that recognizes the test antibody which has been reacted with Claudin 3-expressing cells. The concentration of a test antibody used can be adjusted to a desired concentration by appropriately diluting the antibody in a suitable buffer. For example, the antibody can be used at any concentration between 10 μg/mL and 10 ng/mL. The fluorescence intensity and cell count are determined using FACSCalibur (BD). The amount of antibody bound to the cells is reflected in the fluorescence intensity, i.e., the geometric mean value, which is obtained by an analysis using the CellQuest Software (BD). That is, the binding activity of an antibody, which is represented by the amount of antibody bound, can be determined by obtaining the geometric mean value.

In the present method, for example, “not substantially binding to 3/1 chimera-expressing cells” can be determined by the following method. First, a test antibody bound to the aforementioned 3/1 chimeric molecule-expressing cells is stained with a secondary antibody. For example, if the test antibody is a mouse antibody, an FITC-labeled anti-mouse immunoglobulin antibody can be used as the secondary antibody. Then, the fluorescence intensity of the cells is detected. When FACSCalibur is used as the flow cytometer for fluorescence detection, the obtained fluorescence intensity can be analyzed using the CellQuest Software. The rate of increase in fluorescence intensity as a result of the binding of a test antibody can be determined by using the equation below to calculate the Δ Geo-Mean ratio from the geometric mean values in the presence or absence of the test antibody. Δ Geo-Mean=geometric mean (in the presence of a test antibody)/geometric mean (in the absence of the test antibody)

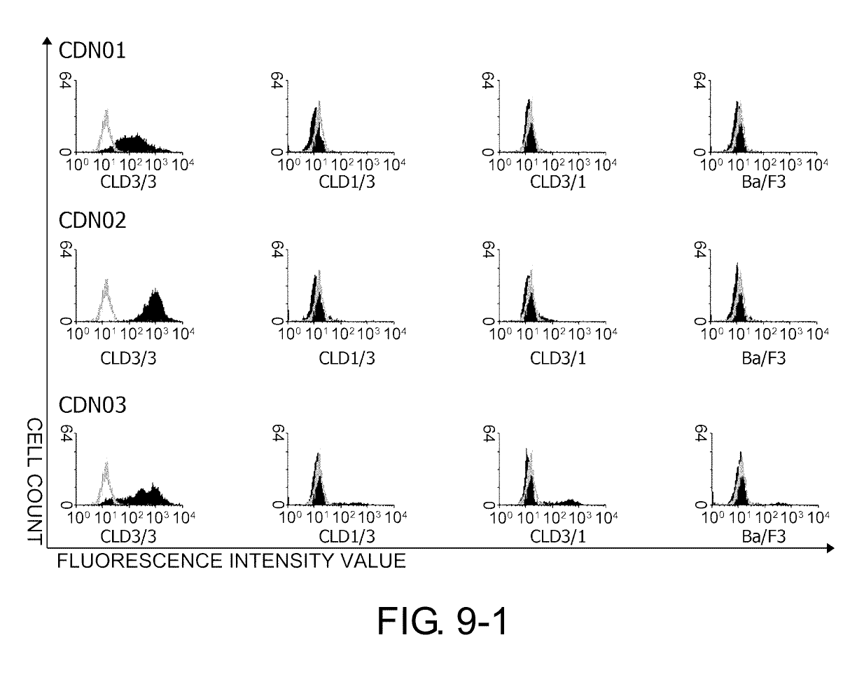

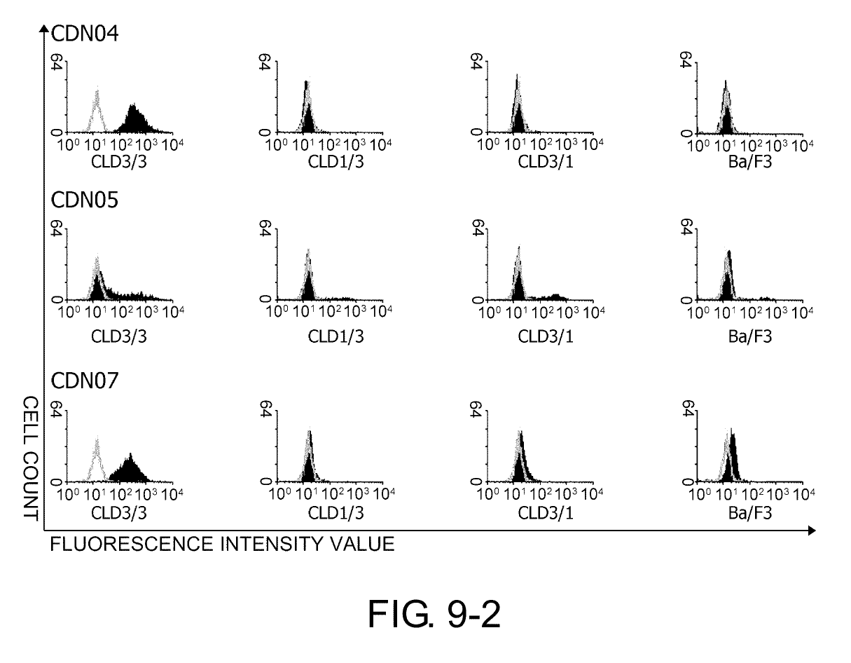

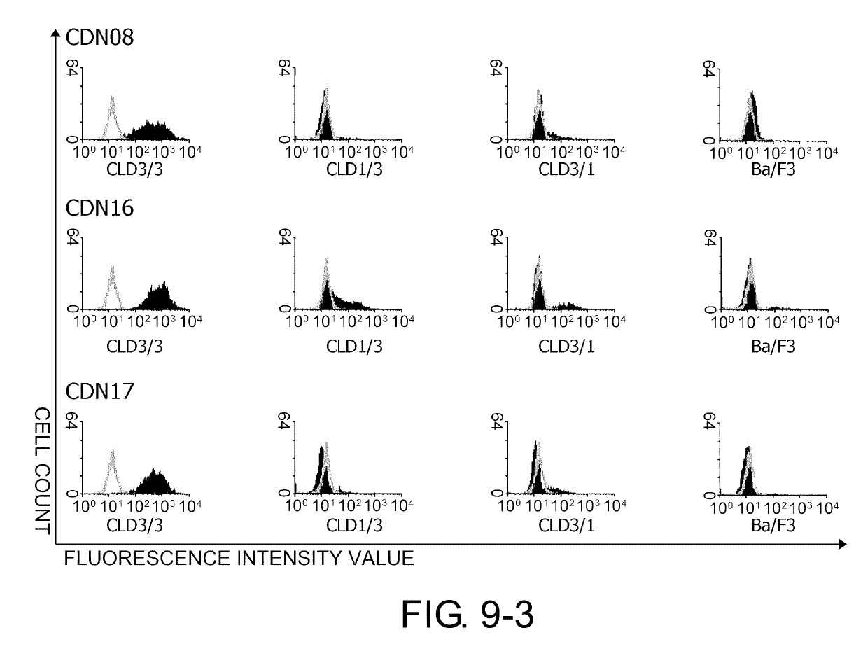

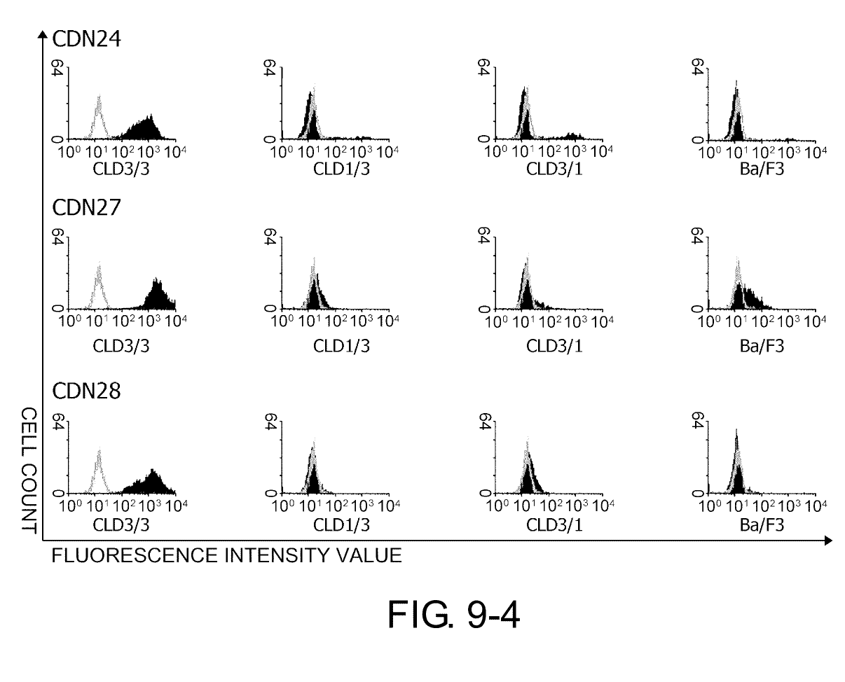

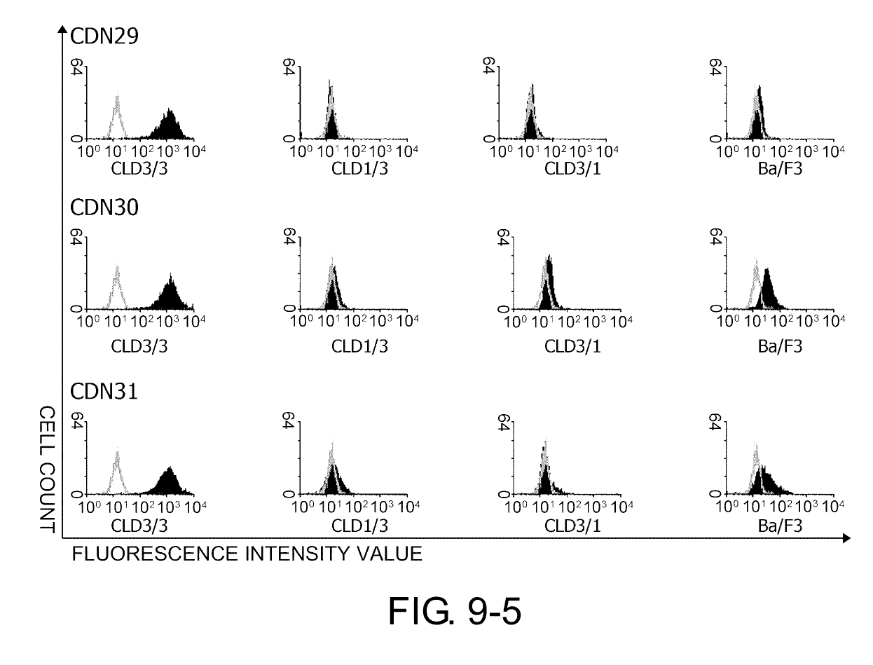

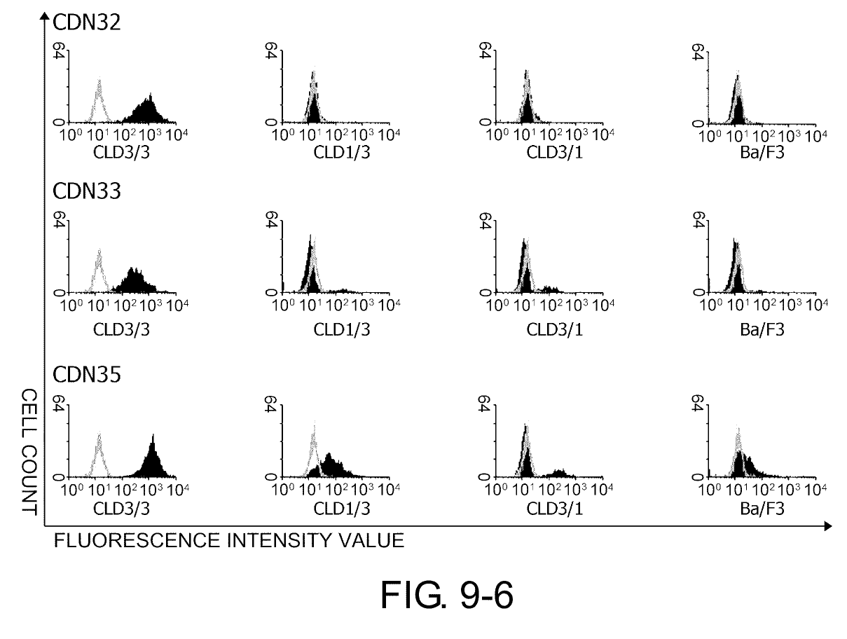

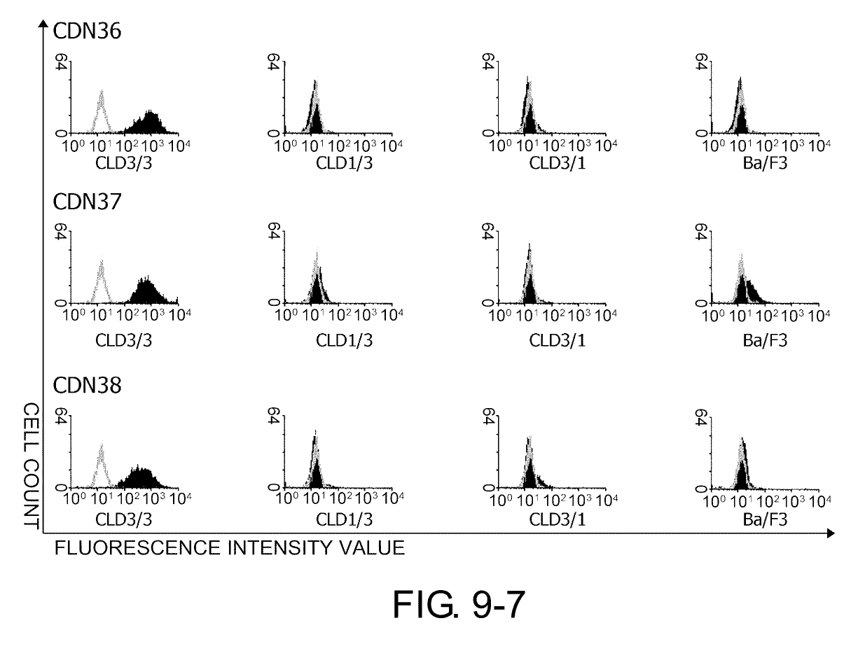

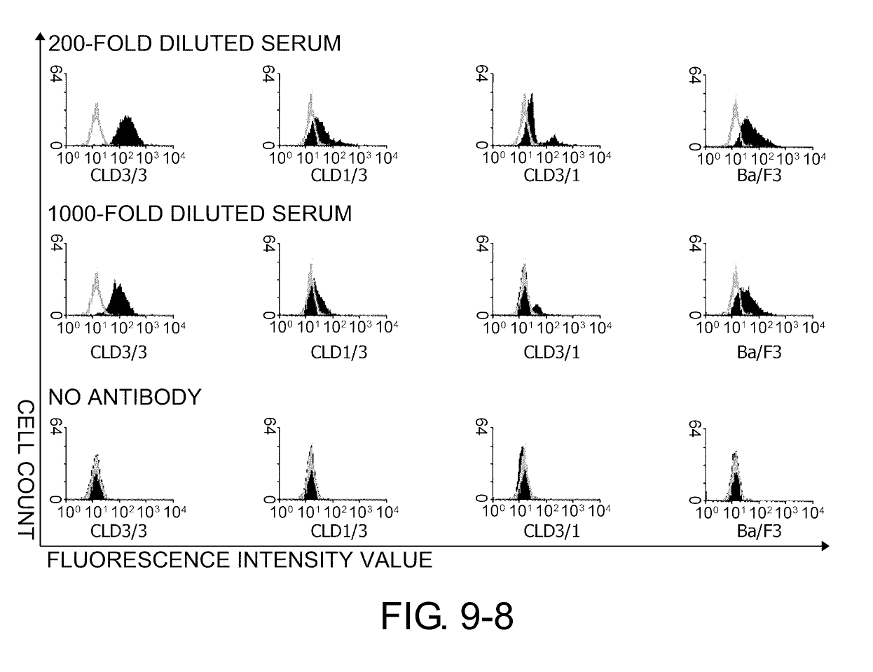

The geometric mean ratio that reflects the level of binding of a test antibody to 3/1 chimeric molecule-expressing cells (Δ Geo-Mean for 3/1 chimeric molecule) obtained by the analysis is compared with the Δ Geo-Mean ratio that reflects the level of binding of the test antibody to Claudin 3-expressing cells. In this case, it is particularly preferable to adjust the test antibody concentrations used for determining the Δ Geo-Mean ratios for 3/1 chimeric molecule-expressing cells and Claudin 3-expressing cells, to be identical or substantially identical to each other. A monoclonal antibody that has been confirmed in advance to recognize a conformational epitope of Claudin 3 can be used as a control antibody. For example, the monoclonal antibodies of the present invention shown below and in FIG. 9 can be used as antibodies that recognize a conformational epitope of Claudin 3:

CDN01, CDN02, CDN03, CDN04, CDN05, CDN07, CDN08,

CDN16, CDN17, CDN24, CDN27, CDN28, CDN29,

CDN30, CDN31, CDN32, CDN33, CDN35, CDN36, CDN37, and CDN38.

In the present invention, if the Δ Geo-Mean ratio of a test antibody for 3/1 chimeric molecule-expressing cells is smaller than at least 80%, preferably 50%, more preferably 30%, and particularly preferably 15% of the Δ Geo-Mean ratio of the test antibody for Claudin 3-expressing cells, it is determined that the test antibody “does not substantially bind to 3/1 chimeric molecule-expressing cells”. The equation for determining the geometric mean values is described in the CellQuest Software User's Guide (BD Biosciences). The activity to bind to 1/3 chimeric molecule-expressing cells can be evaluated similarly. For preparation of cells expressing the 3/1 chimera molecule, 1/3 chimera molecule, or Claudin 3 for evaluating the binding activity, it is preferable to use a common known expression vector and host cell, and to keep the expression levels in the respective cells the same.

Monoclonal antibodies of the present invention include monoclonal antibodies that bind to human Claudin 3-expressing cells, and whose activity of binding to cells expressing a chimeric molecule comprising a human Claudin 3 extracellular loop and a human Claudin 1 extracellular loop is lower than the activity of binding to the aforementioned human Claudin 3-expressing cells. Alternatively, preferred monoclonal antibodies of the present invention include monoclonal antibodies that bind to human Claudin 3-expressing cells, but do not substantially bind to cells expressing a chimeric molecule comprising a human Claudin 3 extracellular loop and a human Claudin 1 extracellular loop.

### Production of Anti-Claudin 3 Monoclonal Antibodies:

Monoclonal antibodies of the present invention can be obtained by DNA immunization. Mammal-derived monoclonal antibodies are particularly preferred as anti-Claudin 3 monoclonal antibodies of the present invention. The mammal-derived monoclonal antibodies include those produced by hybridomas, and those produced by hosts transformed with an expression vector containing an antibody gene using genetic engineering methods.

Monoclonal antibody-producing hybridomas of the present invention can be produced by DNA immunization as follows. DNA immunization is a method for providing immune stimulation by administering to an animal to be immunized, a vector DNA constructed so that a gene encoding an antigenic protein can be expressed in the immunized animal, and then expressing the immunogen in the body of the immunized animal. Compared to conventional immunization methods in which a protein antigen is administered, the following advantages can be expected from DNA immunization.

Immune stimulation can be provided while maintaining the structure of a membrane protein such as Claudin 3.

There is no need to purify an immunogen.

On the other hand, it is difficult to combine DNA immunization with the use of a means for immune stimulation such as an adjuvant. The identity between human and mouse Claudin 3 is particularly high in loop 1, which constitutes the extracellular region, 46/51. It was an unexpected achievement to obtain monoclonal antibodies that recognize proteins sharing such high interspecies identity by DNA immunization.

To obtain monoclonal antibodies of the present invention by DNA immunization, a DNA for expressing a Claudin 3 protein is administered to an animal to be immunized. A DNA encoding Claudin 3 can be synthesized by known methods such as PCR. The obtained DNA is inserted into a suitable expression vector, and then administered to an animal for immunization. Commercially available expression vectors such as pcDNA3.1 may be used as an expression vector. Conventional methods can be used to administer a vector to an organism. For example, gold particles adsorbed with an expression vector are shot into cells using a gene gun for DNA immunization.

According to the findings of the present inventors, hybridomas that produce Claudin 3-binding antibodies could not be obtained efficiently from mice immunized by intraperitoneal administration of cells forced to express Claudin 3. On the other hand, hybridomas that produce Claudin 3-binding antibodies could be obtained efficiently from mice immunized using DNA immunization. In particular, the hybridomas of interest could be readily obtained from mice to which cells forced to express Claudin 3 were administered after DNA immunization. That is, in a preferred method for obtaining the monoclonal antibodies of the present invention, a booster immunization using Claudin 3-expressing cells is performed after DNA immunization.

In the present invention, any non-human animal can be used as an animal for immunization. To obtain monoclonal antibodies by the cell fusion method, the animal to be immunized is preferably selected in consideration of its compatibility with the parental cell used for cell fusion. Generally, a rodent is preferred as the animal for immunization. More specifically, mice, rats, hamsters, or rabbits can be used as an animal for immunization. Alternatively, monkeys and such may be used as an animal for immunization.

Animals are immunized as described above. After confirming the increase in the titer of an antibody of interest in the serum, antibody-producing cells are collected from the immunized animals, and cloned. Preferred immunocytes (antibody-producing cells) are splenocytes.

A mammalian myeloma cell is used as a cell to be fused with the above-mentioned immunocyte. The myeloma cells preferably comprise a suitable selection marker for screening. A selection marker confers characteristics to cells for their survival (or failure to survive) under a specific culturing condition. Hypoxanthine-guanine phosphoribosyltransferase deficiency (hereinafter abbreviated as HGPRT deficiency), and thymidine kinase deficiency (hereinafter abbreviated as TK deficiency) are known as selection markers. Cells having HGPRT or TK deficiency have hypoxanthine-aminopterin-thymidine sensitivity (hereinafter abbreviated as HAT sensitivity). HAT-sensitive cells cannot carry out DNA synthesis in a HAT selection medium, and are thus killed. However, when the cells are fused with normal cells, they can continue to synthesize DNA using the salvage pathway of the normal cells, and therefore they can grow in the HAT selection medium.

HGPRT-deficient and TK-deficient cells can be selected in a medium containing 6-thioguanine or 8-azaguanine (hereinafter abbreviated as 8AG), and 5′-bromodeoxyuridine, respectively. Normal cells are killed since they incorporate these pyrimidine analogs into their DNA. On the other hand, cells that are deficient in these enzymes can survive in the selection medium, since they cannot incorporate these pyrimidine analogs. Alternatively, a selection marker referred to as G418 resistance provides resistance to 2-deoxystreptamine-type antibiotics (gentamycin analogs) from the neomycin-resistance gene. Various types of myeloma cells that are suitable for cell fusion are known. For example, myeloma cells including the following cells can be used to produce the monoclonal antibodies of the present invention:
P3 (P3x63Ag8.653) (J. Immunol. (1979) 123, 1548-1550);
P3x63Ag8U.1 (Current Topics in Microbiology and Immunology (1978) 81, 1-7);
NS-1 (Kohler. G. and Milstein, C. Eur. J. Immunol. (1976) 6, 511-519);
MPC-11 (Margulies. D. H. et al., Cell (1976) 8, 405-415);
SP2/0 (Shulman, M. et al., Nature (1978) 276, 269-270);
FO (de St. Groth, S. F. et al., J. Immunol. Methods (1980) 35, 1-21);
S194 (Trowbridge, I. S. J. Exp. Med. (1978) 148, 313-323); and
R210 (Galfre, G. et al., Nature (1979) 277, 131-133).

Cell fusion of the above-mentioned immunocytes with myeloma cells is essentially performed according to a known method, for example, the method of Kohler and Milstein et al. (Kohler. G. and Milstein, C., Methods Enzymol. (1981) 73, 3-46).

More specifically, the above-mentioned cell fusion can be performed in a standard nutritional culture medium in the presence of, for example, a cell-fusion accelerator. A cell-fusion accelerator may be, for example, polyethylene glycol (PEG), Sendai virus (HVJ), or the like. If desired, an auxiliary agent such as dimethylsulfoxide can be added to further enhance fusion efficiency.

The ratio of immunocytes to myeloma cells used can be established at one's discretion. For example, the number of immunocytes is preferably set to one to ten times of that of myeloma cells. As a medium to be used for the above-mentioned cell fusion, for example, RPMI1640 medium and MEM medium, which are appropriate for the growth of the above-mentioned myeloma cell line, or other standard media that are used for this type of cell culture can be used. Moreover, a serum supplement solution such as fetal calf serum (FCS) can be added to the media.

Cell fusion is performed by thoroughly mixing predetermined amounts of the above-mentioned immunocytes and myeloma cells in the above-mentioned medium, adding and mixing with a PEG solution pre-heated to approximately 37° C., so as to form the desired fused cells (hybridomas). In the cell fusion method, for example, PEG with an average molecular weight of approximately 1000 to 6000 can generally be added at a concentration of 30 to 60% (w/v). Subsequently, the agent for cell fusion or the like which is unfavorable for the growth of hybridomas can be removed by successively adding an appropriate medium such as those listed above, removing the supernatant after centrifugation, and repeating these steps.

Hybridomas obtained in this manner can be selected using a selection medium appropriate for the selection markers carried by myelomas used for cell fusion. For example, cells that have HGPRT and TK deficiencies can be selected by culturing them in a HAT medium (a medium containing hypoxanthine, aminopterin, and thymidine). More specifically, when HAT-sensitive myeloma cells are used for cell fusion, cells that successfully fuse with normal cells can be selectively grown in the HAT medium. Culturing using the above-mentioned HAT medium is continued for a sufficient period of time to kill the cells other than the hybridoma of interest (non-fused cells). More specifically, the hybridoma of interest can be selected, typically by culturing for several days to several weeks. Subsequently, hybridomas that produce the antibody of interest can be screened and singly-cloned by carrying out a standard limiting dilution method. Alternatively, a Claudin 3-recognizing antibody can be prepared using the method described in International Patent Publication No. WO 03/104453.

An antibody of interest can be suitably screened and singly cloned by a screening method based on known antigen-antibody reaction. For example, preferred monoclonal antibodies of the present invention can bind to Claudin 3 expressed on the cell surface. Such monoclonal antibodies can be screened by fluorescence-activated cell sorting (FACS). FACS is a system that can be used to assess the binding of an antibody to the cell surface, by analyzing cells contacted with a fluorescent-labeled antibody using a laser beam, and measuring the fluorescence emitted by each cell.

To screen for hybridomas that produce monoclonal antibodies of the present invention by FACS, Claudin 3-expressing cells are prepared. Preferred cells for the screening are mammalian cells forced to express Claudin 3. By using untransformed mammalian host cells as the control, the activity of an antibody to bind to cell-surface Claudin 3 can be selectively detected. More specifically, hybridomas producing preferable monoclonal antibodies of the present invention can be obtained by selecting hybridomas that produce antibodies which do not bind to the untransformed host cells but bind to cells forced to express Claudin 3.

Alternatively, the activity of an antibody to bind to immobilized Claudin 3-expressing cells can be evaluated using the ELISA method. For example, Claudin 3-expressing cells are immobilized in the wells of an ELISA plate. A hybridoma culture supernatant is contacted with the immobilized cells in the wells, and the antibodies that bind to the immobilized cells are detected. If the monoclonal antibodies are derived from mice, the antibodies bound to the cells can be detected using anti-mouse immunoglobulin antibodies. Hybridomas selected by the screening, which produce the antibodies of interest having antigen-binding ability, can be cloned by the limiting dilution method or the like.

Alternatively, screening can be performed, for example, as follows to obtain monoclonal antibodies that recognize a conformational epitope of Claudin 3. Cells that express a chimeric molecule produced by linking one of the two extracellular loops of Claudin 3 with the other extracellular loop of a Claudin 3-like molecule are prepared. For example, human Claudin 1 can be used as the Claudin 3-like molecule. More specifically, cells forced to express the following chimeric molecules are produced.

| | 3/1 chimera | 1/3 chimera | Native |
|---|---|---|---|
| Loop 1 | Claudin 3 | Claudin 1 | Claudin 3 |
| Loop 2 | Claudin 1 | Claudin 3 | Claudin 3 |

Monoclonal antibodies that recognize a conformational epitope of Claudin 3 of the present invention can be obtained by contacting these forced expression cells with test antibodies, and selecting monoclonal antibodies having lower binding activity to both 3/1 chimera-expressing cells and 1/3 chimera-expressing cells, than to native-type Claudin 3-expressing cells. The preferred cells for the screening are mammalian cells. The reactivity of the monoclonal antibodies to these cells can be determined by ELISA or FACS using the cells as antigen.

The monoclonal antibody-producing hybridomas produced in this manner can be passaged and cultured in a standard medium. Alternatively, the hybridomas can be stored for a long period in liquid nitrogen.

The hybridomas can be cultured according to a standard method, and the monoclonal antibody of interest can be obtained from the culture supernatants. Alternatively, the hybridomas can be grown by administering them to a compatible mammal, and monoclonal antibodies can be obtained as its ascites. The former method is suitable for obtaining highly purified antibodies.

In the present invention, an antibody encoded by an antibody gene cloned from antibody-producing cells can be used. The cloned antibody gene can be incorporated into a suitable vector and then introduced into a host to express the antibody. Methods for isolating an antibody gene, introducing the gene into a vector, and transforming host cells have been established (see for example, Vandamme, A. M. et al., Eur. J. Biochem. (1990) 192, 767-775).

For example, a cDNA encoding the variable region (V region) of an anti-Claudin 3 antibody can be obtained from hybridoma cells producing the anti-Claudin 3 antibody. Usually, in order to accomplish this, first, total RNA is extracted from the hybridoma. For example, the following methods can be used as methods for extracting mRNA from cells:

the guanidine ultracentrifugation method (Chirgwin, J. M. et al., Biochemistry (1979) 18, 5294-5299); and
the AGPC method (Chomczynski, P. et al., Anal. Biochem. (1987) 162, 156-159).

The extracted mRNA can be purified using an mRNA purification kit (GE Healthcare Bio-Sciences) or the like. Alternatively, kits for directly extracting total mRNA from cells, such as the QuickPrep mRNA Purification Kit (GE Healthcare Bio-Sciences), are also commercially available. Total RNA can be obtained from the hybridoma by using such kits. A cDNA encoding the antibody V region can be synthesized from the obtained mRNA using reverse transcriptase. cDNA can be synthesized using the AMV Reverse Transcriptase First-strand cDNA Synthesis Kit (SEIKAGAKU CORPORATION) or the like. To synthesize and amplify cDNA, the SMART RACE cDNA Amplification Kit (Clontech) and the 5′-RACE method using PCR (Frohman, M. A. et al., Proc. Natl. Acad. Sci. USA (1988) 85, 8998-9002; Belyavsky, A. et al., Nucleic Acids Res. (1989) 17, 2919-2932) can be used. Furthermore, in the process of such cDNA synthesis, appropriate restriction enzyme sites, which will be described later, can be introduced into both ends of the cDNA.

The cDNA fragment of interest is purified from the obtained PCR product, and then ligated to a vector DNA. The recombinant vector is prepared in this manner and introduced into Escherichia coli or the like, and after colonies are selected, the desired recombinant vector can be prepared from the E. coli that formed the colonies. Whether or not the recombinant vector has the cDNA nucleotide sequence of interest can be confirmed by a known method, such as the dideoxynucleotide chain termination method.

To obtain a gene encoding a variable region, it is most convenient to use the 5′-RACE method which utilizes primers for amplifying the variable region gene. First, a 5′-RACE cDNA library is obtained by synthesizing cDNAs using RNAs extracted from hybridoma cells as template. To synthesize a 5′-RACE cDNA library, it is convenient to use commercially available kits such as the SMART RACE cDNA Amplification Kit.

The antibody genes are amplified by the PCR method, using the obtained 5′-RACE cDNA library as a template. Primers for amplification of mouse antibody genes can be designed based on known antibody gene sequences. The nucleotide sequences of these primers vary depending on the immunoglobulin subclass. Therefore, the subclasses are desirably determined in advance using a commercially available kit such as the IsoStrip Mouse Monoclonal Antibody Isotyping Kit (Roche Diagnostics).

More specifically, for example, when the objective is to obtain genes encoding mouse IgG, one may use primers that can amplify genes encoding γ1, γ2a, γ2b, and γ3 as the heavy chain and the κ chain and λ chain as the light chain. To amplify genes of the IgG variable region, generally, a primer that anneals to a portion corresponding to the constant region close to the variable region is used as the 3′-end primer. For the 5′-end primer, the primer included in a 5′-RACE cDNA library production kit can be used.

PCR products amplified in this manner can be used to reconstitute an immunoglobulin comprising a combination of heavy and light chains. Based on the binding activity of the reconstituted immunoglobulin to Claudin 3, one can screen for antibodies of interest.

For example, when the objective is to obtain an antibody against Claudin 3, preferably, the binding of the antibody to Claudin 3 is specific. One can screen for an antibody that binds to Claudin 3, for example, by the following steps:

(1) contacting an antibody comprising the V regions encoded by a cDNA obtained from a hybridoma with a Claudin 3-expressing cell;
(2) detecting binding between the Claudin 3-expressing cell and the antibody; and
(3) selecting the antibody that binds to the Claudin 3-expressing cell.

A method for detecting binding between an antibody and a Claudin 3-expressing cell is known. Specifically, the binding between an antibody and a Claudin 3-expressing cell can be detected by the aforementioned methods such as FACS. To evaluate the binding activity of an antibody, a fixed sample of a Claudin 3-expressing cell may also be used.

Alternatively, for an antibody screening method based on the binding activity, a phage vector-based panning method may be used. When the antibody genes are obtained as libraries of the heavy-chain and light-chain subclasses from polyclonal antibody-expressing cells, phage displaying methods are advantageous. Genes encoding variable regions of the heavy and light chains can be made into a single-chain Fv(scFv) gene by linking the genes via suitable linker sequences. Phages expressing an scFv on their surface can be obtained by inserting a gene encoding the scFv into a phagemid vector. DNA encoding an scFv having the binding activity of interest can be collected by contacting the phage with an antigen of interest, and then collecting antigen-bound phage. scFv having the binding activity of interest can be enriched by repeating this operation as necessary.

An antibody-encoding polynucleotide of the present invention may encode a full-length antibody or a portion of the antibody. “A portion of an antibody” refers to any portion of an antibody molecule. Hereinafter, the term “antibody fragment” may be used to refer to a portion of an antibody. A preferred antibody fragment of the present invention comprises the complementarity determination region (CDR) of an antibody. More preferably, an antibody fragment of the present invention comprises all of the three CDRs that constitute a variable region.

Once a cDNA encoding the V region of an anti-Claudin 3 antibody of interest is obtained, this cDNA is digested with restriction enzymes that recognize the restriction enzyme sites inserted to both ends of the cDNA. A preferred restriction enzyme recognizes and digests a nucleotide sequence that is less likely to appear in the nucleotide sequence constituting the antibody gene. Furthermore, to insert a single copy of the digested fragment into a vector in the correct direction, a restriction enzyme that provides sticky ends is preferred. A cDNA encoding the anti-Claudin 3 antibody V region, which has been digested as described above, is inserted into a suitable expression vector to obtain the antibody expression vector. In this step, a chimeric antibody can be obtained by fusing a gene encoding the antibody constant region (C region) with the above-mentioned gene encoding the V region in frame. Herein, “chimeric antibody” refers to an antibody whose constant and variable regions are derived from different origins. Therefore, in addition to interspecies chimeric antibodies such as mouse-human chimeric antibodies, human-human intraspecies chimeric antibodies are also included in the chimeric antibodies of the present invention. A chimeric antibody expression vector can also be constructed by inserting the aforementioned V-region gene into an expression vector into which a constant region gene has been introduced. More specifically, for example, the restriction enzyme recognition sequence for a restriction enzyme that digests the aforementioned V-region gene can be placed at the 5′ end of a DNA encoding a desired antibody constant region (C region) in an expression vector. The chimeric antibody expression vector is constructed by digesting the two vectors using the same combination of restriction enzymes, and fusing them in frame.

To produce an anti-Claudin 3 monoclonal antibody of the present invention, the antibody gene can be incorporated into an expression vector so that it is expressed under the regulation of an expression control region. The expression regulatory region for antibody expression includes, for example, an enhancer or a promoter. Then, by transforming suitable host cells with this expression vector, recombinant cells that carry the DNA expressing the anti-Claudin 3 antibody can be obtained.

To express an antibody gene, a DNA encoding the antibody heavy chain (H-chain) and a DNA encoding the antibody light chain (L-chain) can be incorporated separately into expression vectors. An antibody molecule comprising the H chain and L chain can be expressed by simultaneously transfecting (co-transfecting) the H-chain and L-chain-incorporated vectors into the same host cell. Alternatively, DNAs encoding the H chain and L chain can be incorporated into a single expression vector to transform a host cell with the vector (see International Patent Publication No. WO 94/11523).

Many combinations of hosts and expression vectors for isolating an antibody gene and then introducing the gene into an appropriate host to produce the antibody are known. Any of these expression systems can be applied to the present invention. When using eukaryotic cells as a host, animal cells, plant cells, and fungal cells can be used. More specifically, animal cells that may be used in the present invention are, for example, the following cells:

(1) mammalian cells such as CHO, COS, myeloma, baby hamster kidney (BHK), HeLa, and Vero cells;
(2) amphibian cells such as Xenopus oocytes; and
(3) insect cells such as sf9, sf21, Tn5.

In addition, as a plant cell system, an antibody gene expression system using cells derived from the Nicotiana genus such as Nicotiana tabacum is known. Callus-cultured cells can be used to transform plant cells.

Furthermore, the following cells can be used as fungal cells; yeasts: the Saccharomyces genus, for example, Saccharomyces cerevisiae, and the Pichia genus, for example, Pichia pastoris; and filamentous fungi: the Aspergillus genus, for example, Aspergillus niger.

Antibody gene expression systems that utilize prokaryotic cells are also known. For example, when using bacterial cells, E. coli cells, Bacillus subtilis cells, and such may be used in the present invention.

Expression vectors comprising the antibody genes of interest are introduced into these cells by transformation. By culturing the transformed cells in vitro, the desired antibodies can be obtained from the transformed cell culture.

In addition to the above host cells, transgenic animals can also be used to produce a recombinant antibody. That is, the antibody can be obtained from an animal into which the gene encoding the antibody of interest is introduced. For example, the antibody gene can be inserted in frame into a gene that encodes a protein produced inherently in milk to construct a fused gene. Goat β-casein or such can be used, for example, as the protein secreted in milk. A DNA fragment containing the fused gene inserted with the antibody gene is injected into a goat embryo, and then this embryo is introduced into a female goat. Desired antibodies can be obtained as a protein fused with the milk protein from milk produced by the transgenic goat born from the goat that received the embryo (or progeny thereof). To increase the volume of milk containing the desired antibody produced by the transgenic goat, hormones can be used on the transgenic goat as necessary (Ebert, K. M. et al., Bio/Technology (1994) 12, 699-702).

Animal-derived antibody C regions can be used for the C regions of a recombinant antibody of the present invention. For example, Cγ1, Cγ₂a, Cγ₂b, Cγ₃, Cμ, Cδ, Cα1, Cα2, and Cε can be used for the mouse antibody H-chain C-region, and Cκ and Cλ can be used for the L-chain C-region. In addition to mouse antibodies, antibodies of animals such as rats, rabbits, goat, sheep, camels, and monkeys can be used as animal antibodies. Their sequences are known. Furthermore, the C region can be modified to improve the stability of the antibodies or their production.

In the present invention, when administering antibodies to humans, genetically recombinant antibodies that have been artificially modified for the purpose of reducing xenoantigenicity against humans, or the like can be used. Examples of the genetically recombinant antibodies include chimeric antibodies and humanized antibodies. These modified antibodies can be produced using known methods.

A chimeric antibody is an antibody whose variable regions and constant regions are of different origins. For example, an antibody comprising the heavy-chain and light-chain variable regions of a mouse antibody and the heavy-chain and light-chain constant regions of a human antibody is a mouse-human interspecies chimeric antibody. A recombinant vector expressing a chimeric antibody can be produced by ligating a DNA encoding a mouse antibody variable region to a DNA encoding a human antibody constant region, and then inserting it into an expression vector. The recombinant cells that have been transformed with the vector are cultured, and the incorporated DNA is expressed to obtain the chimeric antibody produced in the culture. Human C regions are used for the C regions of chimeric antibodies and humanized antibodies.

For example, Cγ1, Cγ2, Cγ3, Cγ4, Cμ, Cδ, Cα1, Cα2, and Cε can be used as an H-chain C region. Cκ and Cλ can be used as an L-chain C region. The amino acid sequences of these C regions and the nucleotide sequences encoding them are known. Furthermore, the human antibody C region can be modified to improve the stability of an antibody or its production.

Generally, a chimeric antibody consists of the V region of an antibody derived from a non-human animal, and a C region derived from a human antibody. On the other hand, a humanized antibody consists of the complementarity determining region (CDR) of an antibody derived from a non-human animal, and the framework region (FR) and C region derived from a human antibody. Since the antigenicity of a humanized antibody in human body is reduced, a humanized antibody is useful as an active ingredient for therapeutic agents of the present invention.

For example, mouse-human chimeric antibodies obtained by linking the variable regions of an anti-Claudin 3 monoclonal antibody produced based on the present invention with amino acid sequences constituting the human constant regions are preferred as monoclonal antibodies of the present invention. More specifically, the present invention provides mouse-human chimeric monoclonal antibodies comprising an H-chain variable region and an L-chain variable region comprising the amino acid sequences of any of (1) to (6):

### (1) CDN04

H chain: the amino acid sequence of SEQ ID NO: 18
L chain: the amino acid sequence of SEQ ID NO: 30

### (2) CDN16

H chain: the amino acid sequence of SEQ ID NO: 42
L chain: the amino acid sequence of SEQ ID NO: 52

### (3) CDN27

H chain: the amino acid sequence of SEQ ID NO: 62
L chain: the amino acid sequence of SEQ ID NO: 72

### (4) CDN28

H chain: the amino acid sequence of SEQ ID NO: 82
L chain: the amino acid sequence of SEQ ID NO: 92

### (5) CDN35

H chain: the amino acid sequence of SEQ ID NO: 102
L chain: the amino acid sequence of SEQ ID NO: 112

### (6) CDN38

H chain: the amino acid sequence of SEQ ID NO: 165
L chain: the amino acid sequence of SEQ ID NO: 177

Furthermore, as an example of such mouse-human chimeric antibodies, the present invention provides a mouse-human chimeric antibody comprising an H chain comprising the amino acid sequence of SEQ ID NO: 175 and an L chain comprising the amino acid sequence of SEQ ID NO: 187, which is obtained by linking the CDN38 variable regions with the human constant regions.

The antibody variable region generally comprises three complementarity-determining regions (CDRs) separated by four framework regions (FRs). CDR is a region that substantially determines the binding specificity of an antibody. The amino acid sequences of CDRs are highly diverse. On the other hand, the FR-constituting amino acid sequences are often highly homologous even among antibodies with different binding specificities. Therefore, generally, the binding specificity of a certain antibody can be transferred to another antibody by CDR grafting.

A humanized antibody is also called a reshaped human antibody. Specifically, humanized antibodies prepared by grafting the CDR of a non-human animal antibody such as a mouse antibody to a human antibody and such are known. Common genetic engineering technologies for obtaining humanized antibodies are also known.

Specifically, for example, overlap extension PCR is known as a method for grafting a mouse antibody CDR to a human FR. In overlap extension PCR, a nucleotide sequence encoding a mouse antibody CDR to be grafted is added to the primers for synthesizing a human antibody FR. Primers are prepared for each of the four FRs. It is generally considered that when grafting a mouse CDR to a human FR, selecting a human FR that is highly homologous to a mouse FR is advantageous for maintaining the CDR function. That is, it is generally preferable to use a human FR comprising an amino acid sequence highly homologous to the amino acid sequence of the FR adjacent to the mouse CDR to be grafted.

Nucleotide sequences to be ligated are designed so that they will be connected to each other in frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, overlapping CDR regions of the products synthesized using a human antibody gene as the template are annealed for complementary strand synthesis reaction. By this reaction, human FRs are ligated through the mouse CDR sequences.

The full length of the V-region gene, in which three CDRs and four FRs are ultimately ligated, is amplified using primers that anneal to its 5′ and 3′ ends and which have suitable restriction enzyme recognition sequences. A vector for human antibody expression can be produced by inserting the DNA obtained as described above and a DNA that encodes a human antibody C region into an expression vector so that they will ligate in frame. After transfecting this integration vector into a host to establish recombinant cells, the recombinant cells are cultured, and the DNA encoding the humanized antibody is expressed to produce the humanized antibody in the cell culture (see, European Patent Publication No. EP 239,400, and International Patent Publication No. WO 96/02576).

By qualitatively or quantitatively measuring and evaluating the antigen-binding activity of the humanized antibody produced as described above, one can suitably select human antibody FRs that allow CDRs to form a favorable antigen-binding site when ligated through the CDRs. As necessary, amino acid residues in an FR may be substituted so that the CDRs of a reshaped human antibody form an appropriate antigen-binding site. For example, amino acid sequence mutations can be introduced into FRs by applying the PCR method used for fusing a mouse CDR with a human FR. More specifically, partial nucleotide sequence mutations can be introduced into primers that anneal to the FR sequence. Nucleotide sequence mutations are introduced into the FRs synthesized using such primers. Mutant FR sequences having the desired characteristics can be selected by measuring and evaluating the activity of the amino acid-substituted mutant antibody to bind to the antigen by the above-mentioned method (Sato, K. et al., Cancer Res. 1993, 53, 851-856).

Alternatively, a desired human antibody can be obtained by DNA immunization using a transgenic animal that comprises the entire repertoire of human antibody genes (see International Patent Publication Nos. WO 93/12227, WO 92/03918, WO 94/02602, WO 94/25585, WO 96/34096, and WO 96/33735) as an animal for immunization. Furthermore, technologies to obtain human antibodies by panning a human antibody library are also known. For example, the V region of a human antibody is expressed as a single chain antibody (scFv) on the phage surface using a phage display method, and phages that bind to the antigen can be selected. By analyzing the genes of selected phages, the DNA sequences encoding the V regions of human antibodies that bind to the antigen can be determined. After determining the DNA sequences of scFvs that bind to the antigen, the V region sequence is fused in frame with the desired human antibody C region sequence, and this is inserted into a suitable expression vector to produce an expression vector. This expression vector can be introduced into suitable expression cells such as those described above, and the human antibody-encoding gene can be expressed to obtain the human antibodies. Such methods are well known (International Patent Publication Nos. WO 92/01047, WO 92/20791, WO 93/06213, WO 93/11236, WO 93/19172, WO 95/01438, and WO 95/15388).

The monoclonal antibodies of the present invention are not limited to bivalent antibodies represented by IgG, but include monovalent antibodies and multivalent antibodies represented by IgM, as long as it binds to the Claudin 3 protein. The multivalent antibody of the present invention includes a multivalent antibody that has the same antigen binding sites, and a multivalent antibody that has partially or completely different antigen binding sites. The monoclonal antibody of the present invention is not limited to the whole antibody molecule, but includes minibodies and modified products thereof, as long as they bind to the Claudin 3 protein.

A minibody contains an antibody fragment lacking a portion of a whole antibody (for example, whole IgG). As long as it has the ability to bind the Claudin 3 antigen, partial deletions of an antibody molecule are permissible. Antibody fragments of the present invention preferably contain a heavy-chain variable region (VH) and/or a light-chain variable region (VL). The amino acid sequence of VH or VL may have substitutions, deletions, additions, and/or insertions. Furthermore, as long as it has the ability to bind the Claudin 3 antigen, VH and/or VL can be partially deleted. The variable region may be chimerized or humanized. Specific examples of the antibody fragments include Fab, Fab′, F(ab′)2, and Fv. Specific examples of minibodies include Fab, Fab′, F(ab′)2, Fv, scFv (single chain Fv), diabody, and sc(Fv)2 (single chain (Fv)2). Multimers of these antibodies (for example, dimers, trimers, tetramers, and polymers) are also included in the minibodies of the present invention.

Fragments of antibodies can be obtained by treating an antibody with an enzyme to produce antibody fragments. Known enzymes that produce antibody fragments are, for example, papain, pepsin, and plasmin. Alternatively, genes encoding these antibody fragments can be constructed, introduced into expression vectors, and then expressed in appropriate host cells (see, for example, Co, M. S. et al., J. Immunol. (1994) 152, 2968-2976; Better, M. and Horwitz, A. H., Methods in Enzymology (1989) 178, 476-496; Plueckthun, A. and Skerra, A., Methods in Enzymology (1989) 178, 476-496; Lamoyi, E., Methods in Enzymology (1989) 121, 652-663; Rousseaux, J. et al., Methods in Enzymology (1989) 121, 663-669; and Bird, R. E. et al., TIBTECH (1991) 9, 132-137).

Digestive enzymes cleave specific sites of an antibody fragment, and yield antibody fragments with the following specific structures. When genetic engineering technologies are used on such enzymatically obtained antibody fragments, any portion of the antibody can be deleted.

Papain digestion: F(ab)2 or Fab
Pepsin digestion: F(ab′)2 or Fab′
Plasmin digestion: Facb

Therefore, minibodies of the present invention may be antibody fragments lacking any region, as long as they have binding affinity to Claudin 3. Furthermore, according to the present invention, the antibodies desirably maintain their effector activity, particularly in the treatment of cell proliferative diseases such as cancer. More specifically, preferred minibodies of the present invention have both binding affinity to Claudin 3 and effector function. The antibody effector function includes ADCC activity and CDC activity. Particularly preferably, therapeutic antibodies of the present invention have ADCC activity and/or CDC activity as effector function.

A diabody refers to a bivalent antibody fragment constructed by gene fusion (Hollinger P. et al., Proc. Natl. Acad. Sci. USA 90: 6444-6448 (1993); EP 404,097; WO 93/11161; and such). A diabody is a dimer composed of two polypeptide chains. Generally, in each polypeptide chain constituting the dimer, VL and VH are linked by a linker within the same chain. The linker in a diabody is generally short enough to prevent binding between VL and VH. Specifically, the amino acid residues constituting the linker are, for example, five residues or so. Therefore, VL and VH that are encoded by the same polypeptide chain cannot form a single-chain variable region fragment, and form a dimer with another single chain variable region fragment. As a result, diabodies have two antigen binding sites.

scFv can be obtained by ligating the H-chain V region and L-chain V region of an antibody. In scFv, the H-chain V region and L-chain V region are ligated via a linker, preferably a peptide linker (Huston, J. S. et al., Proc. Natl. Acad. Sci. U.S.A., 1988, 85, 5879-5883). The H-chain V region and L-chain V region of scFv may be derived from any of the antibodies described herein. The peptide linker for ligating the V regions is not particularly limited. For example, any single-chain peptide consisting of 3 to 25 residues or so can be used as the linker. More specifically, for example, peptide linkers described below or such can be used.

PCR methods such as those described above can be used for ligating the V regions. For ligation of the V regions by PCR methods, first, a whole DNA or a DNA encoding a desired partial amino acid sequence selected from the following DNAs can be used as a template:

a DNA sequence encoding the H chain or the H-chain V region of the above-mentioned antibody; and
a DNA sequence encoding the L chain or the L-chain V region of the above-mentioned antibody.

DNAs encoding the H-chain and L-chain V regions are individually amplified by PCR methods using a pair of primers that have sequences corresponding to the sequences of both ends of the DNA to be amplified. Then, a DNA encoding the peptide linker portion is prepared. The DNA encoding the peptide linker can also be synthesized using PCR. To the 5′ end of the primers used, nucleotide sequences that can be ligated to each of the individually synthesized V-region amplification products are added. Then, PCR reaction is carried out using the “H-chain V region DNA”, “peptide linker DNA”, and “L-chain V region DNA”, and the primers for assembly PCR.

The primers for assembly PCR consist of the combination of a primer that anneals to the 5′ end of the “H-chain V region DNA” and a primer that anneals to the 3′ end of the “L-chain V region DNA”. That is, the primers for assembly PCR are a primer set that can amplify a DNA encoding the full-length sequence of scFv to be synthesized. On the other hand, nucleotide sequences that can be ligated to each V-region DNA are added to the “peptide linker DNA”. Thus, these DNAs are ligated, and the full-length scFv is ultimately produced as an amplification product using the primers for assembly PCR. Once the scFv-encoding DNA is constructed, expression vectors containing the DNA, and recombinant cells transformed by these expression vectors can be obtained according to conventional methods. Furthermore, the scFvs can be obtained by culturing the resulting recombinant cells and expressing the scFv-encoding DNA.

sc(Fv)2 is a minibody prepared by ligating two VHs and two VLs with linkers or such to form a single chain (Hudson et al., J. Immunol. Methods 1999; 231: 177-189). sc(Fv)2 can be produced, for example, by joining scFvs with a linker.

Moreover, antibodies in which two VHs and two VLs are arranged in the order of VH, VL, VH, and VL ([VH]-linker-[VL]-linker-[VH]-linker-[VL]), starting from the N-terminal side of a single chain polypeptide, are preferred.

The order of the two VHs and the two VLs is not particularly limited to the above-mentioned arrangement, and they may be placed in any order. Examples include the following arrangements:

[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

Any arbitrary peptide linker can be introduced by genetic engineering, and synthetic linkers (see, for example, those disclosed in Protein Engineering, 9(3), 299-305, 1996) or such can be used as linkers for linking the antibody variable regions. In the present invention, peptide linkers are preferable. The length of the peptide linkers is not particularly limited, and can be suitably selected by those skilled in the art according to the purpose. The length of amino acid residues composing a peptide linker is generally 1 to 100 amino acids, preferably 3 to 50 amino acids, more preferably 5 to 30 amino acids, and particularly preferably 12 to 18 amino acids (for example, 15 amino acids).

Any amino acid sequences composing peptide linkers can be used, as long as they do not inhibit the binding activity of scFv. Examples of the amino acid sequences used in peptide linkers include:

Ser
Gly-Ser
Gly-Gly-Ser
Ser-Gly-Gly
Gly-Gly-Gly-Ser (SEQ ID NO: 149)
Ser-Gly-Gly-Gly (SEQ ID NO: 150)
Gly-Gly-Gly-Gly-Ser (SEQ ID NO: 151)
Ser-Gly-Gly-Gly-Gly (SEQ ID NO: 152)
Gly-Gly-Gly-Gly-Gly-Ser (SEQ ID NO: 153)
Ser-Gly-Gly-Gly-Gly-Gly (SEQ ID NO: 154)
Gly-Gly-Gly-Gly-Gly-Gly-Ser (SEQ ID NO: 155)
Ser-Gly-Gly-Gly-Gly-Gly-Gly (SEQ ID NO: 156)
(Gly-Gly-Gly-Gly-Ser (SEQ ID NO: 157))n
(Ser-Gly-Gly-Gly-Gly (SEQ ID NO: 158))n

in which n is an integer of 1 or larger.

The amino acid sequences of the peptide linkers can be selected appropriately by those skilled in the art according to the purpose. For example, n, which determines the length of the peptide linkers, is generally 1 to 5, preferably 1 to 3, more preferably 1 or 2.

Therefore, a particularly preferred embodiment of sc(Fv)2 in the present invention is, for example, the following sc(Fv)2:

[VH]-peptide linker (15 amino acids)-[VL]-peptide linker (15 amino acids)-[VH]-peptide linker (15 amino acids)-[VL]

Alternatively, synthetic chemical linkers (chemical crosslinking agents) can be used to link the V regions. Crosslinking agents routinely used to crosslink peptide compounds and such can be used in the present invention. For example, the following chemical crosslinking agents are known. These crosslinking agents are commercially available:
N-hydroxy succinimide (NHS);
disuccinimidyl suberate (DSS);
bis(sulfosuccinimidyl) suberate (BS3);
dithiobis(succinimidyl propionate) (DSP);
dithiobis(sulfosuccinimidyl propionate) (DTSSP);
ethylene glycol bis(succinimidyl succinate) (EGS);
ethylene glycol bis(sulfosuccinimidyl succinate) (sulfo-EGS);
disuccinimidyl tartrate (DST);
disulfosuccinimidyl tartrate (sulfo-DST);
bis[2-(succinimidoxycarbonyloxy)ethyl]sulfone (BSOCOES); and
bis[2-(sulfosuccinimidoxycarbonyloxy)ethyl]sulfone (sulfo-BSOCOES).

Usually, three linkers are required to link four antibody variable regions. The multiple linkers to be used may all be of the same type or different types. In the present invention, a preferred minibody is a diabody or an sc(Fv)2. Such minibody can be obtained by treating an antibody with an enzyme, such as papain or pepsin, to generate antibody fragments, or by constructing DNAs that encode these antibody fragments, introducing them into expression vectors, and then expressing them in appropriate host cells (see, for example, Co, M. S. et al., J. Immunol. (1994) 152, 2968-2976; Better, M. and Horwitz, A. H., Methods Enzymol. (1989) 178, 476-496; Pluckthun, A. and Skerra, A., Methods Enzymol. (1989) 178, 497-515; Lamoyi, E., Methods Enzymol. (1986) 121, 652-663; Rousseaux, J. et al., Methods Enzymol. (1986) 121, 663-669; and Bird, R. E. and Walker, B. W., Trends Biotechnol. (1991) 9, 132-137).

Monoclonal antibodies of the present invention include any antibody that recognizes and binds to Claudin 3. For example, preferred antibodies include the antibodies of (1) to (61) shown below. These antibodies may be full-length antibodies, minibodies, animal antibodies, chimeric antibodies, humanized antibodies, or human antibodies.

(1) an antibody comprising an H chain having the amino acid sequence of SEQ ID NO: 12 as CDR1, the amino acid sequence of SEQ ID NO: 14 as CDR2, and the amino acid sequence of SEQ ID NO: 16 as CDR3;
(2) an antibody comprising the H chain of (1), wherein the H chain has the amino acid sequence of positions 139 to 462 in the amino acid sequence of SEQ ID NO: 20 as CH;
(3) an antibody comprising the H chain of (1), wherein the H chain has the amino acid sequence of SEQ ID NO: 22 as CH;
(4) an antibody comprising an L chain having the amino acid sequence of SEQ ID NO: 24 as CDR1, the amino acid sequence of SEQ ID NO: 26 as CDR2, and the amino acid sequence of SEQ ID NO: 28 as CDR3;
(5) an antibody comprising the L chain of (4), wherein the L chain has the amino acid sequence of positions 135 to 240 in the amino acid sequence of SEQ ID NO: 32 as CL;
(6) an antibody comprising the L chain of (4), wherein the L chain has the amino acid sequence of SEQ ID NO: 34 as CL;
(7) an antibody comprising the H chain of (1) and the L chain of (4);
(8) an antibody comprising the H chain of (2) and the L chain of (5);
(9) an antibody comprising the H chain of (3) and the L chain of (6);
(10) an antibody having one or more amino acid substitutions, deletions, additions, and/or insertions in the antibody of any of (1) to (9), which has equivalent activity as the antibody of any of (1) to (9);
(11) an antibody comprising an H chain having the amino acid sequence of SEQ ID NO: 36 as CDR1, the amino acid sequence of SEQ ID NO: 38 as CDR2, and the amino acid sequence of SEQ ID NO: 40 as CDR3;
(12) an antibody comprising the H chain of (11), wherein the H chain has the amino acid sequence of positions 140 to 476 in the amino acid sequence of SEQ ID NO: 44 as CH;
(13) an antibody comprising the H chain of (11), wherein the H chain has the amino acid sequence of SEQ ID NO: 22 as CH;
(14) an antibody comprising an L chain having the amino acid sequence of SEQ ID NO: 46 as CDR1, the amino acid sequence of SEQ ID NO: 48 as CDR2, and the amino acid sequence of SEQ ID NO: 50 as CDR3;
(15) an antibody comprising the L chain of (14), wherein the L chain has the amino acid sequence of positions 133 to 238 in the amino acid sequence of SEQ ID NO: 54 as CL;
(16) an antibody comprising the L chain of (14), wherein the L chain has the amino acid sequence of SEQ ID NO: 34 as CL;
(17) an antibody comprising the H chain of (11) and the L chain of (14);
(18) an antibody comprising the H chain of (12) and the L chain of (15);
(19) an antibody comprising the H chain of (13) and the L chain of (16);
(20) an antibody having one or more amino acid substitutions, deletions, additions, and/or insertions in the antibody of any of (11) to (19), which has equivalent activity as the antibody of any of (11) to (19);
(21) an antibody comprising an H chain having the amino acid sequence of SEQ ID NO: 56 as CDR1, the amino acid sequence of SEQ ID NO: 58 as CDR2, and the amino acid sequence of SEQ ID NO: 60 as CDR3;
(22) an antibody comprising the H chain of (21), wherein the H chain has the amino acid sequence of positions 137 to 471 in the amino acid sequence of SEQ ID NO: 64 as CH;
(23) an antibody comprising the H chain of (21), wherein the H chain has the amino acid sequence of SEQ ID NO: 22 as CH;
(24) an antibody comprising an L chain having the amino acid sequence of SEQ ID NO: 66 as CDR1, the amino acid sequence of SEQ ID NO: 68 as CDR2, and the amino acid sequence of SEQ ID NO: 70 as CDR3;
(25) an antibody comprising the L chain of (24), wherein the L chain has the amino acid sequence of positions 133 to 238 in the amino acid sequence of SEQ ID NO: 74 as CL;
(26) an antibody comprising the L chain of (24), wherein the L chain has the amino acid sequence of SEQ ID NO: 34 as CL;
(27) an antibody comprising the H chain of (21) and the L chain of (24);
(28) an antibody comprising the H chain of (22) and the L chain of (25);
(29) an antibody comprising the H chain of (23) and the L chain of (26);
(30) an antibody having one or more amino acid substitutions, deletions, additions, and/or insertions in the antibody of any of (21) to (29), which has equivalent activity as the antibody of any of (21) to (29);
(31) an antibody comprising an H chain having the amino acid sequence of SEQ ID NO: 76 as CDR1, the amino acid sequence of SEQ ID NO: 78 as CDR2, and the amino acid sequence of SEQ ID NO: 80 as CDR3;
(32) an antibody comprising the H chain of (31), wherein the H chain has the amino acid sequence of positions 140 to 463 in the amino acid sequence of SEQ ID NO: 84 as CH;
(33) an antibody comprising the H chain of (31), wherein the H chain has the amino acid sequence of SEQ ID NO: 22 as CH;
(34) an antibody comprising an L chain having the amino acid sequence of SEQ ID NO: 86 as CDR1, the amino acid sequence of SEQ ID NO: 88 as CDR2, and the amino acid sequence of SEQ ID NO: 90 as CDR3;
(35) an antibody comprising the L chain of (34), wherein the L chain has the amino acid sequence of positions 133 to 238 in the amino acid sequence of SEQ ID NO: 94 as CL;
(36) an antibody comprising the L chain of (34), wherein the L chain has the amino acid sequence of SEQ ID NO: 34 as CL;
(37) an antibody comprising the H chain of (31) and the L chain of (34);
(38) an antibody comprising the H chain of (32) and the L chain of (35);
(39) an antibody comprising the H chain of (33) and the L chain of (36);
(40) an antibody having one or more amino acid substitutions, deletions, additions, and/or insertions in the antibody of any of (31) to (39), which has equivalent activity as the antibody of any of (31) to (39);
(41) an antibody comprising an H chain having the amino acid sequence of SEQ ID NO: 96 as CDR1, the amino acid sequence of SEQ ID NO: 98 as CDR2, and the amino acid sequence of SEQ ID NO: 100 as CDR3;
(42) an antibody comprising the H chain of (41), wherein the H chain has the amino acid sequence of positions 140 to 474 in the amino acid sequence of SEQ ID NO: 104 as CH;
(43) an antibody comprising the H chain of (41), wherein the H chain has the amino acid sequence of SEQ ID NO: 22 as CH;
(44) an antibody comprising an L chain having the amino acid sequence of SEQ ID NO: 106 as CDR1, the amino acid sequence of SEQ ID NO: 108 as CDR2, and the amino acid sequence of SEQ ID NO: 110 as CDR3;
(45) an antibody comprising the L chain of (44), wherein the L chain has the amino acid sequence of positions 133 to 238 in the amino acid sequence of SEQ ID NO: 114 as CL;
(46) an antibody comprising the L chain of (44), wherein the L chain has the amino acid sequence of SEQ ID NO: 34 as CL;
(47) an antibody comprising the H chain of (41) and the L chain of (44);
(48) an antibody comprising the H chain of (42) and the L chain of (45);
(49) an antibody comprising the H chain of (43) and the L chain of (46);
(50) an antibody having one or more amino acid substitutions, deletions, additions, and/or insertions in the antibody of any of (41) to (49), which has equivalent activity as the antibody of any of (41) to (49);
(51) an antibody comprising an H chain having the amino acid sequence of SEQ ID NO: 167 as CDR1, the amino acid sequence of SEQ ID NO: 169 as CDR2, and the amino acid sequence of SEQ ID NO: 171 as CDR3;
(52) an antibody comprising the H chain of (51), wherein the H chain has the amino acid sequence of positions 118 to 447 in the amino acid sequence of SEQ ID NO: 173 as CH;
(53) an antibody comprising the H chain of (51), wherein the H chain has the amino acid sequence of SEQ ID NO: 22 as CH;
(54) an antibody comprising an L chain having the amino acid sequence of SEQ ID NO: 179 as CDR1, the amino acid sequence of SEQ ID NO: 181 as CDR2, and the amino acid sequence of SEQ ID NO: 183 as CDR3;
(55) an antibody comprising the L chain of (54), wherein the L chain has the amino acid sequence of positions 113 to 218 in the amino acid sequence of SEQ ID NO: 185 as CL;
(56) an antibody comprising the L chain of (54), wherein the L chain has the amino acid sequence of SEQ ID NO: 34 as CL;
(57) an antibody comprising the H chain of (51) and the L chain of (54);
(58) an antibody comprising the H chain of (52) and the L chain of (55);
(59) an antibody comprising the H chain of (53) and the L chain of (56);
(60) an antibody having one or more amino acid substitutions, deletions, additions, and/or insertions in the antibody of any of (51) to (59), which has equivalent activity as the antibody of any of (51) to (59);
(61) an antibody that binds to the same epitope as the Claudin 3 protein epitope bound by the antibody of any of (1) to (60).

In the present invention, preferred monoclonal antibodies comprise the amino acid sequences constituting CDR1, CDR2, and CDR3 of the heavy chains and light chains derived from any of CDN04, CDN16, CDN27, CDN28, CDN35, and CDN38 as the CDR amino acid sequences. The CDR amino acid sequences of these monoclonal antibodies are shown below. The monoclonal antibodies comprising in their variable regions, CDRs comprising the amino acid sequences shown in the sequence ID numbers indicated below, are preferred as the monoclonal antibodies of the present invention. For example, the V region sequences and full-length amino acid sequences of the monoclonal antibodies are shown in the sequence ID numbers indicated below. The monoclonal antibodies comprising these amino acid sequences in the V regions can be indicated as preferred monoclonal antibodies of the present invention.

| | | Heavy chain | Light chain |
|---|---|---|---|
| CDN04 | CDR1 | SEQ ID NO: 12 | SEQ ID NO: 24 |
| | CDR2 | SEQ ID NO: 14 | SEQ ID NO: 26 |
| | CDR3 | SEQ ID NO: 16 | SEQ ID NO: 28 |
| [CDN04 | V region | SEQ ID NO: 18 | SEQ ID NO: 30] |
| [CDN04 | C region | positions 139-462 of | positions 135-240 of |
| | | SEQ ID NO: 20 | SEQ ID NO: 32] |
| [CDN04 | full length | SEQ ID NO: 20 | SEQ ID NO: 32] |
| CDN16 | CDR1 | SEQ ID NO: 36 | SEQ ID NO: 46 |
| | CDR2 | SEQ ID NO: 38 | SEQ ID NO: 48 |
| | CDR3 | SEQ ID NO: 40 | SEQ ID NO: 50 |
| [CDN16 | V region | SEQ ID NO: 42 | SEQ ID NO: 52] |
| [CDN16 | C region | positions 140-476 of | positions 133-238 of |
| | | SEQ ID NO: 44 | SEQ ID NO: 54] |
| [CDN16 | full length | SEQ ID NO: 44 | SEQ ID NO: 54] |
| CDN27 | CDR1 | SEQ ID NO: 56 | SEQ ID NO: 66 |
| | CDR2 | SEQ ID NO: 58 | SEQ ID NO: 68 |
| | CDR3 | SEQ ID NO: 60 | SEQ ID NO: 70 |
| [CDN27 | V region | SEQ ID NO: 62 | SEQ ID NO: 72] |
| [CDN27 | C region | positions 137-471 of | positions 133-234 of |
| | | SEQ ID NO: 64 | SEQ ID NO: 74] |
| [CDN27 | full length | SEQ ID NO: 64 | SEQ ID NO: 74] |
| CDN28 | CDR1 | SEQ ID NO: 76 | SEQ ID NO: 86 |
| | CDR2 | SEQ ID NO: 78 | SEQ ID NO: 88 |
| | CDR3 | SEQ ID NO: 80 | SEQ ID NO: 90 |
| [CDN28 | V region | SEQ ID NO: 82 | SEQ ID NO: 92] |
| [CDN28 | C region | positions 140-463 of | positions 133-238 of |
| | | SEQ ID NO: 84 | SEQ ID NO: 94] |
| [CDN28 | full length | SEQ ID NO: 84 | SEQ ID NO: 94] |
| CDN35 | CDR1 | SEQ ID NO: 96 | SEQ ID NO: 106 |
| | CDR2 | SEQ ID NO: 98 | SEQ ID NO: 108 |
| | CDR3 | SEQ ID NO: 100 | SEQ ID NO: 110 |
| [CDN35 | V region | SEQ ID NO: 102 | SEQ ID NO: 112] |
| [CDN35 | C region | positions 140-474 of | positions 133-238 of |
| | | SEQ ID NO: 104 | SEQ ID NO: 114] |
| [CDN35 | full length | SEQ ID NO: 104 | SEQ ID NO: 114] |
| CDN38 | CDR1 | SEQ ID NO: 167 | SEQ ID NO: 179 |
| | CDR2 | SEQ ID NO: 169 | SEQ ID NO: 181 |
| | CDR3 | SEQ ID NO: 171 | SEQ ID NO: 183 |
| [CDN38 | V region | SEQ ID NO: 165 | SEQ ID NO: 177] |
| [CDN38 | C region | positions 118-447 of | positions 113-218 of |
| | | SEQ ID NO: 173 | SEQ ID NO: 185] |
| [CDN38 | full length | SEQ ID NO: 173 | SEQ ID NO: 185] |

The monoclonal antibodies of the present invention may comprise a constant region in addition to a variable region comprising the aforementioned CDRs. The full-length sequences of the monoclonal antibodies including the constant regions are as shown above. Furthermore, the following human-derived amino acid sequences can be shown as examples of the constant regions comprised in the monoclonal antibodies of the present invention:

SEQ ID NO: 21 (human IgG1 CH sequence), SEQ ID NO: 33 (human IgG1 CL kappa sequence),
SEQ ID NO: 22 (human IgG1 CH sequence), SEQ ID NO: 34 (human IgG1 CL kappa sequence)

Therefore, the monoclonal antibodies produced by linking the constant regions comprising the human-derived amino acid sequences shown by the above-mentioned sequence

ID numbers with the variable regions comprising the aforementioned CDRs 1, 2, and 3 are preferable monoclonal antibodies of the present invention. Examples of such monoclonal antibodies include the above-mentioned monoclonal antibodies of (3), (13), (23), (33), (43), and (53), and may include the above-mentioned light chains of (6), (16), (26), (36), (46), and (56), respectively, as the light chains.

A preferred embodiment of the above-mentioned antibody of (10), (20), (30), (40), (50), or (60) is an antibody in which the CDR has not been modified. For example, among the above-mentioned antibodies of (10), a preferred embodiment of “an antibody having one or more amino acid substitutions, deletions, additions, and/or insertions in the antibody of (1) and having an activity equivalent to that of the antibody of (1)” is “an antibody having an activity equivalent to that of the antibody of (1) and having one or more amino acid substitutions, deletions, additions, and/or insertions in the antibody of (1), and also comprising an H chain having the amino acid sequence of SEQ ID NO: 12 as CDR1, the amino acid sequence of SEQ ID NO: 14 as CDR2, and the amino acid sequence of SEQ ID NO: 16 as CDR3”. Preferred embodiments of other antibodies included in the above-mentioned antibody of (10), (20), (30), (40), (50), or (60) can be expressed in a similar manner.

A method of introducing mutations into polypeptides is one of the methods well known to those skilled in the art for preparing polypeptides that are functionally equivalent to a certain polypeptide. For example, those skilled in the art can prepare an antibody functionally equivalent to an antibody of the present invention by introducing appropriate mutations into the antibody using site-directed mutagenesis (Hashimoto-Gotoh, T. et al. (1995) Gene 152, 271-275; Zoller, M J, and Smith, M. (1983) Methods Enzymol. 100, 468-500; Kramer, W. et al. (1984) Nucleic Acids Res. 12, 9441-9456; Kramer W, and Fritz H J (1987) Methods. Enzymol. 154, 350-367; Kunkel, T A (1985) Proc. Natl. Acad. Sci. USA. 82, 488-492; Kunkel (1988) Methods Enzymol. 85, 2763-2766) and such. Amino acid mutations may also occur naturally. In this way, the antibodies of the present invention also comprise antibodies comprising amino acid sequences with one or more amino acid mutations in the amino acid sequences of the antibodies of the present invention, and which are functionally equivalent to the antibodies of the present invention.

The number of amino acids that are mutated in such mutants is generally considered to be 50 amino acids or less, preferably 30 amino acids or less, and more preferably 10 amino acids or less (for example, 5 amino acids or less).

It is desirable that the amino acid residues are mutated into amino acids in which the properties of the amino acid side chains are conserved. For example, the following categories have been established depending on the amino acid side chain properties:

hydrophobic amino acids (A, I, L, M, F, P, W, Y, and V);
hydrophilic amino acids (R, D, N, C, E, Q, G, H, K, S, and T);
amino acids having aliphatic side chains (G, A, V, L, I, and P);
amino acids having hydroxyl-containing side chains (S, T, and Y);
amino acids having sulfur-containing side chains (C and M);
amino acids having carboxylic acid- and amide-containing side chains (D, N, E, and Q);
amino acids having basic side chains (R, K, and H); and
amino acids having aromatic ring-containing side chains (H, F, Y, and W)
(amino acids are represented by one-letter codes in parentheses).

Polypeptides comprising a modified amino acid sequence, in which one or more amino acid residues in a certain amino acid sequence is deleted, added, and/or substituted with other amino acids, are known to retain their original biological activities (Mark, D. F. et al., Proc. Natl. Acad. Sci. USA (1984) 81, 5662-5666; Zoller, M. J. & Smith, M. Nucleic Acids Research (1982) 10, 6487-6500; Wang, A. et al., Science 224, 1431-1433; Dalbadie-McFarland, G. et al., Proc. Natl. Acad. Sci. USA (1982) 79, 6409-6413). That is, generally in an amino acid sequence constituting a certain polypeptide, the activity of the polypeptide is highly likely to be maintained when amino acids classified into the same group are mutually substituted. In the present invention, the above-mentioned substitution between amino acids within the same amino acid group is referred to as conservative substitution.

The present invention provides antibodies that bind to the same epitope as the monoclonal antibodies disclosed in the present application. More specifically, the present invention relates to antibodies that recognize the same epitope as the monoclonal antibodies of the present invention, and uses thereof. Such antibodies can be obtained, for example, by the following method.

Whether a test antibody binds to the same epitope as the epitope bound by a certain antibody; that is, whether a test antibody shares the epitope of a certain antibody can be confirmed by checking whether the two antibodies compete for the same epitope. In the present invention, competition between antibodies can be detected by FACS or cross-blocking assay. In FACS, first, a monoclonal antibody of the present invention is bound to Claudin 3-expressing cells, and the fluorescence signal is detected. Next, a candidate competitive antibody is reacted with the cells, then the monoclonal antibody of the present invention is reacted with the same cells, and this is analyzed similarly by FACS. Alternatively, a monoclonal antibody of the present invention and a test competitive antibody can be reacted with the same cells at the same time. If the pattern of FACS analysis of a monoclonal antibody of the present invention changes upon reaction with a competitive antibody, one can confirm that the competitive antibody recognizes the same epitope as the monoclonal antibody of the present invention.

Alternatively, for example, competitive ELISA assay is a preferred cross-blocking assay. Specifically, in a cross-blocking assay, Claudin 3 protein-expressing cells are immobilized onto the wells of a microtiter plate. After preincubation in the presence or absence of a candidate competitive antibody, a monoclonal antibody of the present invention is added. The amount of monoclonal antibody of the present invention that binds to the Claudin 3 protein-expressing cells in the wells inversely correlates with the binding ability of the candidate competitive antibody (test antibody) that competes for binding to the same epitope. That is, the greater the affinity the test antibody has for the same epitope, the lower the amount of the monoclonal antibody of the present invention bound to the wells onto which the Claudin 3 protein-expressing cells are immobilized. On the other hand, the greater the affinity the test antibody has for the same epitope, the greater the amount of the test antibody bound to the wells onto which the Claudin 3 protein-expressing cells are immobilized.

The amount of antibodies bound to the wells can be easily determined by labeling the antibodies in advance. For example, biotin-labeled antibodies can be detected using an avidin peroxidase conjugate and its suitable substrate. Cross-blocking assays that use the antibody labeled with an enzyme such as peroxidase are specifically called competitive ELISA assays. The antibodies can be labeled with other detectable or measurable substances. Specifically, radioactive labeling and fluorescent labeling are known.

Furthermore, when the test antibody has a constant region derived from a species different from that of the monoclonal antibody of the present invention, measurement can be done for either one of the antibodies bound to the wells using a labeled antibody that specifically recognizes the constant region derived from the species of the antibody to be detected. Alternatively, if the antibodies are derived from the same species but belong to different classes, the antibodies bound to the wells can be measured using antibodies that specifically distinguish individual classes.

If a candidate competing antibody can block binding of a monoclonal antibody of the present invention by at least 20%, preferably by at least 20% to 50%, and even more preferably, by at least 50%, as compared to the binding activity obtained in a control experiment performed in the absence of the candidate competing antibody, the candidate competing antibody is either an antibody that binds substantially to the same epitope or one that competes for binding to the same epitope as a monoclonal antibody of the present invention.

Antibodies that bind to the same epitope as the monoclonal antibodies include, for example, the above-mentioned antibody of (61).

As described above, the above-mentioned antibodies of (1) to (61) include not only monovalent antibodies but also multivalent antibodies. Multivalent antibodies of the present invention include multivalent antibodies whose antigen binding sites are all the same and multivalent antibodies whose antigen binding sites are partially or completely different.

Antibodies bound to various types of molecules such as polyethylene glycol (PEG) can also be used as modified antibodies. Moreover, chemotherapeutic agents, toxic peptides, or radioactive chemical substances can be bound to the antibodies. Such modified antibodies (hereinafter referred to as antibody conjugates) can be obtained by subjecting the obtained antibodies to chemical modification. Methods for modifying antibodies are already established in this field. Furthermore, as described below, such antibodies can also be obtained in the molecular form of a bispecific antibody designed using genetic engineering technologies to recognize not only Claudin 3 proteins, but also chemotherapeutic agents, toxic peptides, radioactive chemical compounds, or such. These antibodies are included in the “antibodies” of the present invention.

Chemotherapeutic agents that are bound to monoclonal antibodies of the present invention to drive the cytotoxic activity include the following:

azaribine, anastrozole, azacytidine, bleomycin, bortezomib,

bryostatin-1, busulfan, camptothecin, 10-hydroxycamptothecin, carmustine,

celebrex, chlorambucil, cisplatin, irinotecan, carboplatin, cladribine,

cyclophosphamide, cytarabine, dacarbazine, docetaxel, dactinomycin,

daunomycin glucuronide, daunorubicin, dexamethasone, diethylstilbestrol,

doxorubicin, doxorubicin glucuronide, epirubicin, ethinyl estradiol,

estramustine, etoposide, etoposide glucuronide, floxuridine, fludarabine,

flutamide, fluorouracil, fluoxymesterone, gemcitabine,

hydroxyprogesterone caproate, hydroxyurea, idarubicin, ifosfamide,

leucovorin, lomustine, mechlorethamine, medroxyprogesterone acetate,

megestrol acetate, melphalan, mercaptopurine, methotrexate, mitoxantrone,

mithramycin, mitomycin, mitotane, phenylbutyrate, prednisone, procarbazine,

paclitaxel, pentostatin, semustine streptozocin, tamoxifen, taxanes,

taxol, testosterone propionate, thalidomide, thioguanine,

thiotepa, teniposide, topotecan, uracil mustard, vinblastine,

vinorelbine, and vincristine.

In the present invention, preferred chemotherapeutic agents are low-molecular-weight chemotherapeutic agents. Low-molecular-weight chemotherapeutic agents are unlikely to interfere with antibody function even after binding to antibodies. In the present invention, low-molecular-weight chemotherapeutic agents usually have a molecular weight of 100 to 2000, preferably 200 to 1000. Examples of the chemotherapeutic agents demonstrated herein are all low-molecular-weight chemotherapeutic agents. The chemotherapeutic agents of the present invention include prodrugs that are converted to active chemotherapeutic agents in vivo. Prodrug activation may be enzymatic conversion or non-enzymatic conversion.

Furthermore, the antibodies can be modified using toxic peptides such as ricin, abrin, ribonuclease, onconase, DNase I, Staphylococcal enterotoxin-A, pokeweed antiviral protein, gelonin, diphtheria toxin, Pseudomonas exotoxin, Pseudomonas endotoxin, L-asparaginase, and PEG L-Asparaginase. In another embodiment, one or two or more of the low-molecular-weight chemotherapeutic agents and toxic peptides can be combined and used for antibody modification. The bonding between a monoclonal antibody and the above-mentioned low-molecular weight chemotherapeutic agent may be covalent bonding or non-covalent bonding. Methods for producing antibodies bound to these chemotherapeutic agents are known.

Furthermore, pharmacologically active proteins or peptide toxins can be bound to antibodies by gene recombination technologies. Specifically, for example, it is possible to construct a recombinant vector by fusing a DNA encoding the above-mentioned toxic peptide with a DNA encoding a monoclonal antibody of the present invention in frame, and inserting this into an expression vector. This vector is introduced into suitable host cells, the obtained transformed cells are cultured, and the incorporated DNA is expressed. Thus, an anti-Claudin 3 antibody bound to the toxic peptide can be obtained as a fusion protein. When obtaining an antibody as a fusion protein, the pharmacologically active protein or toxin is generally fused at the C terminus of the antibody. A peptide linker can be inserted between the antibody and the pharmacologically active protein or toxin.

Furthermore, the monoclonal antibody of the present invention may be a bispecific antibody. A bispecific antibody refers to an antibody that carries variable regions that recognize different epitopes within the same antibody molecule. The bispecific antibody may have antigen-binding sites that recognize different epitopes on a Claudin 3 molecule. Two molecules of such a bispecific antibody can bind to one molecule of Claudin 3. As a result, stronger cytotoxic action can be expected.

Alternatively, the bispecific antibody may be an antibody in which one antigen-binding site recognizes Claudin 3, and the other antigen-binding site recognizes a cytotoxic substance. Specifically, cytotoxic substances include chemotherapeutic agents, toxic peptides, and radioactive chemical substances. Such a bispecific antibody binds to Claudin 3-expressing cells, and at the same time, captures cytotoxic substances. This enables the cytotoxic substances to directly act on Claudin 3-expressing cells. Therefore, bispecific antibodies that recognize cytotoxic substances specifically injure tumor cells and suppress tumor cell proliferation.

Furthermore, in the present invention, bispecific antibodies that recognize antigens other than Claudin 3 may be combined. For example, it is possible to combine bispecific antibodies that recognize non-Claudin 3 antigens that are specifically expressed on the surface of target cancer cells like Claudin 3.

Methods for producing bispecific antibodies are known. For example, two types of antibodies recognizing different antigens may be linked to prepare a bispecific antibody. The antibodies to be linked may be half molecules each having an H chain or an L chain, or may be quarter molecules consisting of only an H chain. Alternatively, hybrid cells producing a bispecific antibody can be prepared by fusing hybridomas producing different monoclonal antibodies. Bispecific antibodies can also be prepared by genetic engineering technologies.

Antibody genes constructed as described above can be expressed and antibodies can be obtained by known methods. For mammalian cells, the antibody genes can be expressed by operatively placing the antibody gene just behind a commonly used effective promoter, and a polyA signal on the 3′ downstream side of the antibody gene. An example of such promoter/enhancer is human cytomegalovirus immediate early promoter/enhancer.

Other promoters/enhancers that can be used for antibody expression include viral promoters/enhancers, or mammalian cell-derived promoters/enhancers such as human elongation factor 1α (HEF1α). Specific examples of viruses whose promoters/enhancers may be used include retrovirus, polyoma virus, adenovirus, and simian virus 40 (SV40).

When an SV40 promoter/enhancer is used, the method of Mulligan et al. (Nature (1979) 277, 108) may be utilized. An HEF1α promoter/enhancer can be readily used for expressing a gene of interest by the method of Mizushima et al. (Nucleic Acids Res. (1990) 18, 5322).

In the case of E. coli, the antibody can be expressed by operatively placing the antibody gene with a signal sequence for secretion at downstream of a commonly used effective promoter. Examples of such promoter include the lacZ promoter and araB promoter. For the lacZ promoter, the method of Ward et al., (Nature (1989) 341, 544-546; FASEB J. (1992) 6, 2422-2427) may be used. Alternatively, the araB promoter can be used for expressing a gene of interest by the method of Better et al. (Science (1988) 240, 1041-1043).

The pelB signal sequence for secretion (Lei, S. P. et al., J. Bacteriol. (1987) 169, 4379) may be used for antibody production in the periplasm of E. coli. After isolation of the antibody produced in the periplasm, the antibody can be refolded by using a protein denaturant like guanidine hydrochloride or urea so that the antibody will have the desired binding activity.

The replication origin inserted into the expression vector includes, for example, those derived from SV40, polyoma virus, adenovirus, or bovine papilloma virus (BPV). In order to amplify the gene copy number in the host cell system, a selection marker can be inserted into the expression vector. Specifically, the following selection markers can be used:

the aminoglycoside transferase (APH) gene;
the thymidine kinase (TK) gene;
the E. coli xanthine guanine phosphoribosyltransferase (Ecogpt) gene;
the dihydrofolate reductase (dhfr) gene, etc.

Any expression system, for example, a eukaryotic cell system or a prokaryotic cell system can be used to produce monoclonal antibodies of the present invention. Examples of eukaryotic cells include animal cells such as established mammalian cell lines, insect cell lines, and filamentous fungus cells and yeast cells. Examples of prokaryotic cells include bacterial cells such as E. coli cells. Monoclonal antibodies of the present invention are preferably expressed in mammalian cells. For example, mammalian cells such as CHO, COS, myeloma, BHK, Vero, or HeLa cells can be used.

Then, the transformed cell is then cultured in vitro or in vivo to produce an antibody of interest. The cells are cultured according to known methods. For example, DMEM, MEM, RPMI 1640, or IMDM can be used as the culture medium. A serum such as fetal calf serum (FCS) can also be used as supplement.

Antibodies produced as described above can be purified by using a single or a suitable combination of known methods generally used for purifying proteins. Antibodies can be separated and purified by, for example, appropriately combining filtration, ultrafiltration, salt precipitation, dialysis, affinity chromatography using a protein A column, other chromatography, and such (Antibodies A Laboratory Manual. Ed Harlow, David Lane, Cold Spring Harbor Laboratory, 1988).

Known methods can be used to measure the antigen-binding activity of the antibodies (Antibodies A Laboratory Manual. Ed Harlow, David Lane, Cold Spring Harbor Laboratory, 1988). For example, an enzyme linked immunosorbent assay (ELISA), an enzyme immunoassay (EIA), a radioimmunoassay (RIA), or a fluoroimmunoassay can be used.

The monoclonal antibodies of the present invention may be antibodies with a modified sugar chain. It is known that the cytotoxic activity of an antibody can be increased by modifying its sugar chain. Known antibodies having modified sugar chains include the following:

antibodies with modified glycosylation (for example, WO 99/54342);
antibodies deficient in fucose attached to sugar chains (for example, WO 00/61739 and WO 02/31140);
antibodies having a sugar chain with bisecting GlcNAc (for example, WO 02/79255), etc.

The antibodies used in the present invention are preferably antibodies having cytotoxic activity.

In the present invention, the cytotoxic activity includes, for example, antibody-dependent cell-mediated cytotoxicity (ADCC) activity and complement-dependent cytotoxicity (CDC) activity. In the present invention, CDC activity refers to complement system-mediated cytotoxic activity. ADCC activity refers to the activity of injuring a target cell when a specific antibody attaches to its cell surface antigen. An Fcγ receptor-carrying cell (immune cell, or such) binds to the Fc portion of the antibody via the Fcγ receptor and the target cell is damaged.

A monoclonal antibody of the present invention can be tested to see whether it has ADCC activity or CDC activity using known methods (for example, Current Protocols in Immunology, Chapter 7. Immunologic studies in humans, Editor, John E. Coligan et al., John Wiley & Sons, Inc., (1993) and the like).

First, specifically, effector cells, complement solution, and target cells are prepared.

(1) Preparation of Effector Cells

Spleen is removed from a CBA/N mouse or the like, and spleen cells are isolated in RPMI1640 medium (manufactured by Invitrogen). After washing in the same medium containing 10% fetal bovine serum (FBS, manufactured by HyClone), the cell concentration is adjusted to 5×10⁶/mL to prepare the effector cells.

(2) Preparation of Complement Solution

Baby Rabbit Complement (manufactured by CEDARLANE) is diluted 10-fold in a culture medium (manufactured by Invitrogen) containing 10% FBS to prepare a complement solution.

(3) Preparation of Target Cells

The target cells can be radioactively labeled by incubating cells expressing the Claudin 3 protein with 0.2 mCi of sodium chromate-⁵¹Cr (manufactured by GE Healthcare Bio-Sciences) in a DMEM medium containing 10% FBS for one hour at 37° C. For Claudin 3 protein-expressing cells, one may use transformed cells with a Claudin 3 gene, ovarian cancer cells, prostate cancer cells, breast cancer cells, uterine cancer cells, liver cancer cells, lung cancer cells, pancreatic cancer cells, stomach cancer cells, bladder cancer cells, colon cancer cells, or such. After radioactive labeling, cells are washed three times in RPMI1640 medium with 10% FBS, and the target cells can be prepared by adjusting the cell concentration to 2×10⁵/mL.

ADCC activity or CDC activity can be measured by the method described below. In the case of ADCC activity measurement, the target cell and anti-Claudin 3 antibody (50 μL each) are added to a 96-well U-bottom plate (manufactured by Becton Dickinson), and reacted for 15 minutes on ice. Thereafter, 100 μL of effector cells are added and incubated in a carbon dioxide incubator for four hours. The final concentration of the antibody is adjusted to 0 or 10 μg/mL. After culturing, 100 μL of the supernatant is collected, and the radioactivity is measured with a gamma counter (COBRAII AUTO-GAMMA, MODEL D5005, manufactured by Packard Instrument Company). The cytotoxic activity (%) can be calculated using the measured values according to the equation: (A−C)/(B−C)×100, wherein A represents the radioactivity (cpm) in each sample, B represents the radioactivity (cpm) in a sample where 1% NP-40 (manufactured by Nacalai Tesque) has been added, and C represents the radioactivity (cpm) of a sample containing the target cells only.

Meanwhile, in the case of CDC activity measurement, 50 μL of target cell and 50 μL of an anti-Claudin 3 antibody are added to a 96-well flat-bottomed plate (manufactured by Becton Dickinson), and reacted for 15 minutes on ice. Thereafter, 100 μL of the complement solution is added, and incubated in a carbon dioxide incubator for four hours. The final concentration of the antibody is adjusted to 0 or 3 μg/mL. After incubation, 100 μL of supernatant is collected, and the radioactivity is measured with a gamma counter. The cytotoxic activity can be calculated in the same way as in the ADCC activity determination.

On the other hand, in the case of measuring the cytotoxic activity of an antibody conjugate, 50 μL of target cell and 50 μL of an anti-Claudin 3 antibody conjugate are added to a 96-well flat-bottomed plate (manufactured by Becton Dickinson), and reacted for 15 minutes on ice. This is then incubated in a carbon dioxide incubator for one to four hours. The final concentration of the antibody is adjusted to 0 or 3 μg/mL. After culturing, 100 μL of supernatant is collected, and the radioactivity is measured with a gamma counter. The cytotoxic activity can be calculated in the same way as in the ADCC activity determination.

In the present invention, the cells whose proliferation is suppressed by a monoclonal antibody are not particularly limited, as long as the cells express a Claudin 3 protein. Preferred Claudin 3-expressing cells are, for example, cancer cells. More preferably, the cells are ovarian cancer, prostate cancer, breast cancer, uterine cancer, liver cancer, lung cancer, pancreatic cancer, stomach cancer, bladder cancer, and colon cancer cells. Therefore, anti-Claudin 3 antibodies can be used for the purpose of treating or preventing cell proliferation-induced diseases such as ovarian cancer, prostate cancer, breast cancer, uterine cancer, liver cancer, lung cancer, pancreatic cancer, stomach cancer, bladder cancer, and colon cancer.

### Pharmaceutical Compositions

In another aspect, the present invention provides pharmaceutical compositions comprising a monoclonal antibody that binds to a Claudin 3 protein as an active ingredient. Furthermore, the present invention relates to anticancer agents comprising a monoclonal antibody that binds to a Claudin 3 protein as an active ingredient. Cell proliferation inhibitors and anticancer agents of the present invention are preferably administered to subjects affected with cancer, or subjects with the likelihood of recurrence of cancer.

Furthermore, in the present invention, an anticancer agent comprising a monoclonal antibody that binds to a Claudin 3 protein as an active ingredient can also be described as a method for preventing or treating cancer which comprises the step of administering an antibody that binds to a Claudin 3 protein to a subject, or as use of a monoclonal antibody that binds to a Claudin 3 protein in the production of an anticancer agent.

In the present invention, the phrase “comprising a monoclonal antibody that binds to Claudin 3 as an active ingredient” means comprising an anti-Claudin 3 monoclonal antibody as the major active ingredient, and does not limit the content percentage of the monoclonal antibody.

Furthermore, multiple types of monoclonal antibodies can be mixed into the pharmaceutical compositions or anticancer agents of the present invention as necessary. For example, the cytotoxic effect against Claudin 3-expressing cells may be strengthened by producing a cocktail of multiple Claudin 3-binding monoclonal antibodies. Alternatively, the therapeutic effect can be enhanced by mixing a Claudin 3-binding antibody with an antibody that recognizes another tumor-related antigen.

The monoclonal antibody included in the pharmaceutical composition of the present invention (for example, cell proliferation inhibitor and anticancer agent; same hereinafter) is not particularly limited as long as it binds to a Claudin 3 protein, and examples include antibodies described herein.

The pharmaceutical compositions or anticancer agents of the present invention can be administered orally or parenterally to a patient. Preferably, the administration is parenteral administration. Specifically, the method of administration is, for example, administration by injection, transnasal administration, transpulmonary administration, or transdermal administration. Examples of administration by injection include systemic and local administrations of a pharmaceutical composition of the present invention by intravenous injection, intramuscular injection, intraperitoneal injection, subcutaneous injection, or such. A suitable administration method may be selected according to the age of the patient and symptoms. The dosage may be selected, for example, within the range of 0.0001 mg to 1000 mg per kg body weight in each administration. Alternatively, for example, the dosage for each patient may be selected within the range of 0.001 to 100,000 mg/body. However, the pharmaceutical composition of the present invention is not limited to these doses.

The pharmaceutical compositions of the present invention can be formulated according to conventional methods (for example, Remington's Pharmaceutical Science, latest edition, Mark Publishing Company, Easton, U.S.A), and may also contain pharmaceutically acceptable carriers and additives. Examples include, but are not limited to, surfactants, excipients, coloring agents, perfumes, preservatives, stabilizers, buffers, suspending agents, isotonization agents, binders, disintegrants, lubricants, fluidity promoting agents, and flavoring agents; and other commonly used carriers can be suitably used. Specific examples of the carriers include light anhydrous silicic acid, lactose, crystalline cellulose, mannitol, starch, carmellose calcium, carmellose sodium, hydroxypropyl cellulose, hydroxypropyl methyl cellulose, polyvinylacetal diethylaminoacetate, polyvinylpyrrolidone, gelatin, medium chain fatty acid triglyceride, polyoxyethylene hardened castor oil 60, saccharose, carboxymethyl cellulose, corn starch, inorganic salt, and such.

Furthermore, the present invention provides methods for inducing injury in Claudin 3-expressing cells or methods for suppressing cell proliferation by contacting the Claudin 3-expressing cells with monoclonal antibodies that bind to a Claudin 3 protein. The monoclonal antibodies that bind to a Claudin 3 protein are described above as Claudin 3-protein-binding antibodies contained in the cell proliferation inhibitors of the present invention. Cells to which the anti-Claudin 3 antibodies bind are not particularly limited, as long as the cells express Claudin 3. Preferred Claudin 3-expressing cells of the present invention are cancer cells. Specifically, ovarian cancer, prostate cancer, breast cancer, uterine cancer, liver cancer, lung cancer, pancreatic cancer, stomach cancer, bladder cancer, and colon cancer cells are suitable as Claudin 3-expressing cells of the present invention.

In the present invention “contacting” is accomplished, for example, by adding an antibody to a culture solution containing Claudin 3-expressing cells in a test tube. In this case, the antibody can be added in the form of, for example, a solution or a solid obtained by freeze-drying or the like. When adding the antibody as an aqueous solution, the aqueous solution used may purely contain only the antibody, or the solution may include, for example, the above-mentioned surfactants, excipients, coloring agents, perfumes, preservatives, stabilizers, buffers, suspending agents, isotonization agents, binders, disintegrants, lubricants, fluidity promoting agents, or flavoring agents. The concentration for addition is not particularly limited, but the final concentration in the culture that may be suitably used is preferably in the range of 1 pg/mL to 1 g/mL, more preferably 1 ng/mL to 1 mg/mL, and even more preferably 1 μg/mL to 1 mg/mL.

Furthermore, in another embodiment, “contacting” in the present invention is carried out by administration to a non-human animal to which a Claudin 3-expressing cell has been transplanted into the body, or to an animal carrying cancer cells endogenously expressing Claudin 3. The method of administration may be oral or parenteral administration. The method of administration is particularly preferably parenteral administration, and specifically, the method of administration is, for example, administration by injection, transnasal administration, transpulmonary administration, or transdermal administration. Examples of administration by injection include systemic and local administrations of pharmaceutical compositions, cell proliferation inhibitors and anticancer agents of the present invention by intravenous injection, intramuscular injection, intraperitoneal injection, subcutaneous injection, or such. A suitable administration method may be selected according to the age of the test animal and symptoms. When administering as an aqueous solution, the aqueous solution used may purely contain only the antibody, or the solution may include, for example, the above-mentioned surfactants, excipients, coloring agents, perfumes, preservatives, stabilizers, buffers, suspending agents, isotonization agents, binders, disintegrants, lubricants, fluidity promoting agents, or flavoring agents. The dosage may be selected, for example, within the range of 0.0001 mg to 1000 mg per kg body weight in each administration. Alternatively, for example, the dosage for each patient may be selected within the range of 0.001 to 100,000 mg/body. However, the antibody dose of the present invention is not limited to these doses.

The following method is suitably used as a method for evaluating or measuring cell damage induced by contacting Claudin 3-expressing cells with an anti-Claudin 3 antibody. Examples of a method for evaluating or measuring the cytotoxic activity in a test tube include methods for measuring the above-mentioned antibody-dependent cell-mediated cytotoxicity (ADCC) activity, complement-dependent cytotoxicity (CDC) activity, and such. Whether or not an anti-Claudin 3 antibody has ADCC activity or CDC activity can be measured by known methods (for example, Current protocols in Immunology, Chapter 7. Immunologic studies in humans, Editor, John E. Coligan et al., John Wiley & Sons, Inc., (1993) and the like). For activity measurements, an binding antibody having the same isotype as anti-Claudin 3 antibody but not having any cytotoxic activity can be used as a control antibody in the same manner as the anti-Claudin 3 antibody, and it can be determined that the activity is present when the anti-Claudin 3 antibody shows a stronger cytotoxic activity than the control antibody.

The isotype of an antibody is defined by the sequence of its H chain constant region in the antibody amino acid sequence. The isotype of an antibody is ultimately determined in vivo by class switching that arises from genetic recombinations in chromosomes which occur during maturation of antibody-producing B-cells. Difference in isotype is reflected in the difference of physiological and pathological functions of antibodies. Specifically, for example, the strength of cytotoxic activity is known to be influenced by antibody isotype in addition to the expression level of the antigen. Therefore, when measuring the above-described cell damaging activity, an antibody of the same isotype as the test antibody is preferably used as the control.

To evaluate or measure cell damaging activity in vivo, for example, Claudin 3-expressing cancer cells are intradermally or subcutaneously transplanted to a non-human test animal, and then a test antibody is intravenously or intraperitoneally administered daily or at the interval of few days, starting from the day of transplantation or the following day. Cytotoxicity can be defined by daily measurement of tumor size. In a similar manner to the evaluation in a test tube, cytotoxicity can be determined by administering a control antibody having the same isotype, and observing that the tumor size in the anti-Claudin 3 antibody-administered group is significantly smaller than the tumor size in the control antibody-administered group. When using a mouse as the non-human test animal, it is suitable to use a nude (nu/nu) mouse whose thymus has been made genetically defective so that its T lymphocyte function is lost. The use of such a mouse can eliminate the participation of T lymphocytes in the test animals when evaluating or measuring the cytotoxicity of the administered antibody.

As a method for evaluating or measuring the suppressive effect on proliferation of Claudin 3-expressing cells by contact with an anti-Claudin 3 antibody, a method of measuring the uptake of isotope-labeled thymidine into cells or the MTT method may be suitably used.

Furthermore, as a method for evaluating or measuring the cell proliferation-suppressing activity in vivo, the above-described method for evaluating or measuring the cytotoxic activity in vivo can be suitably used.

All prior art references cited herein are incorporated by reference into this description.

### EXAMPLES

Herein below, the present invention will be specifically described with reference to the Examples, but it is not to be construed as being limited thereto.

### Example 1

### Gene Cloning and Production of Forced Expression Cells

The genes encoding human Claudin 3, Claudin 1, Claudin 4, Claudin 6, and mouse Claudin 3 were cloned, their mammalian cell expression vectors were constructed, and their forced expression cells were established. PCR primers were designed based on the respective GenBank reference sequences, and PCR amplification of the respective genes was performed using gene-expressing tissue cDNA libraries as template, and the genes of interest were isolated. Sequences of the obtained genes were confirmed by DNA sequencing analysis. The primers and cDNA libraries (Marathon cDNA libraries from Clonetech) used for the gene cloning are shown in Table 1.

TABLE 1

| | | | GenBank | cDNA library | | |
|---|---|---|---|---|---|---|
| | Gene name | | reference | for gene | Cloning | Cloning |
| Species | (SEQ ID NO:) | Abbreviation | sequence ID | cloning | primer 1 | primer 2 |
| Human | claudin 3 | hCLDN3 | NM_001303 | Human kidney | 5′-CGGCCACCATGTCCATG | 5′-GTCTGTCCCTTAGACGT |
| | (SEQ ID NO: 1) | | | | GGCCTGGAGATCA-3′ | AGTCCTTGCGGTC-3′ |
| | | | | | (SEQ ID NO: 119) | (SEQ ID NO: 120) |
| Human | claudin 1 | hCLDN1 | NM_021101 | Human liver | 5′-ATGGCCAACGCGGGGCT | 5′-TGTGTCACACGTAGTCT |
| | (SEQ ID NO: 5) | | | | GCAGCTGTTGGGC-3′ | TTCCCGCTGGAAG-3′ |
| | | | | | (SEQ ID NO: 123) | (SEQ ID NO: 124) |
| Human | claudin 4 | hCLDN4 | NM_001305 | Human | 5′-GAACAATGGCCTCCATG | 5′-AGGAGGGTGGACTCTG |
| | (SEQ ID NO: 7) | | | fetal lung | GGGCTACAGG-3′ | TTCTTGCTAGCAG-3′ |
| | | | | | (SEQ ID NO: 125) | (SEQ ID NO: 126) |
| Human | claudin 6 | hCLDN6 | NM_021195 | Human | 5′-CATGGCCTCTGCCGGAA | 5′-CCCAAAGCTGTTGGGC |
| | (SEQ ID NO: 9) | | | fetal lung | TGCAGATCCT-3′ | ACTGCCACTTC-3′ |
| | | | | | (SEQ ID NO: 127) | (SEQ ID NO: 128) |
| Mouse | claudin 3 | mCLDN3 | NM_009902 | Mouse liver | 5′-GCGAATTCCACCATGTC | 5′-GCGATATCTGTCCTCT |
| | (SEQ ID NO: 3) | | | | CATGGGCCTGGAGATCA-3′ | TCCAGCCTAGCAAGCAG-3′ |
| | | | | | (SEQ ID NO: 121) | (SEQ ID NO: 122) |

The obtained genes were inserted into a mammalian expression vector in which a gene was transcribed under the mouse CMV promoter. The nucleotide sequences of the inserted cDNAs used for the construction of expression vectors are shown in SEQ ID NOs: 1, 3, 5, 7, and 9. All of these expression vectors, except for the vectors for human and mouse Claudin 3, have a nucleotide sequence encoding a FLAG tag attached to the C terminus of the recombinant proteins. The expression vectors were introduced into the cell lines listed below by the electroporation method. Ba/F3 is a mouse lymphocyte-derived cancer cell line. The other, DG44 is a Chinese hamster ovary (CHO) cell-derived dihydrofolate reductase-deficient (dhfr⁻) cell line. The respective transformed cells were selected on Geneticin (Invitrogen) resistance, which is conferred by the expression vectors.

Ba/F3 (RIKEN Biosource Center, Cell No. RCB0805)

### DG44 (Invitrogen, 12613014)

The presence or absence of recombinant protein expression in Geneticin-resistant cell clones was judged by the SDS-PAGE/Western blotting method. The human Claudin 1, Claudin 4, and Claudin 6 proteins were detected by an anti-FLAG M2 antibody (Sigma), of which the C terminal FLAG expression tags were added by genetic engineering. The protein expression of human and mouse Claudin 3 was detected with an anti-Claudin 3 antibody (Zymed, 34-1700). Clones that showed highest expression level of each protein were selected, and the cell lines were cultured, maintained, and used for subsequent experiments.

### Example 2

### Establishment of Anti-Claudin 3 Monoclonal Antibody Hybridomas

Mice were immunized by the DNA immunization method using the Helios gene gun system (Bio-Rad) to establish hybridomas producing anti-Claudin 3 monoclonal antibodies.

The expression vectors used for DNA immunization were constructed as follows. A cDNA of human Claudin 3 was amplified by PCR from a kidney cDNA library (Clontech). The PCR was conducted by using an LA Taq DNA polymerase reaction solution (Takara) with the cDNA amplification primers, cloning primer 1 and cloning primer 2 (shown in Table 1). The amplified cDNA fragments were cloned into the pGEM-T Easy vector, and the nucleotide sequences were determined. A cDNA fragment containing Claudin 3 was excised using EcoRI, and this fragment was inserted into the EcoRI site of pMC, which is a mammalian expression vector, to obtain an expression vector (full-length human Claudin 3 expression vector) for DNA immunization. The nucleotide sequence of the full-length human Claudin 3 expression vector is shown in SEQ ID NO: 159. In the nucleotide sequence of SEQ ID NO: 159, the nucleotide sequence of positions 836 to 1498 encodes the amino acid sequence of Claudin 3.

The cartridge tubing was coated with gold-DNA (full length human Claudin 3 expression vector) particles according to the Helios gene gun operation manual. 50 mg of 1.0-μm gold particles were weighed out, and suspended by mixing in 0.1 mL of 0.05 M spermidine solution. 0.1 mL of 1 mg/mL plasmid solution was added to this, and then vortexed. Subsequently, 0.1 mL of 1 M CaCl₂ was added, and this was left to stand for ten minutes. After brief centrifugation, the supernatant was removed, and the pellet was suspended in ethanol, and then this was centrifuged. After repeating the ethanol dehydration step three times, this was ultimately suspended in 6 mL of 0.05 mg/mL polyvinylpyrollidone/ethanol solution. This solution was drawn into the tubing for coating, and the tubing was coated, dried, and cut into 0.5-inch-long segments using a tube cutter.

DNA immunization was performed on four- to five-weeks-old mice (Charles River Japan, MRL/MpJ-Tnfrsf6^{1pr}/Cr1j) (approximately 200 psi helium pressure) for one to three times per week, and the anti-Claudin 3 antibody titer in the serum was monitored intermittently during this period. Cells forced to express Claudin 3 (5×10⁶ cells/head) were administered intraperitoneally to individuals confirmed to have an increased serum antibody titer. After rearing for two to three days, the spleen was extirpated, and mononuclear cells containing antibody-producing cells were isolated. Spleen-derived cells were mixed with P3-X63Ag8U.1 (ATCC CRL-1597) at an approximately 2:1 ratio, and cell fusion was carried out by gradual addition of PEG 1500 (Roche Diagnostics). RPMI1640 medium (GIBCO BRL) was added carefully to dilute PEG 1500, and then PEG 1500 was removed by centrifugation. Then, the cells were seeded into a 96-well culture plate at 200 μL/well in RPMI1640 medium containing the following components (hereinafter referred to as HAT medium), and cultured at 37° C. under 5% CO₂ for approximately one week:

### 10% FBS;

1×HAT media supplement (SIGMA); and
0.5×BM-Condimed H1 Hybridoma cloning supplement (Roche Diagnostics).

After confirming the formation of hybridoma colonies under a microscope, the presence or absence of Claudin 3-binding antibodies in the culture supernatant was screened by a flow cytometry method using cells forced to express Claudin 3. The mouse antibodies bound to the forced expression cells were measured by FACSCalibur (Becton Dickinson) using an FITC-labeled goat anti-mouse IgG antibody (Beckman Coulter) as secondary antibodies. The selective binding of the antibodies to Claudin 3 was judged by comparison of the forced expression cells and the non-recombinant parental cells, and the hybridomas from positive wells were cloned by the limiting dilution method.

Not many hybridomas producing Claudin 3-binding antibodies could be obtained from mice immunized by intraperitoneal administration of cells forced to express Claudin 3. In contrast, hybridomas producing Claudin 3-binding antibodies could be efficiently obtained from mice subjected to DNA immunization followed by intraperitoneal administration of cells forced to express Claudin 3.

The antibody isotype of the hybridoma clones was determined using the Mouse Monoclonal Antibody Isotyping Kit (Roche Diagnostics).

For purification of monoclonal antibodies, hybridomas were cultured in a HAT medium supplemented with Ultra low IgG FBS (Invitrogen), and the culture supernatants were harvested. Antibodies belonging to the IgG1, IgG2a, and IgG2b subtypes were purified using HiTrap Protein G HP (Amersham Biosciences) according to the manufacturer's instructions. Antibodies belonging to the IgG3 and IgM subtypes were purified using a Protein L Agarose (Sigma) column under conditions similar to those for Protein G. The solvent of the elution fractions was replaced with PBS using a PD-10 column (Amersham Bioscience). Then, the purified antibodies were concentrated by ultrafiltration, and stored at 4° C. The antibody concentration was determined by the Bradford method using mouse IgG as the standard.

### Example 3

### Analysis of the Binding Specificity of the Monoclonal Antibodies

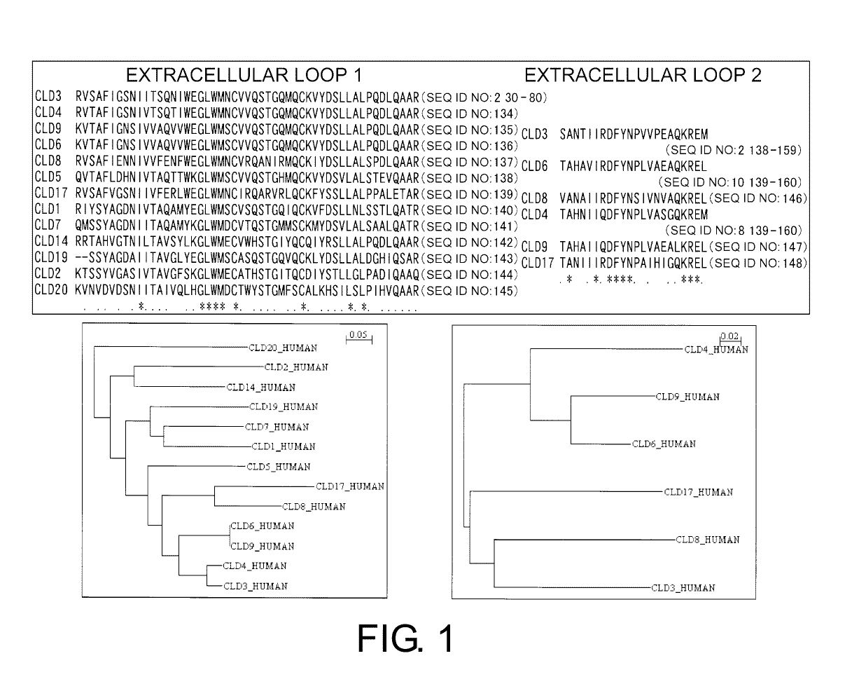

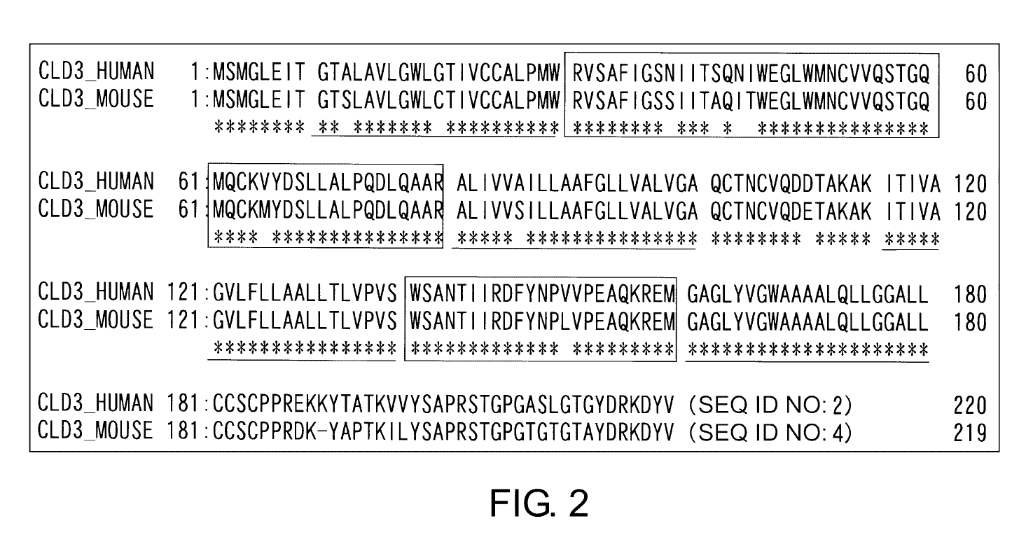

24 Claudin family genes are present on the human chromosome. Claudin 4 is highly homologous to Claudin 3 in the full-length amino acid sequence. To characterize the redundancy and specificity of cell surface epitopes recognized by monoclonal antibodies, a sequence alignment and clustering diagrams of the putative extracellular sequences of Claudin 3 and the corresponding sequences of highly homologous family molecules were depicted (FIG. 1). The family molecule showing the highest identity in the extracellular loop 1 is Claudin 4 (48 residues out of 51 residues are identical). In the second place, Claudin 6 and Claudin 9 have a high identity to Claudin 3 (41 residues out of 51 residues are identical). Compared to the extracellular loop 1 region, the sequences in the extracellular loop 2 region is not conserved (15 residues out of 22 residues are identical between Claudin 6 and Claudin 8). Conservation between the sequences of human and mouse Claudin 3 is high; 46 out of 51 residues are identical in the extracellular loop 1 region, and 22 out of 23 residues are identical in the extracellular loop 2 region (FIG. 2).

To verify the specificity of monoclonal antibodies and categorize the epitopes, the binding reactivity to cells forced to express Claudin 3 and cells forced to express the following highly homologous Claudin family molecules was evaluated by the flow cytometry method:

mouse Claudin 3;

human Claudin 1;

human Claudin 4; and

human Claudin 6.

A monoclonal antibody was added to the respective forced expression cells, and then incubated at 4° C. for 30 minutes. After incubation, the cells were washed once with a PBS solution containing 1% fetal bovine serum, then a 150-fold dilution of an FITC-goat anti-mouse IgG (H+L) antibody (Beckman Coulter) was added, and this was incubated at 4° C. for 30 minutes. The amount of antibodies bound per cell was measured using FACSCalibur, and the X geometric mean value, which is the geometric mean of the cell fluorescence intensity, was calculated using the accessory CellQuest Pro analysis software of FACSCalibur. The results are summarized in Table 2.

TABLE 2

| | | hCLN3/DG44 | | | MCF7 | | hCLN3 | mCLN3 | hCLN1 | hCLN4 | hCLN6 | Ba/F3 |
|---|---|---|---|---|---|---|---|---|---|---|---|---|
| | Isotype | 5 μg/ml | 1 μg/ml | 0.1 μg/ml | 5 μg/ml | 1 μg/ml | 2 μg/ml | | | | | |
| CDN01 | IgG2b/IgK | 456 | 176 | 27 | 24 | 10 | 197 | 23 | 9 | 10 | 8 | 10 |
| CDN02 | IgG2b/IgK | 1757 | 531 | 66 | 313 | 143 | 1464 | 15 | 14 | 137 | 8 | 10 |
| CDN03 | IgM/IgK | 724 | 542 | 94 | 35 | 29 | 474 | 348 | 10 | 17 | 9 | 11 |
| CDN04 | IgG1/IgK | 667 | 180 | 28 | 130 | 47 | 366 | 16 | 11 | 14 | 9 | 8 |
| CDN05 | IgM/IgK | 169 | 70 | 18 | 71 | 46 | 59 | 119 | 9 | 148 | 11 | 10 |
| CDN07 | IgG1/IgK | 351 | 109 | 26 | 79 | 34 | 238 | 76 | 12 | 14 | 9 | 10 |
| CDN08 | IgG2b/IgK | 2014 | 809 | 105 | 321 | 125 | 1380 | 12 | 14 | 47 | 8 | 10 |
| CDN16 | IgG2b/IgK | 2482 | 928 | 125 | 361 | 125 | 1975 | 1270 | 13 | 11 | 9 | 10 |
| CDN17 | IgM/IgK | 866 | 567 | 88 | 299 | 173 | 1028 | 342 | 9 | 156 | 10 | 11 |
| CDN24 | IgM/IgK | 1129 | 694 | 101 | 382 | 286 | 1114 | 502 | 18 | 293 | 13 | 12 |
| CDN27 | IgG2a/IgK | 2696 | 1793 | 308 | 310 | 202 | 3344 | 50 | 42 | 302 | 25 | 48 |
| CDN28 | IgG1/IgK | 1990 | 1452 | 275 | 186 | 175 | 1427 | 82 | 12 | 27 | 9 | 11 |
| CDN29 | IgG1/IgK | 1643 | 851 | 126 | 87 | 60 | 1689 | 65 | 11 | 12 | 10 | 11 |
| CDN30 | IgG2a/IgK | 1663 | 615 | 93 | 58 | 29 | 1718 | 176 | 27 | 38 | 19 | 30 |
| CDN31 | IgG2a/IgK | 1917 | 649 | 86 | 231 | 101 | 1598 | 31 | 35 | 174 | 22 | 43 |
| CDN32 | IgG1/IgK | 951 | 354 | 63 | 80 | 49 | 818 | 27 | 11 | 11 | 10 | 10 |
| CDN33 | IgG3/IgK | 547 | 406 | 65 | 77 | 45 | 576 | 17 | 11 | 12 | 9 | 11 |
| CDN35 | IgG2a/IgK | 2297 | 869 | 120 | 380 | 176 | 2147 | 1310 | 69 | 59 | 36 | 44 |
| CDN36 | IgG1/IgK | 986 | 384 | 68 | 46 | 18 | 909 | 60 | 11 | 11 | 11 | 11 |
| CDN37 | IgG2a/IgK | 1134 | 544 | 90 | 52 | 21 | 388 | 44 | 26 | 66 | 22 | 31 |
| CDN38 | IgG3/IgK | 258 | 215 | 207 | 61 | 63 | 142 | 115 | 7 | 74 | 9 | 8 |
| Control | | 12 | | | 9 | | 28 | 12 | 10 | 9 | 8 | 13 |

When the antibodies were added to DG44 forced to express human Claudin 3 at a final concentration of 5, 1, or 0.1 μg/mL, the amounts of bound antibodies increased in a dose dependent manner for all of the antibodies. The cross reactivity for human Claudin 3, human Claudin 1, human Claudin 4, human Claudin 6, and mouse Claudin 3 forcedly expressed in Ba/F3 was evaluated using at a final antibody concentration of 2 μg/mL. All of the isolated antibodies bound more strongly to cells forced to express human Claudin 3, as compared to the Ba/F3 cells which is a parental cell line accommodating the forced expression. On the other hand, all of the antibodies hardly bound to cells forced to express human Claudin 1 or human Claudin 6, which have lower sequence identity to Claudin 3. The antibodies that showed high and selective affinity to Claudin 3 are listed below:

CDN08, CDN16, CDN14, CDN28, CDN29,

CDN30, CDN32, CDN33, and CDN36.

CDN16 and CDN35 showed nearly equivalent high affinity to human and mouse Claudin 3. Specific antibodies recognizing a common epitope among animal species may be useful as tools for studying the difference between efficacy and toxicity in pathologic model animals. That is, when an antibody that specifically recognizes an epitope whose sequence is conserved among animal species is administered to pathologic model animals, the pharmacokinetics of the antibody is expected to show similar behavior in the animal species for which the disease is treated. CDN02, CDN05, CDN17, CDN24, CDN27, and CDN31, which bind to human Claudin 3, were also shown to bind to human Claudin 4, and this suggests that the antibodies recognize a sequence structure that is common or very similar between the two proteins.

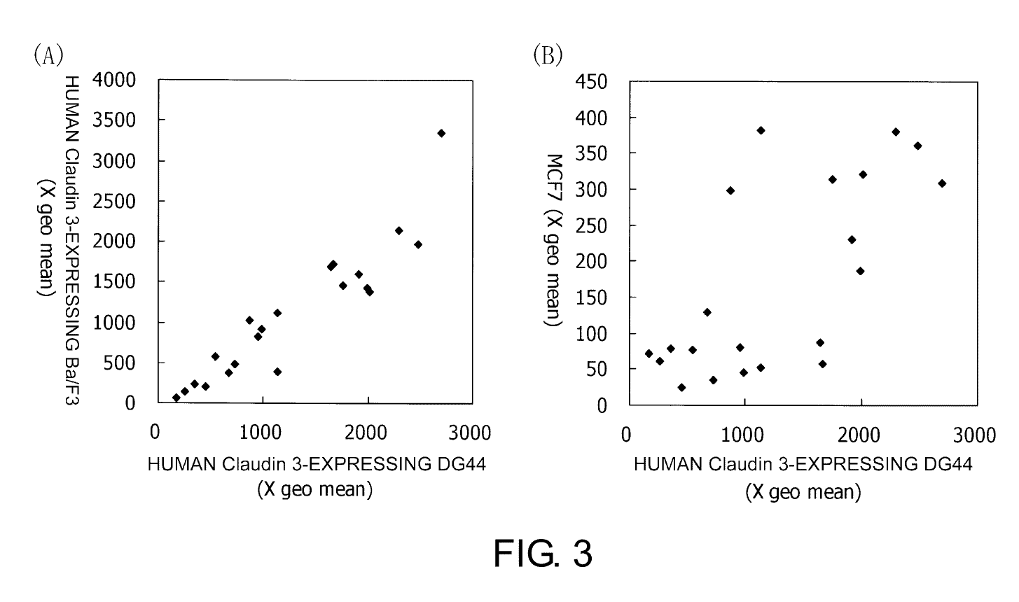

The affinity of the antibodies to the MCF7 breast cancer cell line (ATCC, HTB-22), which endogenously expresses Claudin 3, was evaluated by the flow cytometry method. While the antibody concentration-dependent elevation of the binding level was observed as in the forced expression cells, the preference of binding to MCF7 did not necessarily correlate with the result for the forced expression cells (FIG. 3). This suggests that the manner in which the epitopes are exposed could be different between the forced expression cells and the cancer cell line.

### Example 4

### Affinity of the Monoclonal Antibodies to the Extracellular Loop Region Sequence Peptides

In general, it is said that even when an antibody can be successfully obtained by immunization with a short loop of a multi-transmembrane protein in the form of a linear peptide, such an antibody scarcely binds to the naturally-occurring protein with high affinity. On the other hand, an antibody that binds to a rigid portion in a tertiary structure may hardly bind to a linearized peptide. The isolated monoclonal antibodies bind to Claudin 3 expressed on cells, and their affinity for linarized peptides that correspond to the extracellular loop sequences was evaluated using GST/extracellular loop peptide fusion proteins. The portions predicted to be the extracellular regions of human Claudin 3 are shown below.

Loop 1: the sequence of amino acid residue numbers 30 to 80 in SEQ ID NO: 2

Loop 2: the sequence of amino acid residue numbers 137 to 159 in SEQ ID NO: 2

In the pGEX-4T2 Escherichia coli expression vector, an expression unit was engineered so that the GST protein is fused to the N terminus of the loop sequences, and a His tag is attached to the C terminus of the loop sequences. The protein expression was induced in E. coli, and the fusion proteins were purified. The amino acid sequences of the loop 1 and loop 2 fusion proteins are shown in SEQ ID NO: 116 and SEQ ID NO: 117, respectively. The loop 1 fusion protein was accumulated in E. coli as an insoluble protein. Thus, after disrupting the E. coli, the insoluble fraction was collected, solubilized in 7 M urea, and then the protein was purified using a nickel affinity column in the presence of urea. After elution by imidazole, urea was removed by dialysis against 50 mM Tris-HCl (pH 8). Since the loop 2 fusion protein was expressed in a soluble fraction, the fusion protein was purified by glutathione affinity chromatography. Nunc-Immuno plates were coated with the purified fusion proteins. After blocking with a solution containing BSA, the binding reactivity of the monoclonal antibodies was evaluated. An anti-His mouse monoclonal antibody (Santa Cruz) was used as a positive control antibody that binds to the fusion proteins.

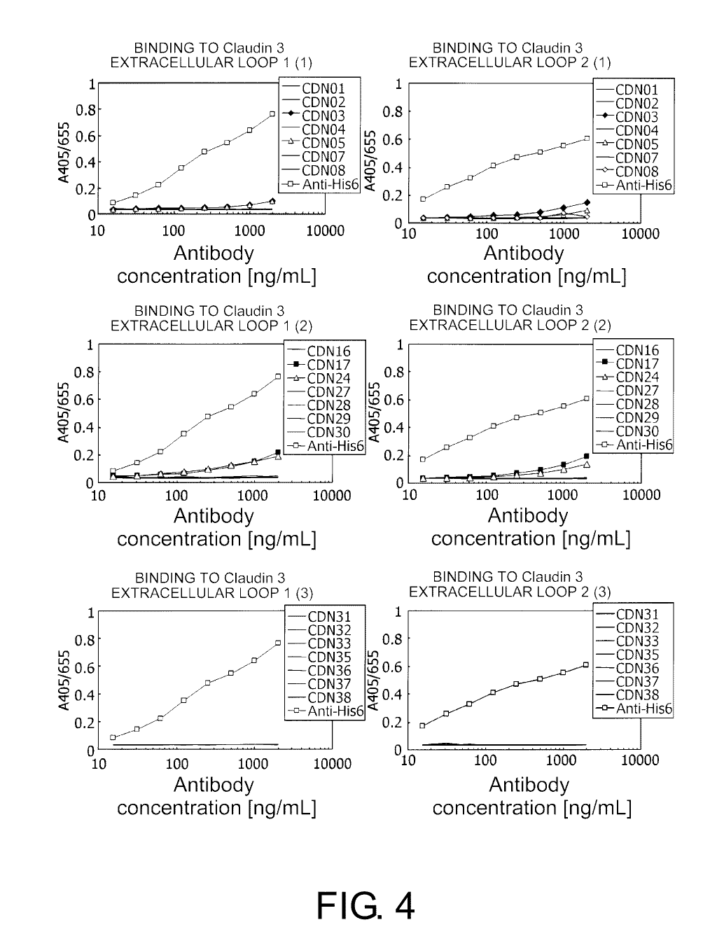

After one hour of incubation, the plates were washed, and an alkaline phosphatase-labeled anti-mouse IgG (H+L) antibody was added and reacted. After washing, the amount of antibody bound was measured by adding the Sigma 104 detection reagent (FIG. 4). While the positive control anti-His antibody bound to the fusion proteins on the plates, the anti-Claudin 3 monoclonal antibodies hardly bound to the loop 1 or loop 2 peptide fragment. Some antibodies showed weak binding to the loop 1 and loop 2 fusion proteins, they bound to both the proteins with equivalent affinity, and no binding specificity was observed. The above-mentioned results suggest that all of the antibodies isolated in the present invention bind to a rigid portion of the tertiary structure of the Claudin 3 protein.

Considering that the only previously reported Claudin 3-binding polyclonal antibody was obtained by peptide immunization and peptide affinity purification, it is clear that the mode of binding to the antigen of the reported antibody differs from that of the antibodies isolated in the present invention. It was confirmed that the monoclonal antibodies of the present invention are useful in that the antibodies bind more efficiently to Claudin 3 expressed on cells.

### Example 5

### Induction of Cytotoxicity by the Monoclonal Antibodies

The selective complement-dependent cytotoxicity activity of the monoclonal antibodies in Claudin 3-expressing cells was evaluated using a baby rabbit complement. DG44 cells forced to express human Claudin 3 were used as human Claudin 3-expressing cells, and the parental DG44 cells were used as the control. After addition of a purified monoclonal antibody at a final reaction concentration of 5 μg/mL, the cells were incubated at 4° C. for 30 minutes. Then, Baby Rabbit Complement (Cederlane, Cat. No. CL3441) was added at a final concentration of 1%, and this was incubated at 37° C. under 5% CO₂ for 90 minutes.

After incubation, 7-aminoactinomycin D (7-AAD, Invitrogen), which is a DNA-binding fluorescent reagent, was added at a final concentration of 1 μg/mL, and this was left to stand in the dark for ten minutes. After centrifugation, the supernatant was removed, and the cells were suspended in PBS containing 1% fetal bovine serum, and the fluorescence intensity of the cells stained was measured using a flow cytometer. The instrument and gating measurement conditions were set in advance, so that the percentage of positive cells stained with 7-AAD will be 5% or less under the conditions without addition of an antibody or a complement. The complement-dependent cytotoxicity activity of the antibodies was measured.

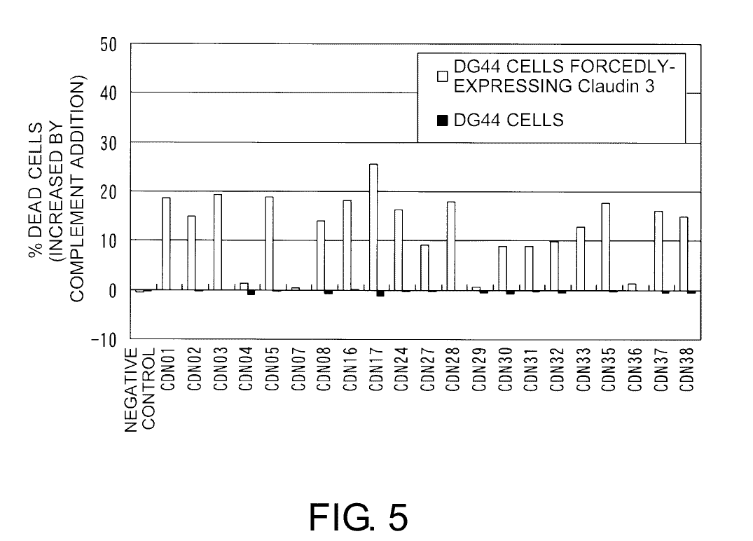

As in the case without antibody addition, cell injury was hardly induced in the parental DG44 cells by the antibodies. In contrast, all of the antibodies, except for the antibodies of the IgG1 subtype, showed cytotoxic activity against DG44 cells forced to express human Claudin 3 (FIG. 5). Although the antibody subtype of CDN28 and CDN32 is IgG1, these antibodies induced cytotoxicity. As described above, many of the antibodies isolated in the present invention were shown to induce antigen expression-dependent and complement-dependent cytotoxicity.

The complement-dependent cytotoxicity activity of the monoclonal antibodies against the MCF7 breast cancer cell line was evaluated by the chromium release method. RPMI1640 medium (Invitrogen) containing 10% fetal bovine serum and 10 μg/mL human insulin was used to maintain MCF7. MCF7 cells were seeded onto a 96-well plate, and cultured overnight. Then, Chromium-51 (Code No. CJS4, Amersham Biosciences) was added, and the cells were incubated for a few more hours. After washing the cells with the medium, fresh medium was added. Then, the anti-Claudin 3 monoclonal antibodies and the control mouse IgG2a antibody were added to the wells. The final concentration of the antibodies was adjusted to 10 μg/mL. Subsequently, a baby rabbit complement was added at a final concentration of 2%, and then the plate was left to stand in a 5% carbon dioxide gas incubator at 37° C. for 1.5 hours. Thereafter, the plate was centrifuged (1000 rpm for five minutes at 4° C.), 100 μL of the supernatant was collected from each well, and its radioactivity was measured using a gamma counter (1480 WIZARD 3″, Wallac). The specific chromium release rate was determined based on the following equation:

Specific chromium release rate (%)=(A−C)×100/(B−C)

where A, B, and C show values for the following:
A—the radioactivity (cpm) in each well;
B—the mean value of radioactivity (cpm) in wells where 100 μL of 2% NP-40 solution (Nonidet P-40, Code No. 252-23, Nacalai Tesque) was added to 100 μL of cells; and
C—the mean value of radioactivity (cpm) in wells where 100 μL of the medium was added to 100 μL of cells.

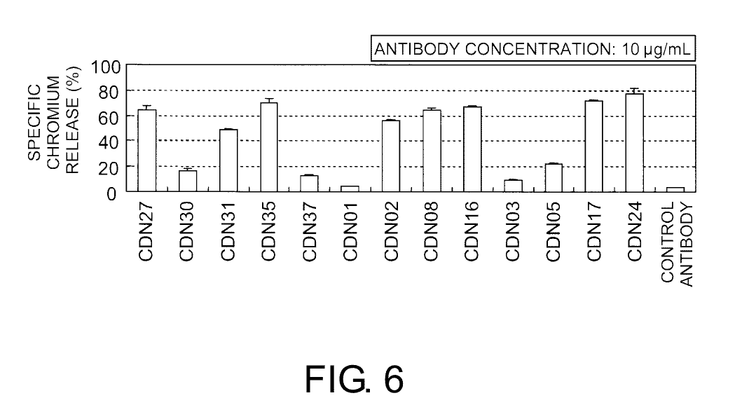

The measurements were conducted in triplicate for each experimental condition, and the mean value and standard deviation were calculated for the specific chromium release rate (FIG. 6). The following monoclonal antibodies showed strong complement-dependent cytotoxicity activity against MCF7:

CDN27, CDN31, CDN35, CDN02,

CDN08, CDN16, CDN17, and CDN24.

The strength of cytotoxicity activity closely correlated with the amount of bound antibody as measured by the flow cytometry method. On the other hand, the control mouse IgG2a antibody did not show complement-dependent cytotoxicity activity at the same concentration.

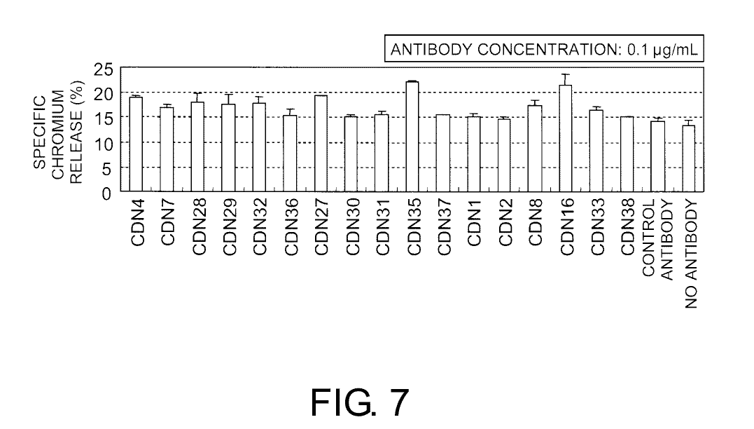

Using MCF7 cells as the target, the antibody-dependent cytotoxicity activity was measured by the chromium release method. Cells were cultured in a 96-well flat-bottomed plate. After reaction with Chromium-51, the cells were washed with RPMI1640 medium, and 100 μL of fresh medium was added. Then, the anti-Claudin 3 monoclonal antibodies and the control (no antibody) were added at a final concentration of 0.1 μg/mL. Subsequently, a solution containing effector cells, the number of which is approximately 50-times that of MCF7, was added to each well, and the plate was incubated at 37° C. in a 5% carbon dioxide gas incubator. For the effector cells, spleen cells of C3H/HeNCrlCrlj mice (Charles River Japan) cultured in a medium containing 50 ng/mL of recombinant interleukin-2 (Cat. No. 200-02, PeproTech) were used. After letting the plate stand for six hours, the specific chromium release rate was measured, and the mean and standard deviation were calculated (FIG. 7).

Compared to no addition of antibody and addition of the control antibody (IgG2a subtype), addition of CDN04, CDN27, CDN35, and CDN16 induced chromium release. Thus, these antibodies were confirmed to have antibody-dependent cell-mediated cytotoxicity activity against Claudin 3-expressing cells.

### Example 6

### Cloning of Antibody Variable Regions and Production of Recombinant Antibodies

cDNAs encoding the antibody variable regions were cloned using the SMART RACE cDNA Amplification kit (Clonetech), and the nucleotide sequences were determined. Total RNAs were purified using RNeasy Mini (Qiagen) from cultured hybridoma cells. From this RNA, cDNAs were synthesized according to the SMART RACE cDNA Amplification Kit manual, and the cDNAs of the antibody gene variable regions were amplified by PCR using subtype-specific primers. The subtype-specific primer sequences used for the amplification are shown in Table 3.

TABLE 3

| | |
|---|---|
| Antibody subtype | Primer sequence |

| | | |
|---|---|---|
| IgG1 | 5′-CCATGGAGTTAGTTTGGGCAGCAGATCC-3′ | (SEQ ID NO: 129) |
| IgG2a | 5′-CAGGGGCCAGTGGATAGACCGATG-3′ | (SEQ ID NO: 130) |
| IgG2b | 5′-CAGGGGCCAGTGGATAGACTGATG-3′ | (SEQ ID NO: 131) |
| IgG3 | 5′-ATGTGTCACTGCAGCCAGGGACCAA-3′ | (SEQ ID NO: 188) |
| IgK | 5′-GGCACCTCCAGATGTTAACTGCTCACT-3′ | (SEQ ID NO: 132) |
| IgL | 5′-TCGAGCTCTTCAGAGGAAGGTGGAAAC-3′ | (SEQ ID NO: 133) |

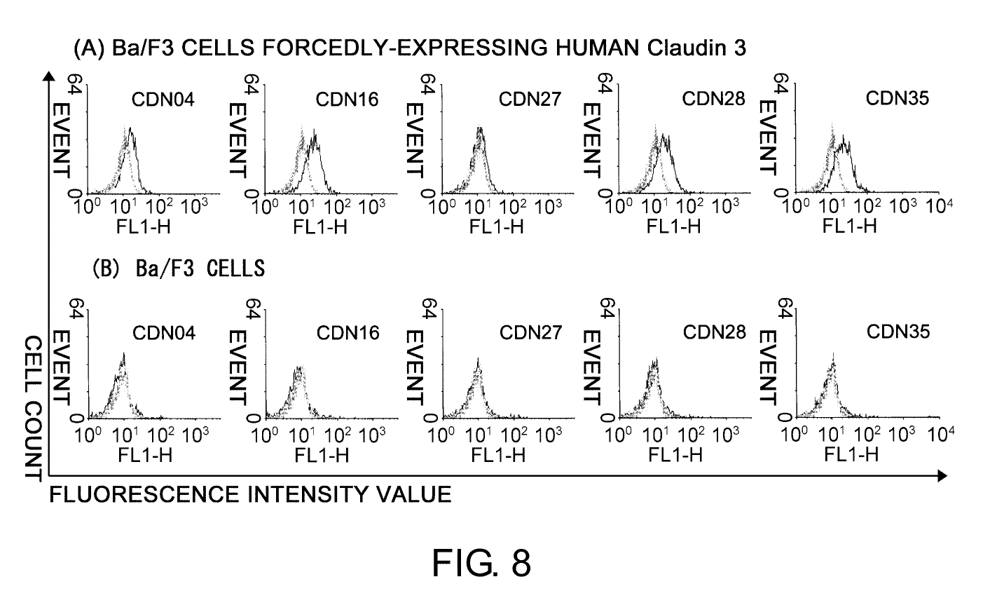

The fragments were amplified using Takara Ex Taq DNA polymerase (Takara), and cloned into the pGEM-T Easy vector, and the nucleotide sequences were determined. Recombinant antibody expression vectors were constructed from the isolated antibody variable region sequences. In brief, individually-cloned heavy-chain and light-chain variable region sequences were linked in translational frame with the human antibody IgG1 constant region and human IgK constant region sequences, respectively. In expression vectors constructed, the mouse-human chimeric antibody genes are transcribed under the mouse CMV promoter. Cells transiently-expressing recombinant antibodies were obtained by introducing the expression vectors into COS7 cells. Flow cytometric data using the supernatants obtained after two days of culturing and anti-human IgG (H+L)-FITC as the secondary antibody demonstrated that the recombinant antibodies bind specifically to cells forced to express Claudin 3 (FIG. 8).

### Example 7

### Analysis of the Binding of the Monoclonal Antibodies to the Loops Displayed on Cells

As described above, the monoclonal antibodies of the present invention do not show affinity to the GST fusion protein, comprising linearized peptides that are putative extracellular loop. To obtain information on the epitopes of these monoclonal antibodies, each antibody was analyzed to determine whether it binds to loop 1 or loop 2. The monoclonal antibodies isolated in the present invention hardly bound to human Claudin 1. A chimeric molecule carrying loop 1 of Claudin 3 and loop 2 of Claudin 1 (CLD1/3), and a chimeric molecule carrying loop 1 of Claudin 1 and loop 2 of Claudin 3 (CLD3/1) were expressed in cells, and the affinity of the antibodies to the cells was evaluated by flow cytometry. If an antibody binds to CLD1/3-expressing cells but not to CLD3/1-expressing cells, this means that the antibody binds to loop 2. If an antibody binds to CLD3/1-expressing cells but not to CLD1/3-expressing cells, this means that the antibody binds to loop 1.

Comparison of the amino acid sequences of Claudin 3 and Claudin 1 shows that a common sequence motif “FLLA” is present in the third putative transmembrane region. This portion was used as the boundary to design chimeric constructs, in which the amino acid sequences before and after the boundary derived from different proteins are linked together. Information on the amino acid sequences of the designed chimeric molecules is set forth below:

CLD1/3 protein: positions 1-127 of the Claudin 1 amino acid sequence and positions 126-220 of the Claudin 3 amino acid sequence

CLD3/1 protein: positions 1-125 of the Claudin 3 amino acid sequence and positions 128-211 of the Claudin 1 amino acid sequence

The nucleotide sequence and amino acid sequence of each chimeric molecule are shown in the following sequence ID numbers:

| | Nucleotide sequence | Amino acid sequence |
|---|---|---|
| CLD 1/3 protein: | SEQ ID NO: 160 | SEQ ID NO: 161 |
| CLD 3/1 protein: | SEQ ID NO: 162 | SEQ ID NO: 163 |

In brief, the genes were constructed as follows. Using the Claudin 1 and 3 cDNA sequences as templates, partial gene fragments were amplified by PCR, and the gene fragments were linked by PCR assembly to produce a gene of chimeric molecule. The genes were inserted in translational frame into a mammalian cell expression vector designed for addition of a FLAG tag to the C terminus. The vector was introduced into Ba/F3 cells to obtain drug-resistant cell clones. Protein expression in the drug-resistant clone was confirmed by Western blotting using an anti-FLAG antibody, and chimeric molecule-expressing cells were established by selecting a clone with high expression level.

Epitope analysis of the monoclonal antibodies using the chimeric molecule-expressing cells showed unexpected results. Many of the monoclonal antibodies bound strongly to Claudin 3 (CLD3/3) having the naturally-occurring amino acid sequence. Specifically, in the results of FACS shown in FIG. 9, clear peaks with fluorescence signal were detected. On the other hand, most of the monoclonal antibodies did not bind at all to both the chimeric protein-expressing cells used in the experiment. Peaks with low fluorescence were observed for some of the antibodies, indicating only weak binding to the cells. For example, weak binding was observed between CDN16 and CLD1/3, and between CDN35 and CLD1/3. A similar tendency was also observed for anti-Claudin 3 mouse antiserum. It was presumed that the above results were not due to a screening bias at the establishment of hybridoma, but that both loop 1 and loop 2 are necessary for an antibody to strongly bind to Claudin 3 expressed on cells.

### Example 8

### Production of Cell Lines Stably Expressing an Anti-Claudin 3 Chimeric Antibody

DG44 cells were transformed with a human chimeric antibody expression vector by the electroporation method. Recombinant cell clones were selected based on the geneticin resistance acquired by a selection marker present on the human chimeric antibody expression vector. The antibodies in the culture supernatant of the recombinant clones was quantified by sandwich ELISA using anti-human antibodies, and recombinant antibody-expressing cells were selected. Human chimeric antibodies were purified from the culture supernatant of the selected recombinant cells using a HiTrap Protein A column (Amersham Bioscience) according to the attached manual.

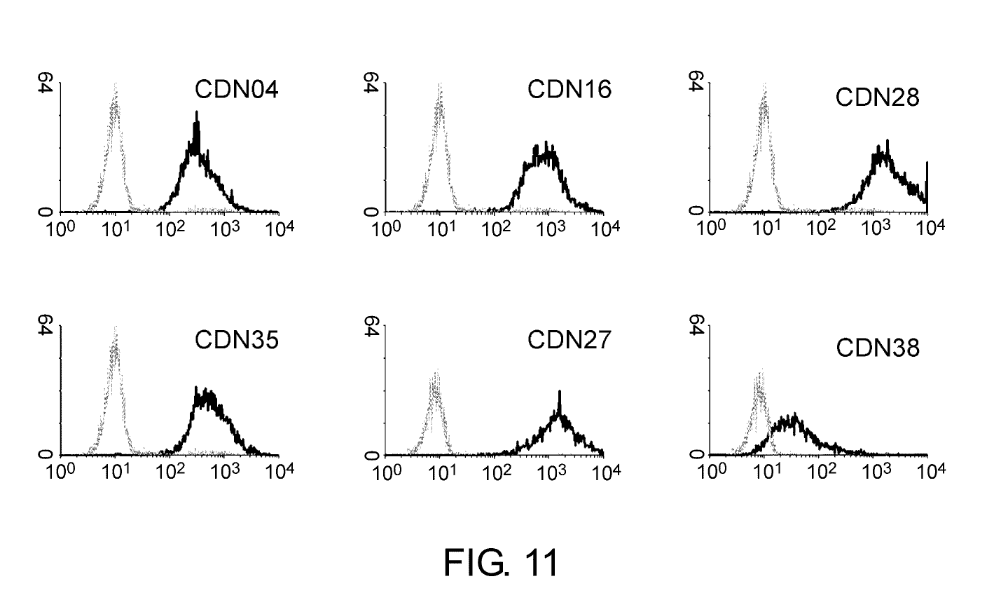

The affinity of the human chimeric antibodies to DG44 cells forced to express Claudin 3 and Ba/F3 cells forced to express Claudin 3 was evaluated by flow cytometry. The chimeric antibodies were added to and reacted with the cells forced to express Claudin 3, and then the bound chimeric antibodies were detected using anti-human IgG (H+L)-FITC. As shown in FIG. 11, remarkable shifts by chimeric antibody addition were observed in the histograms, and thus the chimeric antibodies were confirmed to bind to Claudin 3.

### Example 9

### Inhibition of Colony Formation in Soft Agar and Cell Migration by Addition of Anti-Claudin 3 Antibodies

Agarwal and others reported that overexpression of Claudin 3 and Claudin 4 is involved in the enhancement of survival capacity and acquisition of invasion ability of ovarian cancer cells, from analyses using forced expression of Claudin 3 and 4 and small interfering RNAs against Claudin 3 and 4 (Agarwal et al. (2005) Cancer Res 65, 7378-7385). On the other hand, Michl and others reported that overexpression of Claudin 4 suppresses the metastatic and infiltration ability of pancreatic cancer cells (Michl et al. (2003) Cancer Res 63, 6265-6271).

The effect of the presence or absence of the expression, or increase or decrease in the expression was evaluated in the above-mentioned reports. No report has shown that cell functions can be modified by an antibody that binds to a Claudin 3 protein. To see whether the survival capacity or invasion ability of cancer cells can be altered by binding of anti-Claudin 3 antibodies, the effect of antibody addition on the ability of MCF7 cells to form colonies in soft agar and to migrate was assessed.

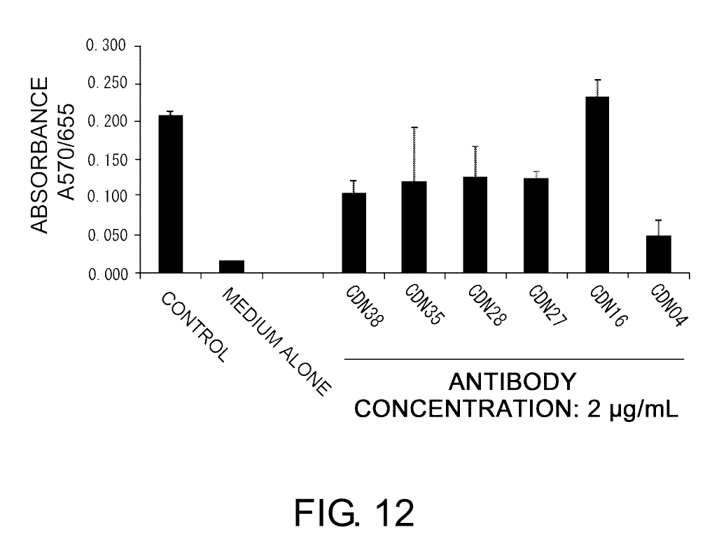

The effect of antibody addition on colony formation in soft agar was evaluated using CytoSelect 96-well In Vitro Tumor Sensitivity Assay (Cell Biolabs, Inc.). 5000 MCF7 cells per well were seeded into soft agar together with a mouse antibody, and cultured for seven days at 37° C. under 5% CO₂. After culturing, the number of cells was quantified by the MTT method (FIG. 12). Colony formation was suppressed by addition of anti-Claudin 3 antibodies, and this effect was particularly strong with CDN04.

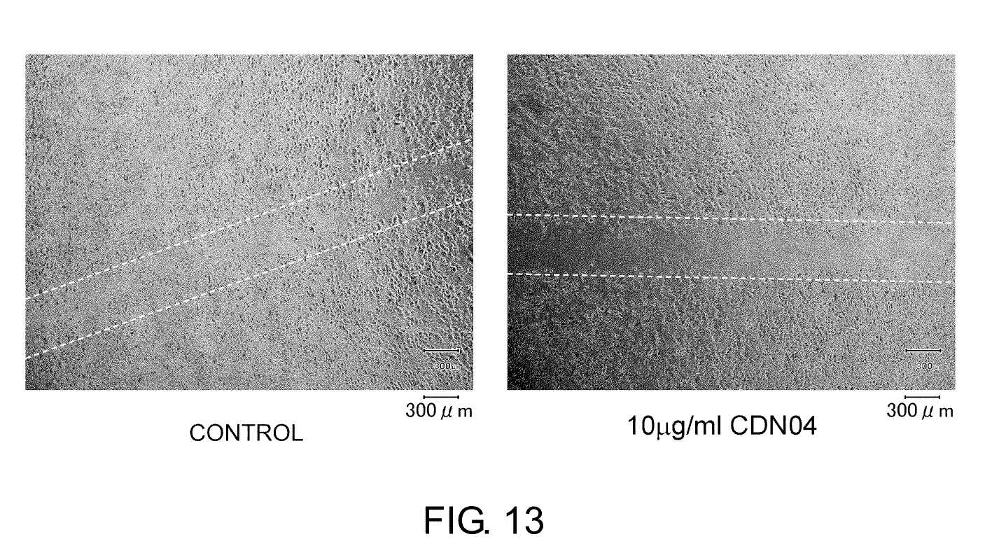

The effect of antibody addition on cell motility was evaluated by the following method (wound-healing assay). MCF7 cells were seeded into a 12-well plastic plate, and culturing was continued until the density of cells capable of attached growth became saturated. The cell monolayer was linearly scratched with the edge of a pipette tip. After replacement of medium, the antibodies were added at a final concentration of 10 μg/mL, and the cells were continuously cultured for four days. After incubation, cells migrated to cover the wounded region in the control wells without antibody addition. On the other hand, in the wells to which the CDN04 antibody (10 μg/mL) was added, cell migration to the wounded region was hardly observed (FIG. 13). No significant inhibition of cell migration was observed in the wells to which CDN16, CDN27, CDN28, CDN35, or CDN38 was added.

This example demonstrates for the first time that Claudin 3-binding antibodies can regulate cellular functions such as anchorage-independent proliferation and cell migration, which are characteristics of cancer cells.

### INDUSTRIAL APPLICABILITY

The present invention provides anti-Claudin 3 monoclonal antibodies. Since Claudin 3 shows high sequence identity among species, it was not easy to obtain such antibodies by conventional immunization methods. Therefore, it is highly significant that the present invention provides Claudin 3-recognizing antibodies. In particular, in a preferred embodiment, the monoclonal antibodies provided by the present invention can bind to Claudin 3 expressed on the cell surface, but no substantial reactivity to linear peptides comprising amino acid sequences of the extracellular domains of Claudin 3 was observed. That is, the monoclonal antibodies of the present invention are antibodies that cannot be obtained by domain peptide immunization using the amino acid sequences of the extracellular domains.

Many of the molecules belonging to the Claudin family are structurally similar. In addition, since the lengths of the extracellular domains are short, it was expected to be difficult to obtain antibodies that can distinguish individual Claudin family molecules expressed on cell surface. However, in a preferred embodiment, the monoclonal antibodies provided by the present invention can immunologically distinguish between Claudin 3 and Claudin 6. Among the 51 residues of the amino acid sequence constituting extracellular loop 1 of Claudin 3, 41 residues are shared with the amino acid sequence of extracellular loop 1 of Claudin 6. It can be said that antibodies that can immunologically distinguish molecules sharing high identity as such are antibodies with excellent specificity.

Therefore, the present invention provides antibodies that can specifically recognize and bind to Claudin 3 expressed on the surface of cancer cells. Antibodies of the present invention can detect cancers that overexpress Claudin 3. For example, the expression of Claudin 3 has been shown to be elevated in ovarian cancer, prostate cancer, breast cancer, uterine cancer, liver cancer, lung cancer, pancreatic cancer, stomach cancer, bladder cancer, colon cancer, and such. Thus, monoclonal antibodies of the present invention are useful for diagnosis of cancers that have enhanced expression of Claudin 3, such as ovarian cancer, prostate cancer, breast cancer, uterine cancer, liver cancer, lung cancer, pancreatic cancer, stomach cancer, bladder cancer, and colon cancer.

Furthermore, in a preferred embodiment, the monoclonal antibodies of the present invention were confirmed to show cytotoxic action against Claudin 3-expressing cells. Specifically, for example, monoclonal antibodies having CDC activity and ADCC activity against breast cancer are provided. Furthermore, in a preferred embodiment, the monoclonal antibodies of the present invention recognize not only Claudin 3 but also Claudin 4. The expression of the Claudin 4 gene was reported to be elevated at chemotherapeutic agent-resistant recurrent sites in uterine cancer patients.

Therefore, the monoclonal antibodies of the present invention were shown to be useful for treatment of cancers that overexpress Claudin 3 or Claudin 4. Furthermore, the monoclonal antibodies of the present invention retain the activity to bind to Claudin 3 even after chimerization by substituting the constant-region sequences with human-derived amino acid sequences. This confirms that the monoclonal antibodies provided by the present invention can be chimerized and made into cancer therapeutic antibodies that can be administered to humans. More specifically, monoclonal antibodies of the present invention are useful for treatment of cancers that have enhanced expression of either one or both of Claudin 3 and Claudin 4, such as ovarian cancer, prostate cancer, breast cancer, uterine cancer, liver cancer, lung cancer, pancreatic cancer, stomach cancer, bladder cancer, and colon cancer.

1. A monoclonal antibody that binds to a Claudin 3 protein.

2. The monoclonal antibody of claim 1, wherein the antibody binds to a protein expressed on the cell membrane and comprising the amino acid sequence of SEQ ID NO: 2.

3. The monoclonal antibody of claim 2, wherein the antibody does not substantially cross-react with a peptide comprising the amino acid sequence of positions 30 to 80 or positions 137 to 159 in the amino acid sequence of SEQ ID NO: 2.

4. The monoclonal antibody of claim 1, wherein the antibody binds to a protein expressed on the cell membrane and comprising the amino acid sequence of SEQ ID NO: 4.

5. The monoclonal antibody of claim 1, wherein the antibody binds to a protein expressed on the cell membrane and comprising the amino acid sequence of SEQ ID NO: 8.

6. An antibody that binds to a protein expressed on the cell membrane and comprising the amino acid sequence of any one of SEQ ID NOs: 2, 4, and 8, by recognizing a conformation formed by two extracellular loops of the protein.

7. The antibody of claim 6, wherein the antibody does not substantially cross-react with a peptide comprising the amino acid sequence of positions 30 to 80 or positions 137 to 159 in the amino acid sequence of SEQ ID NO: 2.

8. The antibody of claim 6, wherein the antibody is a monoclonal antibody.

9. The antibody of claim 1 or 6, wherein the antibody has cytotoxic activity.

10. The antibody of claim 9, wherein the cytotoxic activity is ADCC activity.

11. The antibody of claim 9, wherein the cytotoxic activity is CDC activity.

12. The antibody of claim 1 or 6, wherein a chemotherapeutic agent or a toxic peptide is bound to the antibody.

13. An antibody that binds to a Claudin 3 protein, wherein a cytotoxic substance selected from the group consisting of a chemotherapeutic agent, toxic peptide, and radioisotope is bound to the antibody.

14. The antibody of claim 1, 6, or 13, which is an antibody described in any of (1) to (61) below:
(1) an antibody comprising an H chain having the amino acid sequence of SEQ ID NO: 12 as CDR1, the amino acid sequence of SEQ ID NO: 14 as CDR2, and the amino acid sequence of SEQ ID NO: 16 as CDR3;
(2) an antibody comprising the H chain of (1), wherein the H chain has the amino acid sequence of positions 139 to 462 in the amino acid sequence of SEQ ID NO: 20 as CH;
(3) an antibody comprising the H chain of (1), wherein the H chain has the amino acid sequence of SEQ ID NO: 22 as CH;
(4) an antibody comprising an L chain having the amino acid sequence of SEQ ID NO: 24 as CDR1, the amino acid sequence of SEQ ID NO: 26 as CDR2, and the amino acid sequence of SEQ ID NO: 28 as CDR3;
(5) an antibody comprising the L chain of (4), wherein the L chain has the amino acid sequence of positions 135 to 240 in the amino acid sequence of SEQ ID NO: 32 as CL;
(6) an antibody comprising the L chain of (4), wherein the L chain has the amino acid sequence of SEQ ID NO: 34 as CL;
(7) an antibody comprising the H chain of (1) and the L chain of (4);
(8) an antibody comprising the H chain of (2) and the L chain of (5);
(9) an antibody comprising the H chain of (3) and the L chain of (6);
(10) an antibody having one or more amino acid substitutions, deletions, additions, and/or insertions in the antibody of any of (1) to (9), which has equivalent activity as the antibody of any of (1) to (9);
(11) an antibody comprising an H chain having the amino acid sequence of SEQ ID NO: 36 as CDR1, the amino acid sequence of SEQ ID NO: 38 as CDR2, and the amino acid sequence of SEQ ID NO: 40 as CDR3;
(12) an antibody comprising the H chain of (11), wherein the H chain has the amino acid sequence of positions 140 to 476 in the amino acid sequence of SEQ ID NO: 44 as CH;
(13) an antibody comprising the H chain of (11), wherein the H chain has the amino acid sequence of SEQ ID NO: 22 as CH;
(14) an antibody comprising an L chain having the amino acid sequence of SEQ ID NO: 46 as CDR1, the amino acid sequence of SEQ ID NO: 48 as CDR2, and the amino acid sequence of SEQ ID NO: 50 as CDR3;
(15) an antibody comprising the L chain of (14), wherein the L chain has the amino acid sequence of positions 133 to 238 in the amino acid sequence of SEQ ID NO: 54 as CL;
(16) an antibody comprising the L chain of (14), wherein the L chain has the amino acid sequence of SEQ ID NO: 34 as CL;
(17) an antibody comprising the H chain of (11) and the L chain of (14);
(18) an antibody comprising the H chain of (12) and the L chain of (15);
(19) an antibody comprising the H chain of (13) and the L chain of (16);
(20) an antibody having one or more amino acid substitutions, deletions, additions, and/or insertions in the antibody of any of (11) to (19), which has equivalent activity as the antibody of any of (11) to (19);
(21) an antibody comprising an H chain having the amino acid sequence of SEQ ID NO: 56 as CDR1, the amino acid sequence of SEQ ID NO: 58 as CDR2, and the amino acid sequence of SEQ ID NO: 60 as CDR3;
(22) an antibody comprising the H chain of (21), wherein the H chain has the amino acid sequence of positions 137 to 471 in the amino acid sequence of SEQ ID NO: 64 as CH;
(23) an antibody comprising the H chain of (21), wherein the H chain has the amino acid sequence of SEQ ID NO: 22 as CH;
(24) an antibody comprising an L chain having the amino acid sequence of SEQ ID NO: 66 as CDR1, the amino acid sequence of SEQ ID NO: 68 as CDR2, and the amino acid sequence of SEQ ID NO: 70 as CDR3;
(25) an antibody comprising the L chain of (24), wherein the L chain has the amino acid sequence of positions 133 to 238 in the amino acid sequence of SEQ ID NO: 74 as CL;
(26) an antibody comprising the L chain of (24), wherein the L chain has the amino acid sequence of SEQ ID NO: 34 as CL;
(27) an antibody comprising the H chain of (21) and the L chain of (24);
(28) an antibody comprising the H chain of (22) and the L chain of (25);
(29) an antibody comprising the H chain of (23) and the L chain of (26);
(30) an antibody having one or more amino acid substitutions, deletions, additions, and/or insertions in the antibody of any of (21) to (29), which has equivalent activity as the antibody of any of (21) to (29);
(31) an antibody comprising an H chain having the amino acid sequence of SEQ ID NO: 76 as CDR1, the amino acid sequence of SEQ ID NO: 78 as CDR2, and the amino acid sequence of SEQ ID NO: 80 as CDR3;
(32) an antibody comprising the H chain of (31), wherein the H chain has the amino acid sequence of positions 140 to 463 in the amino acid sequence of SEQ ID NO: 84 as CH;
(33) an antibody comprising the H chain of (31), wherein the H chain has the amino acid sequence of SEQ ID NO: 22 as CH;
(34) an antibody comprising an L chain having the amino acid sequence of SEQ ID NO: 86 as CDR1, the amino acid sequence of SEQ ID NO: 88 as CDR2, and the amino acid sequence of SEQ ID NO: 90 as CDR3;
(35) an antibody comprising the L chain of (34), wherein the L chain has the amino acid sequence of positions 133 to 238 in the amino acid sequence of SEQ ID NO: 94 as CL;
(36) an antibody comprising the L chain of (34), wherein the L chain has the amino acid sequence of SEQ ID NO: 34 as CL;
(37) an antibody comprising the H chain of (31) and the L chain of (34);
(38) an antibody comprising the H chain of (32) and the L chain of (35);
(39) an antibody comprising the H chain of (33) and the L chain of (36);
(40) an antibody having one or more amino acid substitutions, deletions, additions, and/or insertions in the antibody of any of (31) to (39), which has equivalent activity as the antibody of any of (31) to (39);
(41) an antibody comprising an H chain having the amino acid sequence of SEQ ID NO: 96 as CDR1, the amino acid sequence of SEQ ID NO: 98 as CDR2, and the amino acid sequence of SEQ ID NO: 100 as CDR3;
(42) an antibody comprising the H chain of (41), wherein the H chain has the amino acid sequence of positions 140 to 474 in the amino acid sequence of SEQ ID NO: 104 as CH;
(43) an antibody comprising the H chain of (41), wherein the H chain has the amino acid sequence of SEQ ID NO: 22 as CH;
(44) an antibody comprising an L chain having the amino acid sequence of SEQ ID NO: 106 as CDR1, the amino acid sequence of SEQ ID NO: 108 as CDR2, and the amino acid sequence of SEQ ID NO: 110 as CDR3;
(45) an antibody comprising the L chain of (44), wherein the L chain has the amino acid sequence of positions 133 to 238 in the amino acid sequence of SEQ ID NO: 114 as CL;
(46) an antibody comprising the L chain of (44), wherein the L chain has the amino acid sequence of SEQ ID NO: 34 as CL;
(47) an antibody comprising the H chain of (41) and the L chain of (44);
(48) an antibody comprising the H chain of (42) and the L chain of (45);
(49) an antibody comprising the H chain of (43) and the L chain of (46);
(50) an antibody having one or more amino acid substitutions, deletions, additions, and/or insertions in the antibody of any of (41) to (49), which has equivalent activity as the antibody of any of (41) to (49);
(51) an antibody comprising an H chain having the amino acid sequence of SEQ ID NO: 167 as CDR1, the amino acid sequence of SEQ ID NO: 169 as CDR2, and the amino acid sequence of SEQ ID NO: 171 as CDR3;
(52) an antibody comprising the H chain of (51), wherein the H chain has the amino acid sequence of positions 118 to 447 in the amino acid sequence of SEQ ID NO: 173 as CH;
(53) an antibody comprising the H chain of (51), wherein the H chain has the amino acid sequence of SEQ ID NO: 22 as CH;
(54) an antibody comprising an L chain having the amino acid sequence of SEQ ID NO: 179 as CDR1, the amino acid sequence of SEQ ID NO: 181 as CDR2, and the amino acid sequence of SEQ ID NO: 183 as CDR3;
(55) an antibody comprising the L chain of (54), wherein the L chain has the amino acid sequence of positions 113 to 218 in the amino acid sequence of SEQ ID NO: 185 as CL;
(56) an antibody comprising the L chain of (54), wherein the L chain has the amino acid sequence of SEQ ID NO: 34 as CL;
(57) an antibody comprising the H chain of (51) and the L chain of (54);
(58) an antibody comprising the H chain of (52) and the L chain of (55);
(59) an antibody comprising the H chain of (53) and the L chain of (56);
(60) an antibody having one or more amino acid substitutions, deletions, additions, and/or insertions in the antibody of any of (51) to (59), which has equivalent activity as the antibody of any of (51) to (59);
(61) an antibody that binds to the same epitope as the Claudin 3 protein epitope bound by the antibody of any of (1) to (60).

15. A method for diagnosis of cancer, comprising the step of binding the antibody of claim 1 to a Claudin 3 protein.

16. A method for diagnosis of cancer, comprising the steps of:
(a) collecting a sample from a subject; and
(b) detecting a Claudin 3 protein contained in the collected sample using an antibody that binds to the Claudin 3 protein.

17. A method for diagnosis of cancer, comprising the steps of: (1) administering to a subject a radioisotope-labeled antibody that binds to a Claudin 3 protein; and (2) detecting accumulation of said radioisotope.

18. The diagnostic method of claim 17, wherein the radioisotope is a positron-emitting nuclide.

19. The diagnostic method of claim 18, wherein the positron-emitting nuclide is any nuclide selected from the group consisting of ¹¹C, ¹³N, ¹⁵O, ¹⁸F, ⁴⁵Ti, ⁵⁵Co, ⁶⁴Cu, ⁶⁶Ga, ⁶⁸Ga, ⁷⁶Br, ⁸⁹Zr, and ¹²⁴I.

20. The diagnostic method of claim 15 or 16, wherein the cancer is any cancer selected from the group consisting of ovarian cancer, prostate cancer, breast cancer, uterine cancer, liver cancer, lung cancer, pancreatic cancer, stomach cancer, bladder cancer, and colon cancer.

21. The diagnostic method of claim 20, wherein the cancer is primary cancer.

22. The diagnostic method of claim 20, wherein the cancer is metastatic cancer.

23. A diagnostic agent for use in a method of cancer diagnosis, which comprises an antibody that binds to a Claudin 3 protein.

24. A kit for use in a method of cancer diagnosis, which comprises an antibody that binds to a Claudin 3 protein, and a biological sample comprising a Claudin 3 protein.

25. A pharmaceutical composition comprising an antibody that binds to a Claudin 3 protein as an active ingredient.

26. A cell proliferation inhibitor comprising an antibody that binds to a Claudin 3 protein as an active ingredient.

27. An anticancer agent comprising an antibody that binds to a Claudin 3 protein as an active ingredient.

28. The anticancer agent of claim 27, wherein the cancer is any cancer selected from the group consisting of ovarian cancer, prostate cancer, breast cancer, uterine cancer, liver cancer, lung cancer, pancreatic cancer, stomach cancer, bladder cancer, and colon cancer.

29. The anticancer agent of claim 28, wherein the cancer is primary cancer.

30. The anticancer agent of claim 28, wherein the cancer is metastatic cancer.

31. The anticancer agent of claim 28, wherein said antibody is an antibody that binds to a protein expressed on the cell membrane and comprising the amino acid sequence of any one of SEQ ID NOs: 2, 4, and 8.

32.-33. (canceled)

34. A method of suppressing cell proliferation, which comprises the step of administering to a subject an antibody that binds to a Claudin 3 protein.

35. A method of preventing or treating cancer, which comprises the step of administering to a subject an antibody that binds to a Claudin 3 protein.

36. A method of inducing cell injury in a cell expressing a Claudin 3 protein, which comprises the step of contacting a Claudin 3 protein-expressing cell with an antibody that binds to a Claudin 3 protein.

37. A method of suppressing the proliferation of a cell expressing a Claudin 3 protein, which comprises the step of contacting a Claudin 3 protein-expressing cell with an antibody that binds to a Claudin 3 protein.
